(19) United States
(12) Patent Application Publication
(10) Pub. No.: US 2003/0139460 A1
(43) Pub. Date: Jul. 24, 2003

(54) 9-oxa-epothilon derivatives, method for the production and use thereof in pharmaceutical preparations

(76) Inventors: Wolfgang Schwede, Berlin (DE); Ulrich Klar, Berlin (DE); Werner Skuballa, Berlin (DE); Bernd Buchmann, Hohen Neuendorf (DE); Jens Hoffmann, Muehlenbeck (DE); Rosemarie Lichtner, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/258,017

(22) PCT Filed: Apr. 19, 2001
(86) PCT No.: PCT/EP01/04551

(30) Foreign Application Priority Data
Apr. 20, 2000 (DE) 10020899.1

(51) Int. Cl. C07D 417/02; C07D 413/02; A61K 31/427; A61K 31/422; A61K 31/365

(52) U.S. Cl. 514/365; 514/450; 514/374; 548/181; 548/236; 549/268

(57) ABSTRACT

The invention relates to novel epothilon derivatives which are characterized by an oxygen atom in position 9 of the epothilon skeleton. The novel compounds interact with tubulin, stabilizing formed microtubuli. They can influence cell division in a phase-specific manner and are suitable for use in the treatment of malignant tumors such as ovarian, stomach, colon, adeno, breast, lungs, head and neck carcinoma, malignant melanoma, acute lymphocytic and myelocytic leukaemia. They are also suitable for anti-angiogenesis thereapy and for use in the treatment of chronic inflamatory diseases (psoriasis, arthritis). In order to avoid uncontrolled proliferation of cells on and to improve the compatibility of medical implants, they can be placed on or in polymer materials. The inventive compounds can be used on their own or to obtain additive or synergistic effects in combination with other classes of substances and principles which can be used in tumoral therapy.

Höfle et al. describe the cytotoxic action of the natural products epothilone A (R=hydrogen) and epothilone B (R=methyl)

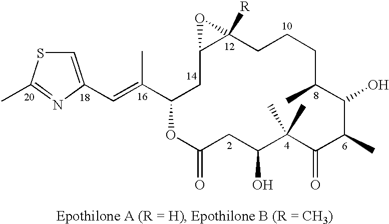

in, e.g., Angew. Chem. [Applied Chem.] 1996, 108, 1671-1673. Because of their in-vitro selectivity for breast cell lines and intestinal cell lines and their significantly higher activity against P-glycoprotein-forming multiresistant tumor lines in comparison to taxol as well as their physical properties that are superior to those of taxol, e.g., a water solubility that is higher by a factor of 30, this novel structural class is especially advantageous for the development of a pharmaceutical agent for treating malignant tumors.

The natural products are not sufficiently stable either chemically or metabolically for the development of pharmaceutical agents. To eliminate these drawbacks, modifications to the natural product are necessary. Such modifications are possible only with a total-synthesis approach and require synthesis strategies that make possible a broad modification of the natural product. The purpose of the structural changes is also to increase the therapeutic range. This can be done by improving the selectivity of the action and/or increasing the active strength and/or reducing undesirable toxic side effects, as they are described in Proc. Natl. Acad. Sci. USA 1998, 95, 9642-9647.

The total synthesis of epothilone A is described by Schinzer et al. in Chem. Eur. J. 1996, 2, No. 11, 1477-1482 and in Angew. Chem. 1997, 109, No. 5, pp. 543-544).

Epothilone derivatives were already described by Höfle et al. in WO 97/19086. These derivatives were produced starting from natural epothilone A or B. Also, epothilone C and D (double bond between carbon atoms 12 and 13: epothilone C=deoxyepothilone A; Epothilone D=deoxyepothilone B) are described as possible starting products in this respect.

Another synthesis of epothilone and epothilone derivatives was described by Nicolaou et al. in Angew. Chem. 1997, 109, No. 1/2, pp. 170-172. The synthesis of epothilones A and B and several epothilone analogs was described in Nature, Vol. 387, 1997, pp. 268-272; and the synthesis of epothilone A and its derivatives was described in J. Am. Chem. Soc., Vol. 119, No. 34, 1997, pp. 7960-7973 as well as the synthesis of epothilones A and B and several epothilone analogs in J. Am. Chem. Soc., Vol. 119, No. 34, 1997, pp. 7974-7991 also by Nicolaou et al.

Nicolaou et al. also describe in Angew. Chem. 1997, 109, No. 19, pp. 2181-2187 the production of epothilone A analogs using combinatory solid-phase synthesis. Several epothilone B analogs are also described there.

Epothilone derivatives, in some cases also epothilones C and D, are described in addition in Patent Applications WO 99/07692, WO 99/02514, WO 99/01124, WO 99/67252, WO 98/25929, WO 97/19086, WO 98/38192, WO 99/22461 and WO 99/58534.

The object of this invention consists in making available new epothilone derivatives, which are both chemically and metabolically stable enough for the development of pharmaceutical agents and which are superior to natural derivatives in terms of their therapeutic range, their selectivity of action and/or undesirable toxic side effects and/or their active strength.

This invention describes the new epothilone derivatives of general formula I,

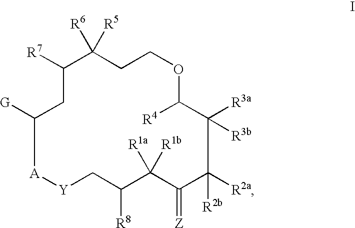

in which

R^{1a}, R^{1b} are the same or different and mean hydrogen, C₁-C₁₀-alkyl, aryl, C₇-C₂₀-aralkyl, or together a —(CH₂)ₘ group with m=1, 2, 3, 4 or 5, or a —CH₂—O—CH₂ group,

R^{2a}, R^{2b} are the same or different and mean hydrogen, C₁-C₁₀-alkyl, aryl, C₇-C₂₀-aralkyl, —(CH₂)ᵣ—C≡C—(CH₂)ₚ—R⁹, —(CH₂)ᵣ—CH═CH—(CH₂)ₚ—R⁹,

r is equal to 0 to 4,

p is equal to 0 to 3,

R⁹ is hydrogen, C₁-C₁₀-alkyl, aryl, C₇-C₂₀-aralkyl, C₁-C₁₀-acyl, or if p>0, also a group OR¹⁰,

R¹⁰ means hydrogen, or a protective group PG¹⁰,

R^{3a} means hydrogen, C₁-C₁₀-alkyl, aryl, C₇-C₂₀-aralkyl,

R^{3b} means OH, OPG³

R⁴ means hydrogen, C₁-C₁₀-alkyl, aryl, C₇-C₂₀-aralkyl,

R⁵ means hydrogen, C₁-C₁₀-alkyl, aryl, C₇-C₂₀-aralkyl, halogen, cyano, (CH₂)ₛ-T,

whereby s stands for 1, 2, 3, or 4,

T stands for OR¹¹ or Hal,

R¹¹ stands for hydrogen or PG¹¹,

R⁶, R⁷ each mean a hydrogen atom, together an additional bond or an oxygen atom,

G means a group

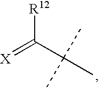

a bicyclic or tricyclic aryl radical,

R¹² means hydrogen, halogen, CN, C₁-C₂₀-alkyl, aryl, C₇-C₂₀-aralkyl, which can all be substituted,

X means an oxygen atom, two alkoxy groups OR¹³, a C₂-C₁₀-alkylene-α,ω-dioxy group, which can be straight-chain or branched, H/OR¹⁴ or a grouping CR¹⁵R¹⁶,

whereby

R¹³ stands for a C₁-C₂₀-alkyl radical,

R¹⁴ stands for hydrogen or a protective group PG¹⁴,

R¹⁵, R¹⁶ are the same or different and stand for hydrogen, a C₁-C₂₀-alkyl, aryl, or C₇-C₂₀-aralkyl radical,

A-Y means a group, O—C(═O), O—CH₂, CH₂C(═O), NR¹⁷—C(═O), NR¹⁷—SO₂,

R¹⁷ means hydrogen, or C₁-C₁₀-alkyl,

Z means an oxygen atom or H/OR¹⁸,

whereby

R¹⁸ means hydrogen or a protective group PG¹⁸,

R⁸ means OH or OPG⁸,

Hal means halogen, preferably fluorine, chlorine or bromine, excluding those compounds in which R^{2a} is hydrogen, and R^{2b} means hydrogen, alkyl or aryl, and simultaneously

R⁵ means hydrogen, alkyl or aryl, and simultaneously

A-Y means a grouping O—C(═O), O—CH₂, or NR¹⁷-C(═O), and simultaneously

G means a bicyclic or tricyclic aryl radical or a grouping X═(CR¹²)—,

whereby all other radicals can have the indicated meanings.

The compounds that are claimed in WO 99/02514 are excluded by the disclaimer.

The compounds that are mentioned below are preferred according to the invention:

(4S,7R,8S,9R,13(Z),16S(Z))-4,8-Dihydroxy-1,10-dioxa-16-(1-fluoro-2-(2-methyl-4-thiazolyl)ethenyl)-5,5,7,9,13-pentamethyl-cyclohexadec-13-ene-2,6-dione

(1S,3S(Z),7S,10R,11S,12R,16R)-7,11-Dihydroxy-3-(1-fluoro-2-(2-methyl-4-thiazolyl)ethenyl)-8,8,10,12,16-pentamethyl-4,13,17-trioxabicyclo[14.1.0]heptadecane-5,9-dione

(4S,7R,8S,9R,13(Z),16S(Z))-4,8-Dihydroxy-1,10-dioxa-7-ethyl-16-(1-fluoro-2-(2-methyl-4-thiazolyl)ethenyl)-5,5,9,13-tetramethyl-cyclohexadec-13-ene-2,6-dione

(1S,3S(Z),7S,10R,11S,12R,16R)-7,11-Dihydroxy-10-ethyl-3-(1-fluoro-2-(2-methyl-4-thiazolyl)ethenyl)-8,8,12,16-tetramethyl-4,13,17-trioxabicyclo[14.1.0]heptadecane-5,9-dione

(4S,7R,8S,9R,13(Z),16S(Z))-4,8-Dihydroxy-1,10-dioxa-16-(1-fluoro-2-(2-methyl-4-thiazolyl)ethenyl)-7-(prop-2-en-1-yl)-5,5,9,13-tetramethyl-cyclohexadec-13-ene-2,6-dione

(1S,3S(Z),7S,10R,11S,12R,16R)-7,11-Dihydroxy-3-(1-fluoro-2-(2-methyl-4-thiazolyl)ethenyl)-10-(prop-2-en-1-yl)-8,8,12,16-tetramethyl-4,13,17-trioxabicyclo[14.1.0]-heptadecane-5,9-dione

(4S,7R,8S,9R,13(Z),16S(Z))-4,8-Dihydroxy-1,10-dioxa-16-(1-fluoro-2-(2-methyloxazol-4-yl)ethenyl)-5,5,7,9,13-pentamethyl-cyclohexadec-13-ene-2,6-dione

(1S,3S(Z),7S,10R,11S,12R,16R)-7,11-Dihydroxy-3-(1-fluoro-2-(2-methyloxazol-4-yl)ethenyl)-8,8,10,12,16-pentamethyl-4,13,17-trioxabicyclo[14.1.0]heptadecane-5,9-dione

(4S,7R,8S,9R,13(Z),16S(Z))-4,8-Dihydroxy-1,10-dioxa-7-ethyl-16-(1-fluoro-2-(2-methyloxazol-4-yl)ethenyl)-5,5,9,13-tetramethyl-cyclohexadec-13-ene-2,6-dione

(1S,3S(Z),7S,10R,11S,12R,16R)-7,11-Dihydroxy-10-ethyl-3-(1-fluoro-2-(2-methyloxazol-4-yl)ethenyl)-8,8,12,16-tetramethyl-4,13,17-trioxabicyclo[14.1.0]heptadecane-5,9-dione

(4S,7R,8S,9R,13(Z),16S(Z))-4,8-Dihydroxy-1,10-dioxa-16-(1-fluoro-2-(2-methyloxazol-4-yl)ethenyl)-7-(prop-2-en-1-yl)-5,5,9,13-tetramethyl-cyclohexadec-13-ene-2,6-dione

(1S,3S(Z),7S,10R,11S,12R,16R)-7,11-Dihydroxy-3-(1-fluoro-2-(2-methyloxazol-4-yl)ethenyl)-10-(prop-2-en-1-yl)-8,8,12,16-tetramethyl-4,13,17-trioxabicyclo[14.1.0]heptadecane-5,9-dione

(4S,7R,8S,9R,13(Z),16S(Z))-4,8-Dihydroxy-1,10-dioxa-16-(1-fluoro-2-(2-pyridyl)ethenyl)-5,5,7,9,13-pentamethyl-cyclohexadec-13-ene-2,6-dione

(1S,3S(Z),7S,10R,11S,12R,16R)-7,11-Dihydroxy-3-(1-fluoro-2-(2-pyridyl)ethenyl)-8,8,10,12,16-pentamethyl-4,13,17-trioxabicyclo[14.1.0]heptadecane-5,9-dione

(4S,7R,8S,9R,13(Z),16S(Z))-4,8-Dihydroxy-1,10-dioxa-7-ethyl-16-(1-fluoro-2-(2-pyridyl)ethenyl)-5,5,9,13-tetramethyl-cyclohexadec-13-ene-2,6-dione

(1S,3S(Z),7S,10R,11S,12R,16R)-7,11-Dihydroxy-10-ethyl-3-(1-fluoro-2-(2-pyridyl)ethenyl)-8,8,12,16-tetramethyl-4,13,17-trioxabicyclo[14.1.0]heptadecane-5,9-dione

(4S,7R,8S,9R,13(Z),16S(Z))-4,8-Dihydroxy-1,10-dioxa-16-(1-fluoro-2-(2-pyridyl)ethenyl)-7-(prop-2-en-1-yl)-5,5,9,13-tetramethyl-cyclohexadec-13-ene-2,6-dione

(1S,3S(Z),7S,10R,11S,12R,16R)-7,11-Dihydroxy-3-(1-fluoro-2-(2-pyridyl)ethenyl)-10-(prop-2-en-1-yl)-8,8,12,16-tetramethyl-4,13,17-trioxabicyclo[14.1.0]heptadecane-5,9-dione

(4S,7R,8S,9R,13(Z),16S(Z))-4,8-Dihydroxy-16-(1-fluoro-2-(2-methyl-4-thiazolyl)ethenyl)-1-aza-10-oxa-5,5,7,9,13-pentamethyl-cyclohexadec-13-ene-2,6-dione

(1S,3S(Z),7S,10R,11S,12R,16R)-7,11-Dihydroxy-3-(1-fluoro-2-(2-methyl-4-thiazolyl)ethenyl)-8,8,10,12,16-pentamethyl-4-aza-13,17-dioxabicyclo[14.1.0]heptadecane-5,9-dione

(4S,7R,8S,9R,13(Z),16S(Z))-4,8-Dihydroxy-7-ethyl-16-(1-fluoro-2-(2-methyl-4-thiazolyl)ethenyl)-1-aza-10-oxa-5,5,9,13-tetramethyl-cyclohexadec-13-ene-2,6-dione

(1S,3S(Z),7S,10R,11S,12R,16R)-7,11-Dihydroxy-10-ethyl-3-(1-fluoro-2-(2-methyl-4-thiazolyl)ethenyl)-8,8,12,16-tetramethyl-4-aza-13,17-dioxabicyclo[14.1.0]heptadecane-5,9-dione

(4S,7R,8S,9R,13(Z),16S(Z))-4,8-Dihydroxy-16-(1-fluoro-2-(2-methyl-4-thiazolyl)ethenyl)-7-(prop-2-en-1-yl)-1-aza-10-oxa-5,5,9,13-tetramethyl-cyclohexadec-13-ene-2,6-dione

(1S,3S(Z),7S,10R,11S,12R,16R)-7,11-Dihydroxy-3-(1-fluoro-2-(2-methyl-4-thiazolyl)ethenyl)-10-(prop-2-en-1-yl)-8,8,12,16-tetramethyl-4-aza-13,17-dioxabicyclo[14.1.0]-heptadecane-5,9-dione

(4S,7R,8S,9R,13(Z),16S(Z))-4,8-Dihydroxy-16-(1-fluoro-2-(2-methyloxazol-4-yl)ethenyl)-1-aza-10-oxa-5,5,7,9,13-pentamethyl-cyclohexadec-13-ene-2,6-dione

(1S,3S(Z),7S,10R,11S,12R,16R)-7,11-Dihydroxy-3-(1-fluoro-2-(2-methyloxazol-4-yl)ethenyl)-8,8,10,12,16-pentamethyl-4-aza-13,17-dioxabicyclo[14.1.0]heptadecane-5,9-dione

(4S,7R,8S,9R,13(Z),16S(Z))-4,8-Dihydroxy-7-ethyl-16-(1-fluoro-2-(2-methyloxazol-4-yl)ethenyl)-1-aza-10-oxa-5,5,9,13-tetramethyl-cyclohexadec-13-ene-2,6-dione

(1S,3S(Z),7S,10R,11S,12R,16R)-7,11-Dihydroxy-1,10-ethyl-3-(1-fluoro-2-(2-methyloxazol-4-yl)ethenyl)-8,8,12,16-tetramethyl-4-aza-13,17-dioxabicyclo[14.1.0]heptadecane-5,9-dione

(4S,7R,8 S,9R,13(Z),16S(Z))-4,8-Dihydroxy-16-(1-fluoro-2-(2-methyloxazol-4-yl)ethenyl)-7-(prop-2-en-1-yl)-1-aza-10-oxa-5,5,9,13-tetramethyl-cyclohexadec-13-ene-2,6-dione

(1S,3S(Z),7S,10R,11S,12R,16R)-7,11-Dihydroxy-3-(1-fluoro-2-(2-methyloxazol-4-yl)ethenyl)-10-(prop-2-en-1-yl)-8,8,12,16-tetramethyl-4-aza-13,17-dioxabicyclo[14.1.0]-heptadecane-5,9-dione

(4S,7R,8S,9R,13(Z),16S(Z))-4,8-Dihydroxy-16-(1-fluoro-2-(2-pyridyl)ethenyl)-1-aza-10-oxa-5,5,7,9,13-pentamethyl-cyclohexadec-13-ene-2,6-dione

(1S,3S(Z),7S,10R,11S,12R,16R)-7,11-Dihydroxy-3-(1-fluoro-2-(2-pyridyl)ethenyl)-8,8,10,12,16-pentamethyl-4-aza-13,17-dioxabicyclo[14.1.0]heptadecane-5,9-dione

(4S,7R,8S,9R,13(Z),16S(Z))-4,8-Dihydroxy-7-ethyl-16-(1-fluoro-2-(2-pyridyl)ethenyl)-1-aza-10-oxa-5,5,9,13-tetramethyl-cyclohexadec-13-ene-2,6-dione

(1S,3S(Z),7S,10R,11S12R,16R)-7,11-Dihydroxy-10-ethyl-3-(1-fluoro-2-(2-pyridyl)ethenyl)-8,8,12,16-tetramethyl-4-aza-13,17-dioxabicyclo[14.1.0]heptadecane-5,9-dione

(4S,7R,8S,9R,13(Z),16S(Z))-4,8-Dihydroxy-16-(1-fluoro-2-(2-pyridyl)ethenyl)-7-(prop-2-en-1-yl)-1-aza-10-oxa-5,5,9,13-tetramethyl-cyclohexadec-13-ene-2,6-dione

(1S,3S(Z),7S,10R,11S,12R,16R)-7,11-Dihydroxy-3-(1-fluoro-2-(2-pyridyl)ethenyl)-10-(prop-2-en-1-yl)-8,8,12,16-tetramethyl-4-aza-13,17-dioxabicyclo[14.1.0]heptadecane-5,9-dione

(4S,7R,8S,9R,13(Z),16S(Z))-4,8-Dihydroxy-1,10-dioxa-16-(1-chloro-2-(2-methyl-4-thiazolyl)ethenyl)-5,5,7,9,13-pentamethyl-cyclohexadec-13-ene-2,6-dione

(1S,3S(Z),7S,10R,11S,12R,16R)-7,11-Dihydroxy-3-(1-chloro-2-(2-methyl-4-thiazolyl)ethenyl)-8,8,10,12,16-pentamethyl-4,13,17-trioxabicyclo[14.1.0]heptadecane-5,9-dione

(4S,7R,8S,9R,13(Z),16S(Z))-4,8-Dihydroxy-1,10-dioxa-7-ethyl-16-(1-chloro-2-(2-methyl-4-thiazolyl)ethenyl)-5,5,9,13-tetramethyl-cyclohexadec-13-ene-2,6-dione

(1S,3S(Z),7S,10R,11S,12R,16R)-7,11-Dihydroxy-10-ethyl-3-(1-chloro-2-(2-methyl-4-thiazolyl)ethenyl)-8,8,12,16-tetramethyl-4,13,17-trioxabicyclo[14.1.0]heptadecane-5,9-dione

(4S,7R,8S,9R,13(Z),16S(Z))-4,8-Dihydroxy-1,10-dioxa-16-(1-chloro-2-(2-methyl-4-thiazolyl)ethenyl)-7-(prop-2-en-1-yl)-5,5,9,13-tetramethyl-cyclohexadec-13-ene-2,6-dione

(1S,3S(Z),7S,10R,11S,12R,16R)-7,11-Dihydroxy-3-(1-chloro-2-(2-methyl-4-thiazolyl)ethenyl)-10-(prop-2-en-1-yl)-8,8,12,16-tetramethyl-4,13,17-trioxabicyclo[14.1.0]-heptadecane-5,9-dione

(4S,7R,8S,9R,13(Z),16S(Z))-4,8-Dihydroxy-1,10-dioxa-16-(1-chloro-2-(2-methyloxazol-4-yl)ethenyl)-5,5,7,9,13-pentamethyl-cyclohexadec-13-ene-2,6-dione

(1S,3S(Z),7S,10R,11S,12R,16R)-7,11-Dihydroxy-3-(1-chloro-2-(2-methyloxazol-4-yl)ethenyl)-8,8,10,12,16-pentamethyl-4,13,17-trioxabicyclo[14.1.0]heptadecane-5,9-dione

(4S,7R,8S,9R,13(Z),16S(Z))-4,8-Dihydroxy-1,10-dioxa-7-ethyl-16-(1-chloro-2-(2-methyloxazol-4-yl)ethenyl)-5,5,9,13-tetramethyl-cyclohexadec-13-ene-2,6-dione

(1S,3S(Z),7S,10R,11S,12R,16R)-7,11-Dihydroxy-10-ethyl-3-(1-chloro-2-(2-methyloxazol-4-yl)ethenyl)-8,8,12,16-tetramethyl-4,13,17-trioxabicyclo[14.1.0]heptadecane-5,9-dione

(4S,7R,8S,9R,13(Z),16S(Z))-4,8-Dihydroxy-1,10-dioxa-16-(1-chloro-2-(2-methyloxazol-4-yl)ethenyl)-7-(prop-2-en-1-yl)-5,5,9,13-tetramethyl-cyclohexadec-13-ene-2,6-dione

(1S,3S(Z),7S,10R,11S,12R,16R)-7,11-Dihydroxy-3-(1-chloro-2-(2-methyloxazol-4-yl)ethenyl)-10-(prop-2-en-1-yl)-8,8,12,16-tetramethyl-4,13,17-trioxabicyclo[14.1.0]heptadecane-5,9-dione

(4S,7R,8S,9R,13(Z),16S(Z))-4,8-Dihydroxy-1,10-dioxa-16-(1-chloro-2-(2-pyridyl)ethenyl)-5,5,7,9,13-pentamethyl-cyclohexadec-13-ene-2,6-dione

(1S,3S(Z),7S,10R,11S,12R,16R)-7,11-Dihydroxy-3-(1-chloro-2-(2-pyridyl)ethenyl)-8,8,10,12,16-pentamethyl-4,13,17-trioxabicyclo[14.1.0]heptadecane-5,9-dione

(4S,7R,8S,9R,13(Z),16S(Z))-4,8-Dihydroxy-1,10-dioxa-7-ethyl-16-(1-chloro-2-(2-pyridyl)ethenyl)-5,5,9,13-tetramethyl-cyclohexadec-13-ene-2,6-dione

(1S,3S(Z),7S,10R,11S,12R,16R)-7,11-Dihydroxy-10-ethyl-3-(1-chloro-2-(2-pyridyl)ethenyl)-8,8,12,16-tetramethyl-4,13,17-trioxabicyclo[14.1.0]heptadecane-5,9-dione

(4S,7R,8S,9R,13(Z),16S(Z))-4,8-Dihydroxy-1,10-dioxa-16-(1-chloro-2-(2-pyridyl)ethenyl)-7-(prop-2-en-1-yl)-5,5,9,13-tetramethyl-cyclohexadec-13-ene-2,6-dione

(1S,3S(Z),7S,10R,11S,12R,16R)-7,11-Dihydroxy-3-(1-chloro-2-(2-pyridyl)ethenyl)-10-(prop-2-en-1-yl)-8,8,12,16-tetramethyl-4,13,17-trioxabicyclo[14.1.0]heptadecane-5,9-dione

(4S,7R,8S,9R,13(Z),16S(Z))-4,8-Dihydroxy-16-(1-chloro-2-(2-methyl-4-thiazolyl)ethenyl)-1-aza-10-oxa-5,5,7,9,13-pentamethyl-cyclohexadec-13-ene-2,6-dione

(1S,3S(Z),7S,10R,11S,12R,16R)-7,11-Dihydroxy-3-(1-chloro-2-(2-methyl-4-thiazolyl)ethenyl)-8,8,10,12,16-pentamethyl-4-aza-13,17-dioxabicyclo[14.1.0]heptadecane-5,9-dione

(4S,7R,8S,9R,13(Z),16S(Z))-4,8-Dihydroxy-7-ethyl-16-(1-chloro-2-(2-methyl-4-thiazolyl)ethenyl)-1-aza-10-oxa-5,5,9,13-tetramethyl-cyclohexadec-13-ene-2,6-dione

(1S,3S(Z),7S,10R,11S,12R,16R)-7,11-Dihydroxy-10-ethyl-3-(1-chloro-2-(2-methyl-4-thiazolyl)ethenyl)-8,8,12,16-tetramethyl-4-aza-13,17-dioxabicyclo[14.1.0]heptadecane-5,9-dione

(4S,7R,8S,9R,13(Z),16S(Z))-4,8-Dihydroxy-16-(1-chloro-2-(2-methyl-4-thiazolyl)ethenyl)-7-(prop-2-en-1-yl)-1-aza-10-oxa-5,5,9,13-tetramethyl-cyclohexadec-13-ene-2,6-dione

(1S,3S(Z),7S,10R,11S,12R,16R)-7,11-Dihydroxy-3-(1-chloro-2-(2-methyl-4-thiazolyl)ethenyl)-10-(prop-2-en-1-yl)-8,8,12,16-tetramethyl-4-aza-13,17-dioxabicyclo[14.1.0]-heptadecane-5,9-dione

(4S,7R,8S,9R,13(Z),16S(Z))-4,8-Dihydroxy-16-(1-chloro-2-(2-methyloxazol-4-yl)ethenyl)-1-aza-10-oxa-5,5,7,9,13-pentamethyl-cyclohexadec-13-ene-2,6-dione

(1S,3S(Z),7S,10R,11S,12R,16R)-7,11-Dihydroxy-3-(1-chloro-2-(2-methyloxazol-4-yl)ethenyl)-8,8,10,12,16-pentamethyl-4-aza-13,17-dioxabicyclo[14.1.0]heptadecane-5,9-dione

(4S,7R,8S,9R,13(Z),16S(Z))-4,8-Dihydroxy-7-ethyl-16-(1-chloro-2-(2-methyloxazol-4-yl)ethenyl)-1-aza-10-oxa-5,5,9,13-tetramethyl-cyclohexadec-13-ene-2,6-dione

(1S,3S(Z),7S,10R,11S,12R,16R)-7,11-Dihydroxy-10-ethyl-3-(1-chloro-2-(2-methyloxazol-4-yl)ethenyl)-8,8,12,16-tetramethyl-4-aza-13,17-dioxabicyclo[14.1.0]heptadecane-5,9-dione

(4S,7R,8S,9R,13(Z),16S(Z))-4,8-Dihydroxy-16-(1-chloro-2-(2-methyloxazol-4-yl)ethenyl)-7-(prop-2-en-1-yl)-1-aza-10-oxa-5,5,9,13-tetramethyl-cyclohexadec-13-ene-2,6-dione

(1S,3S(Z),7S,10R,11S,12R,16R)-7,11-Dihydroxy-3-(1-chloro-2-(2-methyloxazol-4-yl)ethenyl)-10-(prop-2-en-1-yl)-8,8,12,16-tetramethyl-4-aza-13,17-dioxabicyclo[14.1.0]heptadecane-5,9-dione

(4S,7R,8S,9R,13(Z),16S(Z))-4,8-Dihydroxy-16-(1-chloro-2-(2-pyridyl)ethenyl)-1-aza-10-oxa-5,5,7,9,13-pentamethyl-cyclohexadec-13-ene-2,6-dione

(1S,3S(Z),7S,10R,11S,12R,16R)-7,11-Dihydroxy-3-(1-chloro-2-(2-pyridyl)ethenyl)-8,8,10,12,16-pentamethyl-4-aza-13,17-dioxabicyclo[14.1.0]heptadecane-5,9-dione

(4S,7R,8S,9R,13(Z),16S(Z))-4,8-Dihydroxy-7-ethyl-16-(1-chloro-2-(2-pyridyl)ethenyl)-1-aza-10-oxa-5,5,9,13-tetramethyl-cyclohexadec-13-ene-2,6-dione

(1S,3S(Z),7S,10R,11S,12R,16R)-7,11-Dihydroxy-10-ethyl-3-(1-chloro-2-(2-pyridyl)ethenyl)-8,8,12,16-tetramethyl-4-aza-13,17-dioxabicyclo[14.1.0]heptadecane-5,9-dione

(4S,7R,8S,9R,13(Z),16S(Z))-4,8-Dihydroxy-16-(1-chloro-2-(2-pyridyl)ethenyl)-7-(prop-2-en-1-yl)-1-aza-10-oxa-5,5,9,13-tetramethyl-cyclohexadec-13-ene-2,6-dione

(1S,3S(Z),7S,10R,11S,12R,16R)-7,11-Dihydroxy-3-(1-chloro-2-(2-pyridyl)ethenyl)-10-(prop-2-en-1-yl)-8,8,12,16-tetramethyl-4-aza-13,17-dioxabicyclo[14.1.0]heptadecane-5,9-dione

(4S,7R,8S,9R,13(Z),16S(Z))-4,8-Dihydroxy-1,10-dioxa-16-(1-fluoro-2-(2-methyl-4-thiazolyl)ethenyl)-7,9,13-trimethyl-5,5-(1,3-trimethylene)cyclohexadec-13-ene-2,6-dione

(1S,3S(Z),7S,10R,11S,12R,16R)-7,11-Dihydroxy-3-(1-fluoro-2-(2-methyl-4-thiazolyl)ethenyl)-10,12,16-trimethyl-8,8-(1,3-trimethylene)-4,13,17-trioxabicyclo[14.1.0]hepta-deca-5,9-dione

(4S,7R,8S,9R,13(Z),16S(Z))-4,8-Dihydroxy-1,10-dioxa-9,13-dimethyl-7-ethyl-16-(1-fluoro-2-(2-methyl-4-thiazolyl)ethenyl)-5,5-(1,3-trimethylene)cyclohexadec-13-ene-2,6-dione

(1S,3S(Z),7S,10R,11S,12R,16R)-7,11-Dihydroxy-12,16-dimethyl-10-ethyl-3-(1-fluoro-2-(2-methyl-4-thiazolyl)ethenyl)-8,8-(1,3-trimethylene)-4,13,17-trioxabicyclo[14.1.0]hepta-decane-5,9-dione

(4S,7R,8S,9R,13(Z),16S(Z))-4,8-Dihydroxy-1,10-dioxa-9,13-dimethyl-16-(1-fluoro-2-(2-methyl-4-thiazolyl)ethenyl)-7-(prop-2-en-1-yl)-5,5-(1,3-trimethylene)cyclohexadec-13-ene-2,6-dione

(1S,3S(Z),7S,10R,1S,12R,16R)-7,11-Dihydroxy-12,16-dimethyl-3-(1-fluoro-2-(2-methyl-4-thiazolyl)ethenyl)-10-(prop-2-en-1-yl)-8,8-(1,3-trimethylene)-4,13,17-trioxabicyclo[14.1.0]hepta-decane-5,9-dione

4S,7R,8S,9R,13(Z),16S(Z))-4,8-Dihydroxy-16-(1-fluoro-2-(2-methyl-4-thiazolyl)ethenyl)-1-aza-10-oxa-7,9,13-trimethyl-5,5-(1,3-trimethylene)cyclohexadec-13-ene-2,6-dione

(1S,3S(Z),7S,10R,11S,12R,16R)-7,11-Dihydroxy-3-(1-fluoro-2-(2-methyl-4-thiazolyl)ethenyl)-10,12,16-trimethyl-8,8-(1,3-trimethylene)-4-aza-13,17-dioxabicyclo[14.1.0]hepta-deca-5,9-dione

(4S,7R,8S,9R,13(Z),16S(Z))-4,8-Dihydroxy-9,13-dimethyl-7-ethyl-16-(1-fluoro-2-(2-methyl-4-thiazolyl)ethenyl)-1-aza-10-oxa-5,5-(1,3-trimethylene)cyclohexadec-13-ene-2,6-dione

(1S,3S(Z),7S,10R,11S,12R,16R)-7,1-Dihydroxy-12,16-dimethyl-10-ethyl-3-(1-fluoro-2-(2-methyl-4-thiazolyl)ethenyl)-8,8-(1,3-trimethylene)-4-aza-13,17-dioxabicyclo[14.1.0]hepta-decane-5,9-dione

(4S,7R,8S,9R,13(Z),16S(Z))-4,8-Dihydroxy-9,13-dimethyl-16-(1-fluoro-2-(2-methyl-4-thiazolyl)ethenyl)-7-(prop-2-en-1-yl)-1-aza-10-oxa-5,5-(1,3-trimethylene)cyclohexadec-13-ene-2,6-dione

(1S,3S(Z),7S,10R,11S,12R,16R)-7,11-Dihydroxy-12,16-dimethyl-3-(1-fluoro-2-(2-methyl-4-thiazolyl)ethenyl)-10-(prop-2-en-1-yl)-8,8-(1,3-trimethylene)-4-aza-13,17-dioxabicyclo[14.1.0]hepta-decane-5,9-dione

(4S,7R,8S,9R,13(Z),16S(Z))-4,8-Dihydroxy-1,10-dioxa-16-(1-chloro-2-(2-methyl-4-thiazolyl)ethenyl)-7,9,13-trimethyl-5,5-(1,3-trimethylene)cyclohexadec-13-ene-2,6-dione

(1S,3S(Z),7S,10R,11S,12R,16R)-7,11-Dihydroxy-3-(1-chloro-2-(2-methyl-4-thiazolyl)ethenyl)-10,12,16-trimethyl-8,8-(1,3-trimethylene)-4,13,17-trioxabicyclo[14.1.0]hepta-deca-5,9-dione

(4S,7R,8S,9R,13(Z),16S(Z))-4,8-Dihydroxy-1,10-dioxa-9,13-dimethyl-7-ethyl-16-(1-chloro-2-(2-methyl-4-thiazolyl)ethenyl)-5,5-(1,3-trimethylene)cyclohexadec-13-ene-2,6-dione

(1S,3S(Z),7S,10R,11S,12R,16R)-7,11-Dihydroxy-12,16-dimethyl-10-ethyl-3-(1-chloro-2-(2-methyl-4-thiazolyl)ethenyl)-8,8-(1,3-trimethylene)-4,13,17-trioxabicyclo[14.1.0]hepta-decane-5,9-dione

(4S,7R,8S,9R,13(Z),16S(Z))-4,8-Dihydroxyl-10-dioxa-9,13-dimethyl-16-(1-chloro-2-(2-methyl-4-thiazolyl)ethenyl)-7-(prop-2-en-1-yl)-5,5-(1,3-trimethylene)cyclohexadec-13-ene-2,6-dione

(1S,3S(Z),7S,10R,11S,12R,16R)-7,11-Dihydroxy-12,16-dimethyl-3-(1-chloro-2-(2-methyl-4-thiazolyl)ethenyl)-10-(prop-2-en-1-yl)-8,8-(1,3-trimethylene)-4,13,17-trioxabicyclo[14.1.0]hepta-decane-5,9-dione

4S,7R,8S,9R,13(Z),16S(Z))-4,8-Dihydroxy-16-(1-chloro-2-(2-methyl-4-thiazolyl)ethenyl)-1-aza-10-oxa-7,9,13-trimethyl-5,5-(1,3-trimethylene)cyclohexadec-13-ene-2,6-dione

(1S,3S(Z),7S,10R,11S,12R,16R)-7,11-Dihydroxy-3-(1-chloro-2-(2-methyl-4-thiazolyl)ethenyl)-10,12,16-trimethyl-8,8-(1,3-trimethylene)-4-aza-13,17-dioxabicyclo[14.1.0]-hepta-deca-5,9-dione

(4S,7R,8S,9R,13(Z),16S(Z))-4,8-Dihydroxy-9,13-dimethyl-7-ethyl-16-(1-chloro-2-(2-methyl-4-thiazolyl)ethenyl)-1-aza-10-oxa-5,5-(1,3-trimethylene)cyclohexadec-13-ene-2,6-dione

(1S,3S(Z),7S,10R,11S,12R,16R)-7,11-Dihydroxy-12,16-dimethyl-10-ethyl-3-(1-chloro-2-(2-methyl-4-thiazolyl)ethenyl)-8,8-(1,3-trimethylene)-4-aza-13,17-dioxabicyclo[14.1.0]hepta-decane-5,9-dione

(4S,7R,8S,9R,13(Z),16S(Z))-4,8-Dihydroxy-9,13-dimethyl-16-(1-chloro-2-(2-methy1-4-thiazolyl)ethenyl)-7-(prop-2-en-1-yl)-1-aza-10-oxa-5,5-(1,3-trimethylene)cyclohexadec-13-ene-2,6-dione

(1S,3S(Z),7S,10R,11S,12R,16R)-7,11-Dihydroxy-12,16-dimethyl-3-(1-chloro-2-(2-methyl-4-thiazolyl)ethenyl)-10-(prop-2-en-1-yl)-8,8-(1,3-trimethylene)-4-aza-13,17-dioxabicyclo[14.1.0]hepta-decane-5,9-dione

(4S,7R,8S,9R,13(Z),16S(Z))-4,8-Dihydroxy-1,10-dioxa-16-(1-methyl-2-(2-methyl-4-thiazolyl)ethenyl)-7-(prop-2-en-1-yl)-5,5,9,13-tetramethyl-cyclohexadec-13-ene-2,6-dione

(1S,3S(Z),7S,10R,11S,12R,16R)-7,11-Dihydroxy-3-(1-methyl-2-(2-methyl-4-thiazolyl)ethenyl)-10-(prop-2-en-1-yl)-8,8,12,16-tetramethyl-4,13,17-trioxabicyclo-[14.1.0]heptadecane-5,9-dione

(4S,7R,8S,9R,13(Z),16S(Z))-4,8-Dihydroxy-1,10-dioxa-16-(1-methyl-2-(2-methyloxazol-4-yl)ethenyl)-7-(prop-2-en-1-yl)-5,5,9,13-tetramethyl-cyclohexadec-13-ene-2,6-dione

(1S,3S(Z),7S,10R,11S,12R,16R)-7,11-Dihydroxy-3-(1-methyl-2-(2-methyloxazol-4-yl)ethenyl)-10-(prop-2-en-1-yl)-8,8,12,16-tetramethyl-4,13,17-trioxabicyclo[14.1.0]heptadecane-5,9-dione

(4S,7R,8S,9R,13(Z),16S(Z))-4,8-Dihydroxy-1,10-dioxa-16-(1-methyl-2-(2-pyridyl)ethenyl)-7-(prop-2-en-1-yl)-5,5,9,13-tetramethyl-cyclohexadec-13-ene-2,6-dione

(1S,3S(Z),7S,10R,11S,12R,16R)-7,11-Dihydroxy-3-(1-methyl-2-(2-pyridyl)ethenyl)-10-(prop-2-en-1-yl)-8,8,12,16-tetramethyl-4,13,17-trioxabicyclo[14.1.0]heptadecane-5,9-dione

(4S,7R,8 S,9R,13(Z),16S(Z))-4,8-Dihydroxy-16-(1-methyl-2-(2-methyl-4-thiazolyl)ethenyl)-7-(prop-2-en-1-yl)-1-aza-10-oxa-5,5,9,13-tetramethyl-cyclohexadec-13-ene-2,6-dione

(1S,3S(Z),7S,10R,11S,12R,16R)-7,11-Dihydroxy-3-(1-methyl-2-(2-methyl-4-thiazolyl)ethenyl)-10-(prop-2-en-1-yl)-8,8,12,16-tetramethyl-4-aza-13,17-dioxabicyclo[14.1.0]-heptadecane-5,9-dione

(4S,7R,8S,9R,13(Z),16S(Z))-4,8-Dihydroxy-16-(1-methyl-2-(2-methyloxazol-4-yl)ethenyl)-7-(prop-2-en-1-yl)-1-aza-10-oxa-5,5,9,13-tetramethyl-cyclohexadec-13-ene-2,6-dione

(1S,3S(Z),7S,10R,11S,12R,16R)-7,11-Dihydroxy-3-(1-methyl-2-(2-methyloxazol-4-yl)ethenyl)-10-(prop-2-en-1-yl)-8,8,12,16-tetramethyl-4-aza-13,17-dioxabicyclo[14.1.0]heptadecane-5,9-dione

(4S,7R,8S,9R,13(Z),16S(Z))-4,8-Dihydroxy-16-(1-methyl-2-(2-pyridyl)ethenyl)-7-(prop-2-en-1-yl)-1-aza-10-oxa-5,5,9,13-tetramethyl-cyclohexadec-13-ene-2,6-dione

(1S,3S(Z),7S,10R,11S,12R,16R)-7,11-Dihydroxy-3-(1-methyl-2-(2-pyridyl)ethenyl)-10-(prop-2-en-1-yl)-8,8,12,16-tetramethyl-4-aza-13,17-dioxabicyclo[14.1. O]heptadecane-5,9-dione

(4S,7R,8S,9R,13(Z),16S(Z))-4,8-Dihydroxy-1,10-dioxa-9,13-dimethyl-16-(1-methyl-2-(2-methyl-4-thiazolyl)ethenyl)-7-(prop-2-en-1-yl)-5,5-(1,3-trimethylene)cyclohexadec-13-ene-2,6-dione

(1S,3S(Z),7S,10R,11S,12R,16R)-7,11-Dihydroxy-12,16-dimethyl-3-(1-methyl-2-(2-methyl-4-thiazolyl)ethenyl)-10-(prop-2-en-1-yl)-8,8-(1,3-trimethylene)-4,13,17-trioxabicyclo[14.1.0]hepta-decane-5,9-dione

(4S,7R,8S,9R,13(Z),16S(Z))-4,8-Dihydroxy-9,13-dimethyl-16-(1-methyl-2-(2-methyl-4-thiazolyl)ethenyl)-7-(prop-2-en-1-yl)-1-aza-10-oxa-5,5-(1,3-trimethylene)cyclohexadec-13-ene-2,6-dione

(1S,3S(Z),7S,10R,11S,12R,16R)-7,11-Dihydroxy-12,16-dimethyl-3-(1-methyl-2-(2-methyl-4-thiazolyl)ethenyl)-10-(prop-2-en-1-yl)-8,8-(1,3-trimethylene)-4-aza-13,17-dioxabicyclo[14.1.0]hepta-decane-5,9-dione

(4S,7R,8S,9R,13(Z),16S(Z))-4,8-Dihydroxy-1,10-dioxa-16-(1-methyl-2-(2-methyl-4-thiazolyl)ethenyl)-7-(prop-2-in-1-yl)-5,5,9,13-tetramethyl-cyclohexadec-13-ene-2,6-dione

(1S,3S(Z),7S,10R,11S,12R,16R)-7,11-Dihydroxy-3-(1-methyl-2-(2-methyl-4-thiazolyl)ethenyl)-10-(prop-2-in-1-yl)-8,8,12,16-tetramethyl-4,13,17-trioxabicyclo[14.1.0]heptadecane-5,9-dione

(4S,7R,8S,9R,13(Z),16S(Z))-4,8-Dihydroxy-1,10-dioxa-16-(1-methyl-2-(2-methyloxazol-4-yl)ethenyl)-7-(prop-2-in-1-yl)-5,5,9,13-tetramethyl-cyclohexadec-13-ene-2,6-dione

(1S,3S(Z),7S,10R,11S,12R,16R)-7,11-Dihydroxy-3-(1-methyl-2-(2-methyloxazol-4-yl)ethenyl)-10-(prop-2-in-1-yl)-8,8,12,16-tetramethyl-4,13,17-trioxabicyclo[14.1.0]heptadecane-5,9-dione

(4S,7R,8S,9R,13(Z),16S(Z))-4,8-Dihydroxy-1,10-dioxa-16-(1-methyl-2-(2-pyridyl)ethenyl)-7-(prop-2-in-1-yl)-5,5,9,13-tetramethyl-cyclohexadec-13-ene-2,6-dione

(1S,3S(Z),7S,10R,11S,12R,16R)-7,11-Dihydroxy-3-(1-methyl-2-(2-pyridyl)ethenyl)-10-(prop-2-in-1-yl)-8,8,12,16-tetramethyl-4,13,17-trioxabicyclo[14.1.0]heptadecane-5,9-dione

(4S,7R,8S,9R,13(Z),16S(Z))-4,8-Dihydroxy-16-(1-methyl-2-(2-methyl-4-thiazolyl)ethenyl)-7-(prop-2-in-1-yl)-1-aza-10-oxa-5,5,9,13-tetramethyl-cyclohexadec-13-ene-2,6-dione

(1S,3S(Z),7S,10R,11S,12R,16R)-7,11-Dihydroxy-3-(1-methyl-2-(2-methyl-4-thiazolyl)ethenyl)-10-(prop-2-in-1-yl)-8,8,12,16-tetramethyl-4-aza-13,17-dioxabicyclo[14.1.0]heptadecane-5,9-dione

(4S,7R,8S,9R,13(Z),16S(Z))-4,8-Dihydroxy-16-(1-methyl-2-(2-methyloxazol-4-yl)ethenyl)-7-(prop-2-in-1-yl)-1-aza-10-oxa-5,5,9,13-tetramethyl-cyclohexadec-13-ene-2,6-dione

(1S,3S(Z),7S,10R,11S,12R,16R)-7,11-Dihydroxy-3-(1-methyl-2-(2-methyloxazol-4-yl)ethenyl)-10-(prop-2-in-1-yl)-8,8,12,16-tetramethyl-4-aza-13,17-dioxabicyclo[14.1.0]-heptadecane-5,9-dione

(4S,7R,8S,9R,13(Z),16S(Z))-4,8-Dihydroxy-16-(1-methyl-2-(2-pyridyl)ethenyl)-7-(prop-2-in-1-yl)-1-aza-10-oxa-5,5,9, 13-tetramethyl-cyclohexadec-13-ene-2,6-dione

(1S,3S(Z),7S,10R,11S,12R,16R)-7,11-Dihydroxy-3-(1-methyl-2-(2-pyridyl)ethenyl)-10-(prop-2-in-1-yl)-8,8,12,16-tetramethyl-4-aza-13,17-dioxabicyclo[14.1.0]heptadecane-5,9-dione

(4S,7R,8S,9R,13(Z),16S(Z))-4,8-Dihydroxy-1,10-dioxa-9,13-dimethyl-16-(1-methyl-2-(2-methyl-4-thiazolyl)ethenyl)-7-(prop-2-in-1-yl)-5,5-(1,3-trimethylene)cyclohexadec-13-ene-2,6-dione

(1S,3S(Z),7S,10R,11S,12R,16R)-7,11-Dihydroxy-12,16-dimethyl-3-(1-methyl-2-(2-methyl-4-thiazolyl)ethenyl)-10-(prop-2-in-1-yl)-8,8-(1,3-trimethylene)-4,13,17-trioxabicyclo[14.1.0]hepta-decane-5,9-dione

(4S,7R,8S,9R,13(Z),16S(Z))-4,8-Dihydroxy-9,13-dimethyl-16-(1-methyl-2-(2-methyl-4-thiazolyl)ethenyl)-7-(prop-2-in-1-yl)-1-aza-10-oxa-5,5-(1,3-trimethylene)cyclohexadec-13-ene-2,6-dione

(1S,3S(Z),7S,10R,11S,12R,16R)-7,11-Dihydroxy-12,16-dimethyl-3-(1-methyl-2-(2-methyl-4-thiazolyl)ethenyl)-10-(prop-2-in-1-yl)-8,8-(1,3-trimethylene)-4-aza-13,17-dioxabicyclo[14.1.0]hepta-decane-5,9-dione

(4S,7R,8S,9R,13(Z),16S(Z))-4,8-Dihydroxy-1,10-dioxa-16-(1-methyl-2-(2-methyl-4-thiazolyl)ethenyl)-7-(but-2-en-1-yl)-5,5,9,13-tetramethyl-cyclohexadec-13-ene-2,6-dione

(1S,3S(Z),7S,10R,11S,12R,16R)-7,11-Dihydroxy-3-(1-methyl-2-(2-methyl-4-thiazolyl)ethenyl)-10-(but-2-en-1-yl)-8,8,12,16-tetramethyl-4,13,17-trioxabicyclo[14.1.0]heptadecane-5,9-dione

(4S,7R,8S,9R,13(Z),16S(Z))-4,8-Dihydroxy-1,10-dioxa-16-(1-methyl-2-(2-methyloxazol-4-yl)ethenyl)-7-(but-2-en-1-yl)-5,5,9,13-tetramethyl-cyclohexadec-13-ene-2,6-dione

(1S,3S(Z),7S,10R,11S,12R,16R)-7,11-Dihydroxy-3-(1-methyl-2-(2-methyloxazol-4-yl)ethenyl)-10-(but-2-en-1-yl)-8,8,12,16-tetramethyl-4,13,17-trioxabicyclo[14.1.0]heptadecane-5,9-dione

(4S,7R,8 S,9R,13(Z),16S(Z))-4,8-Dihydroxy-1,10-dioxa-16-(1-methyl-2-(2-pyridyl)ethenyl)-7-(but-2-en-1-yl)-5,5,9,13-tetramethyl-cyclohexadec-13-ene-2,6-dione

(1S,3S(Z),7S,10R,11S,12R,16R)-7,11-Dihydroxy-3-(1-methyl-2-(2-pyridyl)ethenyl)-10-(but-2-en-1-yl)-8,8,12,16-tetramethyl-4,13,17-trioxabicyclo[14.1.0]heptadecane-5,9-dione

(4S,7R,8S,9R,13(Z),16S(Z))-4,8-Dihydroxy-16-(1-methyl-2-(2-methyl-4-thiazolyl)-ethenyl)-7-(but-2-en-1-yl)-1-aza-10-oxa-5,5,9,13-tetramethyl-cyclohexadec-13-ene-2,6-dione

(1S,3S(Z),7S,10R,11S,12R,16R)-7,11-Dihydroxy-3-(1-methyl-2-(2-methyl-4-thiazolyl)ethenyl)-10-(but-2-en-1-yl)-8,8,12,16-tetramethyl-4-aza-13,17-dioxabicyclo[14.1.0]-heptadecane-5,9-dione

(4S,7R,8S,9R,13(Z),16S(Z))-4,8-Dihydroxy-16-(1-methyl-2-(2-methyloxazol-4-yl)ethenyl)-7-(but-2-en-1-yl)-1-aza-10-oxa-5,5,9, 13-tetramethyl-cyclohexadec-13-ene-2,6-dione

(1S,3S(Z),7S,10R,11S,12R,16R)-7,11-Dihydroxy-3-(1-methyl-2-(2-methyloxazol-4-yl)ethenyl)-10-(but-2-en-1-yl)-8,8,12,16-tetramethyl-4-aza-13,17-dioxabicyclo[14.1.0]-heptadecane-5,9-dione

(4S,7R,8S,9R,13(Z),16S(Z))-4,8-Dihydroxy-16-(1-methyl-2-(2-pyridyl)ethenyl)-7-(but-2-en-1-yl)-1-aza-10-oxa-5,5,9,13-tetramethyl-cyclohexadec-13-ene-2,6-dione

(1S,3S(Z),7S,10R,11S,12R,16R)-7,11-Dihydroxy-3-(1-methyl-2-(2-pyridyl)ethenyl)-10-(but-2-en-1-yl)-8,8,12,16-tetramethyl-4-aza-13,17-dioxabicyclo[14.1.0]heptadecane-5,9-dione

(4S,7R,8S,9R,13(Z),16S(Z))-4,8-Dihydroxy-1,10-dioxa-9,13-dimethyl-16-(1-methyl-2-(2-methyl-4-thiazolyl)ethenyl)-7-(but-2-en-1-yl)-5,5-(1,3-trimethylene)cyclohexadec-13-ene-2,6-dione

(1S,3S(Z),7S,10R,11S,12R,16R)-7,11-Dihydroxy-12,16-dimethyl-3-(1-methyl-2-(2-methyl-4-thiazolyl)ethenyl)-10-(but-2-en-1-yl)-8,8-(1,3-trimethylene)-4,13,17-trioxabicyclo[14.1.0]hepta-decane-5,9-dione

(4S,7R,8S,9R,13(Z),16S(Z))-4,8-Dihydroxy-9,13-dimethyl-16-(1-methyl-2-(2-methyl-4-thiazolyl)ethenyl)-7-(but-2-en-1-yl)-1-aza-10-oxa-5,5-(1,3-trimethylene)cyclohexadec-13-ene-2,6-dione

(1S,3S(Z),7S,10R,11S,12R,16R)-7,11-Dihydroxy-12,16-dimethyl-3-(1-methyl-2-(2-methyl-4-thiazolyl)ethenyl)-10-(but-2-en-1-yl)-8,8-(1,3-trimethylene)-4-aza-13,17-dioxabicyclo[14.1.0]hepta-decane-5,9-dione

(4S,7R,8S,9R,13(Z),16S(Z))-4,8-Dihydroxy-1,10-dioxa-16-(1-methyl-2-(2-methyl-4-thiazolyl)ethenyl)-7-(but-2-in-1-yl)-5,5,9,13-tetramethyl-cyclohexadec-13-ene-2,6-dione

(1S,3S(Z),7S,10R,11S,12R,16R)-7, 11-Dihydroxy-3-(1-methyl-2-(2-methyl-4-thiazolyl)ethenyl)-10-(but-2-in-1-yl)-8,8,12,16-tetramethyl-4,13,17-trioxabiyclo[14.1.0]heptadecane-5,9-dione

(4S,7R,8S,9R,13(Z),16S(Z))-4,8-Dihydroxy-1,10-dioxa-16-(1-methyl-2-(2-methyloxazol-4-yl)ethenyl)-7-(but-2-in-1-yl)-5,5,9,13-tetramethyl-cyclohexadec-13-ene-2,6-dione

(1S,3S(Z),7S,10R,11S,12R,16R)-7,11-Dihydroxy-3-(1-methyl-2-(2-methyloxazol-4-yl)ethenyl)-10-(but-2-in-1-yl)-8,8,12,16-tetramethyl-4,13,17-trioxabicyclo[14.1.0]heptadecane-5,9-dione

(4S,7R,8S,9R,13(Z),16S(Z))-4,8-Dihydroxy-1,10-dioxa-16-(1-methyl-2-(2-pyridyl)ethenyl)-7-(but-2-in-1-yl)-5,5,9,13-tetramethyl-cyclohexadec-13-ene-2,6-dione

(1S,3S(Z),7S,10R,11S,12R,16R)-7,11-Dihydroxy-3-(1-methyl-2-(2-pyridyl)ethenyl)-10-(but-2-in-1-yl)-8,8,12,16-tetramethyl-4,13,17-trioxabicyclo[14.1.0]heptadecane-5,9-dione

(4S,7R,8S,9R,13(Z),16S(Z))-4,8-Dihydroxy-16-(1-methyl-2-(2-methyl-4-thiazolyl)ethenyl)-7-(but-2-in-1-yl)-aza-10-oxa-5,5,9,13-tetramethyl-cyclohexadec-13-ene-2,6-dione

(1S,3S(Z),7S,10R,11S,12R,16R)-7,11-Dihydroxy-3-(1-methyl-2-(2-methyl-4-thiazolyl)ethenyl)-10-(but-2-in-1-yl)-8,8,12,16-tetramethyl-4-aza-13,17-dioxabicyclo[14.1.0]-heptadecane-5,9-dione

(4S,7R,8S,9R,13(Z),16S(Z))-4,8-Dihydroxy-16-(1-methyl-2-(2-methyloxazol-4-yl)ethenyl)-7-(but-2-in-1-yl)-1-aza-10-oxa-5,5,9,13-tetramethyl-cyclohexadec-13-ene-2,6-dione

(1S,3S(Z),7S,10R,11S,12R,16R)-7,11-Dihydroxy-3-(1-methyl-2-(2-methyloxazol-4-yl)ethenyl)-10-(but-2-in-1-yl)-8,8,12,16-tetramethyl-4-aza-13,17-dioxabicyclo[14.1.0]-heptadecane-5,9-dione

(4S,7R,8S,9R,13(Z),16S(Z))-4,8-Dihydroxy-16-(1-methyl-2-(2-pyridyl)ethenyl)-7-(but-2-in-1-yl)-1-aza-10-oxa-5,5,9,13-tetramethyl-cyclohexadec-13-ene-2,6-dione

(1S,3S(Z),7S,10R,11S,12R,16R)-7,11-Dihydroxy-3-(1-methyl-2-(2-pyridyl)ethenyl)-10-(but-2-in-1-yl)-8,8,12,16-tetramethyl-4-aza-13,17-dioxabicyclo[14.1.0]heptadecane-5,9-dione

(4S,7R,8S,9R,13(Z),16S(Z))-4,8-Dihydroxy-1,10-dioxa-9,13-dimethyl-16-(1-methyl-2-(2-methyl-4-thiazolyl)ethenyl)-7-(but-2-in-1-yl)-5,5-(1,3-trimethylene)cyclohexadec-13-ene-2,6-dione

(1S,3S(Z),7S,10R,11S,12R,16R)-7,11-Dihydroxy-12,16-dimethyl-3-(1-methyl-2-(2-methyl-4-thiazolyl)ethenyl)-10-(but-2-in-1-yl)-8,8-(1,3-trimethylene)-4,13,17-trioxabicyclo-[14.1.0]hepta-decane-5,9-dione

(4S,7R,8S,9R,13(Z),16S(Z))-4,8-Dihydroxy-9,13-dimethyl-16-(1-methyl-2-(2-methyl-4-thiazolyl)ethenyl)-7-(but-2-in-1-yl)-1-aza-10-oxa-5,5-(1,3-trimethylene)cyclohexadec-13-ene-2,6-dione

(1S,3S(Z),7S,10R,11S,12R,16R)-7,11-Dihydroxy-12,16-dimethyl-3-(1-methyl-2-(2-methyl-4-thiazolyl)ethenyl)-10-(but-2-in-1-yl)-8,8-(1,3-trimethylene)-4-aza-13,17-dioxabicyclo[14.1.0]hepta-decane-5,9-dione

(4S,7R,8S,9R,13(Z),16S(Z))-4,8-Dihydroxy-1,10-dioxa-16-(2-methyl-5-benzoxazolyl)-7-(prop-2-en-1-yl)-5,5,9,13-tetramethyl-cyclohexadec-13-ene-2,6-dione

(1S,3S(Z),7S,10R,11S,12R,16R)-7,11-Dihydroxy-3-(1-methyl-2-(2-methyl-5-benzoxazolyl)-10-(prop-2-en-1-yl)-8,8,12,16-tetramethyl-4,13,17-trioxabicyclo[14.1.0]-heptadecane-5,9-dione

(4S,7R,8S,9R,13(Z),16S(Z))-4,8-Dihydroxy-16-(2-methyl-5-benzoxazolyl)-7-(prop-2-en-1-yl)-1-aza-10-oxa-5,5,9,13-tetramethyl-cyclohexadec-13-ene-2,6-dione

(1S,3S(Z),7S,10R,11S,12R,16R)-7,11-Dihydroxy-3-(2-methyl-5-benzoxazolyl)-10-(prop-2-en-1-yl)-8,8,12,16-tetramethyl-4-aza-13,17-dioxabicyclo[14.1.0]heptadecane-5,9-dione

(4S,7R,8S,9R,13(Z),16S(Z))-4,8-Dihydroxy-1,10-dioxa-9, 13-dimethyl-16-(2-methyl-5-benzoxazolyl)-7-(prop-2-en-1-yl)-5,5-(1,3-trimethylene)cyclohexadec-13-ene-2,6-dione

(1S,3S(Z),7S,10R,11S,12R,16R)-7,11-Dihydroxy-12,16-dimethyl-3-(2-methyl-5-benzoxazolyl)-10-(prop-2-en-1-yl)-8,8-(1,3-trimethylene)-4,13,17-trioxabicyclo[14.1.0]hepta-decane-5,9-dione

(4S,7R,8S,9R,13(Z),16S(Z))-4,8-Dihydroxy-9,13-dimethyl-16-(2-methyl-5-benzoxazolyl-7-(prop-2-en-1-yl)-1-aza-10-oxa-5,5-(1,3-trimethylene)cyclohexadec-13-ene-2,6-dione

(1S,3S(Z),7S,10R,11S,12R,16R)-7,11-Dihydroxy-12,16-dimethyl-3-(2-methyl-5-benzoxazolyl)-10-(prop-2-en-1-yl)-8,8-(1,3-trimethylene)-4-aza-13,17-dioxabicyclo[14.1.0]hepta-decane-5,9-dione

(4S,7R,8S,9R,13(Z),16S(Z))-4,8-Dihydroxy-1,10-dioxa-16-(2-methyl-5-benzoxazolyl)-7-(prop-2-in-1-yl)-5,5,9,13-tetramethyl-cyclohexadec-13-ene-2,6-dione

(1S,3S(Z),7S,10R,11S,12R,16R)-7,11-Dihydroxy-3-(2-methyl-5-benzoxazolyl)-10-(prop-2-in-1-yl)-8,8,12,16-tetramethyl-4,13,17-trioxabicyclo[14.1.0]heptadecane-5,9-dione

(4S,7R,8S,9R,13(Z),16S(Z))-4,8-Dihydroxy-16-(2-methyl-5-benzoxazolyl)-7-(prop-2-in-1-yl)-1-aza-10-oxa-5,5,9,13-tetramethyl-cyclohexadec-13-ene-2,6-dione

(1S,3S(Z),7S,10R,11S,12R,16R)-7,11-Dihydroxy-3-(2-methyl-5-benzoxazolyl)-10-(prop-2-in-1-yl)-8,8,12,16-tetramethyl-4-aza-13,17-dioxabicyclo[14.1.0]heptadecane-5,9-dione

(4S,7R,8S,9R,13(Z),16S(Z))-4,8-Dihydroxy-1,10-dioxa-9, 13-dimethyl-16-(2-methyl-5-benzoxazolyl)-7-(prop-2-in-1-yl)-5,5-(1,3-trimethylene)cyclohexadec-13-ene-2,6-dione

(1S,3S(Z),7S,10R,11S,12R,16R)-7,11-Dihydroxy-12,16-dimethyl-3-(2-methyl-5-benzoxazolyl)-10-(prop-2-in-1-yl)-8,8-(1,3-trimethylene)-4,13,17-trioxabicyclo[14.1.0]hepta-decane-5,9-dione

(4S,7R,8S,9R,13(Z),16S(Z))-4,8-Dihydroxy-9, 13-dimethyl-16-(2-methyl-5-benzoxazolyl)-7-(prop-2-in-1-yl)-1-aza-10-oxa-5,5-(1,3-trimethylene)cyclohexadec-13-ene-2,6-dione

(1S,3S(Z),7S,10R,11S,12R,16R)-7,11-Dihydroxy-12,16-dimethyl-3-(2-methyl-5-benzoxazolyl)-10-(prop-2-in-1-yl)-8,8-(1,3-trimethylene)-4-aza-13,17-dioxabicyclo[14.1.0]hepta-decane-5,9-dione

(4S,7R,8S,9R,13(Z),16S(Z))-4,8-Dihydroxy-1,10-dioxa-16-(2-methyl-5-benzoxazolyl)-7-(but-2-en-1-yl)-5,5,9,13-tetramethyl-cyclohexadec-13-ene-2,6-dione

(1S,3S(Z),7S,10R,11S,12R,16R)-7,11-Dihydroxy-3-(2-methyl-5-benzoxazolyl)-10-(but-2-en-1-yl)-8,8,12,16-tetramethyl-4,13,17-trioxabicyclo[14.1.0]heptadecane-5,9-dione

(4S,7R,8S,9R,13(Z),16S(Z))-4,8-Dihydroxy-16-(2-methyl-5-benzoxazolyl)-7-(but-2-en-1-yl)-1-aza-10-oxa-5,5,9,13-tetramethyl-cyclohexadec-13-ene-2,6-dione

(1S,3S(Z),7S,10R,11S,12R,16R)-7,11-Dihydroxy-3-(2-methyl-5-benzoxazolyl)-10-(but-2-en-1-yl)-8,8,12,16-tetramethyl-4-aza-13,17-dioxabicyclo[14.1.0]heptadecane-5,9-dione

(4S,7R,8S,9R,13(Z),16S(Z))-4,8-Dihydroxy-1,10-dioxa-9,13-dimethyl-16-(2-methyl-5-benzoxazolyl)-7-(but-2-en-1-yl)-5,5-(1,3-trimethylene)cyclohexadec-13-ene-2,6-dione

(1S,3S(Z),7S,10R,11S,12R,16R)-7,11-Dihydroxy-12,16-dimethyl-3-(2-methyl-5-benzoxazolyl)-10-(but-2-en-1′-yl)-8,8-(1,3-trimethylene)-4,13,17-trioxabicyclo[14.1.0]hepta-decane-5,9-dione

(4S,7R,8S,9R,13(Z),16S(Z))-4,8-Dihydroxy-9, 13-dimethyl-16-(2-methyl-5-benzoxazolyl)-7-(but-2-en-1-yl)-1-aza-10-oxa-5,5-(1,3-trimethylene)cyclohexadec-13-ene-2,6-dione

(1S,3S(Z),7S,10R,11S,12R,16R)-7,11-Dihydroxy-12,16-dimethyl-3-(2-methyl-5-benzoxazolyl)-10-(but-2-en-1-yl)-8,8-(1,3-trimethylene)-4-aza-13,17-dioxabicyclo[14.1.0]hepta-decane-5,9-dione

(4S,7R,8S,9R,13(Z),16S(Z))-4,8-Dihydroxy-1,10-dioxa-16-(2-methyl-5-benzoxazolyl)-7-(but-2-in-1-yl)-5,5,9,13-tetramethyl-cyclohexadec-13-ene-2,6-dione

(1S,3S(Z),7S,10R,11S,12R,16R)-7,11-Dihydroxy-3-(2-methyl-5-benzoxazolyl)-10-(but-2-in-1-yl)-8,8,12,16-tetramethyl-4,13,17-trioxabicyclo[14.1.0]heptadecane-5,9-dione

(4S,7R,8S,9R,13(Z),16S(Z))-4,8-Dihydroxy-16-(2-methyl-5-benzoxazolyl)-7-(but-2-in-1-yl)-1-aza-10-oxa-5,5,9,13-tetramethyl-cyclohexadec-13-ene-2,6-dione

(1S,3S(Z),7S,10R,11S,12R,16R)-7,11-Dihydroxy-3-(2-methyl-5-benzoxazolyl)-10-(but-2-in-1-yl)-8,8,12,16-tetramethyl-4-aza-13,17-dioxabicyclo[14.1.0]heptadecane-5,9-dione

(4S,7R,8S,9R,13(Z),16S(Z))-4,8-Dihydroxy-1,10-dioxa-9,13-dimethyl-16-(2-methyl-5-benzoxazolyl)-7-(but-2-in-1-yl)-5,5-(1,3-trimethylene)cyclohexadec-13-ene-2,6-dione

(1S,3S(Z),7S,10R,11S,12R,16R)-7,11-Dihydroxy-12,16-dimethyl-3-(2-methyl-5-benzoxazolyl)-10-(but-2-in-1-yl)-8,8-(1,3-trimethylene)-4,13,17-trioxabicyclo[14.1.0]hepta-decane-5,9-dione

(4S,7R,8S,9R,13(Z),16S(Z))-4,8-Dihydroxy-9,13-dimethyl-16-(2-methyl-5-benzoxazolyl)-7-(but-2-in-1-yl)-1-aza-10-oxa-5,5-(1,3-trimethylene)cyclohexadec-13-ene-2,6-dione

(1S,3S(Z),7S,10R,11S,12R,16R)-7,11-Dihydroxy-12,16-dimethyl-3-(2-methyl-5-benzoxazolyl)-10-(but-2-in-1-yl)-8,8-(1,3-trimethylene)-4-aza-13,17-dioxabicyclo[14.1.0]hepta-decane-5,9-dione

(4S,7R,8S,9R,13(Z),16S(Z))-4,8-Dihydroxy-1,10-dioxa-16-(2-methyl-5-benzothiazolyl)-7-(prop-2-en-1-yl)-5,5,9,13-tetramethyl-cyclohexadec-13-ene-2,6-dione

(1S,3S(Z),7S,10R,11S,12R,16R)-7,11-Dihydroxy-3-(1-methyl-2-(2-methyl-5-benzothiazolyl)-10-(prop-2-en-1-yl)-8,8,12,16-tetramethyl-4,13,17-trioxabicyclo[14.1.0]-heptadecane-5,9-dione

(4S,7R,8S,9R,13(Z),16S(Z))-4,8-Dihydroxy-16-(2-methyl-5-benzothiazolyl)-7-(prop-2-en-1-yl)-1-aza-10-oxa-5,5,9,13-tetramethyl-cyclohexadec-13-ene-2,6-dione

(1S,3S(Z),7S,10R,11S,12R,16R)-7,11-Dihydroxy-3-(2-methyl-5-benzothiazolyl)-10-(prop-2-en-1-yl)-8,8,12,16-tetramethyl-4-aza-13,17-dioxabicyclo[14.1.0]heptadecane-5,9-dione

(4S,7R,8S,9R,13(Z),16S(Z))-4,8-Dihydroxy-1,10-dioxa-9,13-dimethyl-16-(2-methyl-5-benzothiazolyl)-7-(prop-2-en-1-yl)-5,5-(1,3-trimethylene)cyclohexadec-13-ene-2,6-dione

(1S,3S(Z),7S,10R,11S,12R,16R)-7,11-Dihydroxy-12,16-dimethyl-3-(2-methyl-5-benzothiazolyl)-10-(prop-2-en-1-yl)-8,8-(1,3-trimethylene)-4,13,17-trioxabicyclo[14.1.0]hepta-decane-5,9-dione

(4S,7R,8S,9R,13(Z),16S(Z))-4,8-Dihydroxy-9,13-dimethyl-16-(2-methyl-5-benzothiazolyl)-7-(prop-2-en-1-yl)-1-aza-10-oxa-5,5-(1,3-trimethylene)cyclohexadec-13-ene-2,6-dione

(1S,3S(Z),7S,10R,11S,12R,16R)-7,11-Dihydroxy-12,16-dimethyl-3-(2-methyl-5-benzothiazolyl)-10-(prop-2-en-1-yl)-8,8-(1,3-trimethylene)-4-aza-13,17-dioxabicyclo[14.1.0]hepta-decane-5,9-dione

(4S,7R,8S,9R,13(Z),16S(Z))-4,8-Dihydroxy-1,10-dioxa-16-(2-methyl-5-benzothiazolyl)-7-(prop-2-in-1-yl)-5,5,9,13-tetramethyl-cyclohexadec-13-ene-2,6-dione

(1S,3S(Z),7S,10R,11S,12R,16R)-7,11-Dihydroxy-3-(2-methyl-5-benzothiazolyl)-10-(prop-2-in-1-yl)-8,8,12,16-tetramethyl-4,13,17-trioxabicyclo[14.1.0]heptadecane-5,9-dione

(4S,7R,8S,9R,13(Z),16S(Z))-4,8-Dihydroxy-16-(2-methyl-5-benzothiazolyl)-7-(prop-2-in-1-yl)-1-aza-10-oxa-5,5,9,13-tetramethyl-cyclohexadec-13-ene-2,6-dione

(1S,3S(Z),7S,10R,11S,12R,16R)-7,11-Dihydroxy-3-(2-methyl-5-benzothiazolyl)-10-(prop-2-in-1-yl)-8,8,12,16-tetramethyl-4-aza-13,17-dioxabicyclo[14.1.0]heptadecane-5,9-dione

(4S,7R,8S,9R,13(Z),16S(Z))-4,8-Dihydroxy-1,10-dioxa-9,13-dimethyl-16-(2-methyl-5-benzothiazolyl)-7-(prop-2-in-1-yl)-5,5-(1,3-trimethylene)cyclohexadec-13-ene-2,6-dione

(1S,3S(Z),7S,10R,11S,12R,16R)-7,11-Dihydroxy-12,16-dimethyl-3-(2-methyl-5-benzothiazolyl)-10-(prop-2-in-1-yl)-8,8-(1,3-trimethylene)-4,13,17-trioxabicyclo[14.1.0]hepta-decane-5,9-dione

(4S,7R,8S,9R,13(Z),16S(Z))-4,8-Dihydroxy-9,13-dimethyl-16-(2-methyl-5-benzothiazolyl)-7-(prop-2-in-1-yl)-1-aza-10-oxa-5,5-(1,3-trimethylene)cyclohexadec-13-ene-2,6-dione

(1S,3S(Z),7S,10R,11S,12R,16R)-7,11-Dihydroxy-12,16-dimethyl-3-(2-methyl-5-benzothiazolyl)-10-(prop-2-in-1-yl)-8,8-(1,3-trimethylene)-4-aza-13,17-dioxabicyclo[14.1.0]hepta-decane-5,9-dione

(4S,7R,8S,9R,13(Z),16S(Z))-4,8-Dihydroxy-1,10-dioxa-16-(2-methyl-5-benzothiazolyl)-7-(but-2-en-1-yl)-5,5,9, 13-tetramethyl-cyclohexadec-13-ene-2,6-dione

(1S,3S(Z),7S,10R,11S,12R,16R)-7,11-Dihydroxy-3-(2-methyl-5-benzothiazolyl)-10-(but-2-en-1-yl)-8,8,12,16-tetramethyl-4,13,17-, trioxabicyclo[14.1.0]heptadecane-5,9-dione

(4S,7R,8S,9R,13(Z),16S(Z))-4,8-Dihydroxy-16-(2-methyl-5-benzothiazolyl)-7-(but-2-en-1-yl)-1-aza-10-oxa-5,5,9,13-tetramethyl-cyclohexadec-13-ene-2,6-dione

(1S,3S(Z),7S,10R,11S,12R,16R)-7,11-Dihydroxy-3-(2-methyl-5-benzothiazolyl)-10-(but-2-en-1-yl)-8,8,12,16-tetramethyl-4-aza-13,17-dioxabicyclo[14.1.0]heptadecane-5,9-dione

(4S,7R,8S,9R,13(Z),16S(Z))-4,8-Dihydroxy-1,10-dioxa-9,13-dimethyl-16-(2-methyl-5-benzothiazolyl)-7-(but-2-en-1-yl)-5,5-(1,3-trimethylene)cyclohexadec-13-ene-2,6-dione

(1S,3S(Z),7S,10R,11S,12R,16R)-7,11-Dihydroxy-12,16-dimethyl-3-(2-methyl-5-benzothiazolyl)-10-(but-2-en-1-yl)-8,8-(1,3-trimethylene)-4,13,17-trioxabicyclo[14.1.0]hepta-decane-5,9-dione

(4S,7R,8S,9R,13(Z),16S(Z))-4,8-Dihydroxy-9,13-dimethyl-16-(2-methyl-5-benzothiazolyl)-7-(but-2-en-1-yl)-1-aza-10-oxa-5,5-(1,3-trimethylene)cyclohexadec-13-ene-2,6-dione

(1S,3S(Z),7S,10R,11S,12R,16R)-7,11-Dihydroxy-12,16-dimethyl-3-(2-methyl-5-benzothiazolyl)-10-(but-2-en-1-yl)-8,8-(1,3-trimethylene)-4-aza-13,17-dioxabicyclo[14.1.0]hepta-decane-5,9-dione

(4S,7R,8S,9R,13(Z),16S(Z))-4,8-Dihydroxy-1,10-dioxa-16-(2-methyl-5-benzothiazolyl)-7-(but-2-in-1-yl)-5,5,9,13-tetramethyl-cyclohexadec-13-ene-2,6-dione

(1S,3S(Z),7S,10R,11S,12R,16R)-7,11-Dihydroxy-3-(2-methyl-5-benzothiazolyl)-10-(but-2-in-1-yl)-8,8,12,16-tetramethyl-4,13,17-trioxabicyclo[14.1.0]heptadecane-5,9-dione

(4S,7R,8S,9R,13(Z),16S(Z))-4,8-Dihydroxy-16-(2-methyl-5-benzothiazolyl)-7-(but-2-in 1-yl)-1-aza-10-oxa-5,5,9,13-tetramethyl-cyclohexadec-13-ene-2,6-dione

(1S,3S(Z),7S,10R,11S,12R,16R)-7,11-Dihydroxy-3-(2-methyl-5-benzothiazolyl)-10-(but-2-in-1-yl)-8,8,12,16-tetramethyl-4-aza-13,17-dioxabicyclo[14.1.0]heptadecane-5,9-dione

(4S,7R,8S,9R,13(Z),16S(Z))-4,8-Dihydroxy-1,10-dioxa-9,13-dimethyl-16-(2-methyl-5-benzothiazolyl)-7-(but-2-in-1-yl)-5,5-(1,3-trimethylene)cyclohexadec-13-ene-2,6-dione

(1S,3S(Z),7S,10R,11S,12R,16R)-7,11-Dihydroxy-12,16-dimethyl-3-(2-methyl-5-benzothiazolyl)-10-(but-2-in-1-yl)-8,8-(1,3-trimethylene)-4,13,17-trioxabicyclo[14.1.0]hepta-decane-5,9-dione

(4S,7R,8S,9R,13(Z),168(Z))-4,8-Dihydroxy-9,13-dimethyl-16-(2-methyl-5-benzothiazolyl)-7-(but-2-in-1-yl)-1-aza-10-oxa-5,5-(1,3-trimethylene)cyclohexadec-13-ene-2,6-dione

(1S,3S(Z),7S,10R,11S,12R,16R)-7,11-Dihydroxy-12,16-dimethyl-3-(2-methyl-5-benzothiazolyl)-10-(but-2-in-1-yl)-8,8-(1,3-trimethylene)-4-aza-13,17-dioxabicyclo[14.1.0]hepta-decane-5,9-dione

(4S,7R,8S,9S,13E,16S(E))-4,8-Dihydroxy-13-fluoro-16-(1-methyl-2-(2-methyl-4-thiazolyl)ethenyl)-1,10-dioxa-5,5,7,9-tetramethyl-cyclohexadec-13-ene-2,6-dione

(4S,7R,8S,9S,13Z, 16S(E))-4,8-Dihydroxy-13-fluoro-16-(1-methyl-2-(2-methyl-4-thiazolyl)ethenyl)-1,10-dioxa-5,5,7,9-tetramethyl-cyclohexadec-13-ene-2,6-dione

(1S,3S(E),7S,10R,11S,12S,16S)-16-Fluoro-7,11-dihydroxy-3-(1-methyl-2-(2-methyl-4-thiazolyl)ethenyl)-8,8,10,12-tetramethyl-4,13,17-trioxabicyclo[14.1.0]heptadecane-5,9-dione

(1R,3S(E),7S,10R,11S,12S,16R)-16-Fluoro-7,11-dihydroxy-3-(1-methyl-2-(2-methyl-4-thiazolyl)ethenyl)-8,8,10,12-tetramethyl-4,13,17-trioxabicyclo[14.1.0]heptadecane-5,9-dione

(4S,7R,8S,9S,13E,16S(E))-4,8-Dihydroxy-13-chloro-16-(1-methyl-2-(2-methyl-4-thiazolyl)ethenyl)-1,10-dioxa-5,5,7,9-tetraamethyl-cyclohexadec-13-ene-2,6-dione

(4S,7R,8S,9S,13Z,16S(E))-4,8-Dihydroxy-13-chloro-16-(1-methyl-2-(2-methyl-4-thiazolyl)ethenyl)-1,10-dioxa-5,5,7,9-tetramethyl-cyclohexadec-13-ene-2,6-dione

(1S,3S(E),7S,10R,11S,12S,16S)-16-Chloro-7,11-dihydroxy-3-(1-methyl-2-(2-methyl-4-thiazolyl)ethenyl)-8,8,10,12-tetramethyl-4,13,17-trioxabicyclo[14.1.0]heptadecane-5,9-dione

(1R,3S(E),7S,10R,11S,12S,16R)-16-Chloro-7,11-dihydroxy-3-(1-methyl-2-(2-methyl-4-thiazolyl)ethenyl)-8,8,10,12-tetramethyl-4,13,17-trioxabicyclo[14.1.0]heptadecane-5,9-dione

(4S,7R,8S,9S,13E,16S(E))-4,8-Dihydroxy-7-ethyl-13-fluoro-16-(1-methyl-2-(2-methyl-4-thiazolyl)ethenyl)-1,10-dioxa-5,5,9-trimethyl-cyclohexadec-13-ene-2,6-dione

(1S,3S(E),7S,10R,11S,12S,16S)-16-Fluoro-10-ethyl-7,11-dihydroxy-3-(1-methyl-2-(2-methyl-4-thiazolyl)ethenyl)-8,8,12-trimethyl-4,13,17-trioxabicyclo[14.1.0]heptadecane-5,9-dione

(1R,3S(E),7S,10R,11S,12S,16R)-16-Fluoro-10-ethyl-7,11-dihydroxy-3-(1-methyl-2-(2-methyl-4-thiazolyl)ethenyl)-8,8,12-trimethyl-4,13,17-trioxabicyclo[14.1.0]heptadecane-5,9-dione

(4S,7R,8S,9S,13E,16S(E))-4,8-Dihydroxy-7-allyl-13-chloro-16-(1-methyl-2-(2-methyl-4-thiazolyl)ethenyl)-1,10-dioxa-5,5,9-trimethyl-cyclohexadec-13-ene-2,6-dione

(1S,3S(E),7S,10R,11S,12S,16S)-16-Chloro-10-allyl-7,11-dihydroxy-3-(1-methyl-2-(2-methyl-4-thiazolyl)ethenyl)-8,8,12-trimethyl-4,13,17-trioxabicyclo[14.1.0]heptadecane-5,9-dione

(1R,3S(E),7S,10R,11S,12S,16R)-16-Chloro-10-allyl-7,11-dihydroxy-3-(1-methyl-2-(2-methyl-4-thiazolyl)ethenyl)-8,8,12-trimethyl-4,13,17-trioxabicyclo[14.1.0]heptadecane-5,9-dione

(4S,7R,8S,9S,13E,16S(E))-4,8-Dihydroxy-13-chloro-16-(1-methyl-2-(2-methyl-2-pyridyl)ethenyl)-1,10-dioxa-5,5,7,9-tetraamethyl-cyclohexadec-13-ene-2,6-dione

(1S,3S(E),7S,10R,11S,12S,16S)-16-Chloro-7,11-dihydroxy-3-(1-methyl-2-(2-methyl-2-pyridyl)ethenyl)-8,8,10,12-tetramethyl-4,13,17-trioxabicyclo[14.1.0]heptadecane-5,9-dione

(1R,3S(E),7S,10R,11S,12S,16R)-16-Chloro-7,11-dihydroxy-3-(1-methyl-2-(2-methyl-2-pyridyl)ethenyl)-8,8,10,12-tetramethyl-4,13,17-trioxabicyclo[14.1.0]heptadecane-5,9-dione

(4S,7R,8S,9S,13E,16S(Z))-4,8-Dihydroxy-13-chloro-16-(1-fluoro-2-(2-methyl-4-thiazolyl)ethenyl)-1,10-dioxa-5,5,7,9-tetraamethyl-cyclohexadec-13-ene-2,6-dione

(1S,3S(Z),7S,10R,11S,12S,16S)-16-Chloro-7,11-dihydroxy-3-(1-fluoro-2-(2-methyl-4-thiazolyl)ethenyl)-8,8,10,12-tetramethyl-4,13,17-trioxabicyclo[14.1.0]heptadecane-5,9-dione

(1R,3S(Z),7S,10R,11S,12S,16R)-16-Chloro-7,11-dihydroxy-3-(1-fluoro-2-(2-methyl-4-thiazolyl)ethenyl)-8,8,10,12-tetramethyl-4,13,17-trioxabicyclo[14.1.0]heptadecane-5,9-dione

(4S,7R,8S,9S,13E,16S(Z))-4,8-Dihydroxy-13-chloro-16-(1-chloro-2-(2-methyl-4-thiazolyl)ethenyl)-1,10-dioxa-5,5,7,9-tetraamethyl-cyclohexadec-13-ene-2,6-dione

(1S,3S(Z),7S,10R,11S,12S,16S)-16-Chloro-7,11-dihydroxy-3-(1-chloro-2-(2-methyl-4-thiazolyl)ethenyl)-8,8,10,12-tetramethyl-4,13,17-trioxabicyclo[14.1.0]heptadecane-5,9-dione

(1R,3S(Z),7S,10R,11S,12S,16R)-16-Chloro-7,11-dihydroxy-3-(1-chloro-2-(2-methyl-4-thiazolyl)ethenyl)-8,8,10,12-tetramethyl-4,13,17-trioxabicyclo[14.1.0]heptadecane-5,9-dione

(4S,7R,8S,9S,13E,16S(Z))-4,8-Dihydroxy-13-chloro-16-(1-fluoro-2-(2-methyl-4-thiazolyl)ethenyl)-5,5-trimethylene-1,10-dioxa-7,9-dimethyl-cyclohexadec-13-ene-2,6-dione

(1S,3S(Z),7S,10R,11S,12S,16S)-16-Chloro-7,11-dihydroxy-3-(1-fluoro-2-(2-methyl-4-thiazolyl)ethenyl)-8,8-trimethylene-10,12-dimethyl-4,13,17-trioxabicyclo[14.1.0]heptadecane-5,9-dione

(1R,3S(Z),7S,10R,11S,12S,16R)-16-Chloro-7,11-dihydroxy-3-(1-fluoro-2-(2-methyl-4-thiazolyl)ethenyl)-8,8-trimethylene-10,12-dimethyl-4,13,17-trioxabicyclo[14.1.0]heptadecane-5,9-dione

(4S,7R,8S,9S,13E,16S(Z))-4,8-Dihydroxy-13-chloro-16-(1-chloro-2-(2-methyl-4-thiazolyl)ethenyl)-5,5-trimethylene-1,10-dioxa-7,9-dimethyl-cyclohexadec-13-ene-2,6-dione

(1S,3S(Z),7S,10R,11S,12S,16S)-16-Chloro-7,11-dihydroxy-3-(1-chloro-2-(2-methyl-4-thiazolyl)ethenyl)-8,8-trimethylene-10,12-dimethyl-4,13,17-trioxabicyclo[14.1.0]heptadecane-5,9-dione

(1R,3S(Z),7S,10R,11S,12S,16R)-16-Chloro-7,11-dihydroxy-3-(1-chloro-2-(2-methyl-4-thiazolyl)ethenyl)-8,8-trimethylene-10,12-dimethyl-4,13,17-trioxabicyclo[14.1.0]heptadecane-5,9-dione

(4S,7R,8S,9S,13E,16S(Z))-4,8-Dihydroxy-13-chloro-16-(1-fluoro-2-(2-methyl-4-oxazolyl)ethenyl)-1,10-dioxa-5,5,7,9-tetraamethyl-cyclohexadec-13-ene-2,6-dione

(1S,3S(Z),7S,10R,11S,12S,16S)-16-Chloro-7,11-dihydroxy-3-(1-fluoro-2-(2-methyl-4-oxazolyl)ethenyl)-8,8,10,12-tetramethyl-4,13,17-trioxabicyclo[14.1.0]heptadecane-5,9-dione

(1R,3S(Z),7S,10R,11S,12S,16R)-16-Chloro-7,11-dihydroxy-3-(1-fluoro-2-(2-methyl-4-oxazolyl)ethenyl)-8,8,10,12-tetramethyl-4,13,17-trioxabicyclo[14.1.0]heptadecane-5,9-dione

(4S,7R,8S,9S,13E,16S(Z))-4,8-Dihydroxy-13-chloro-16-(1-fluoro-2-(2-methyl-2-pyridyl)ethenyl)-1,10-dioxa-5,5,7,9-tetraamethyl-cyclohexadec-13-ene-2,6-dione

(1S,3S(Z),7S,10R,11S,12S,16S)-16-Chloro-7,11-dihydroxy-3-(1-fluoro-2-(2-methyl-2-pyridyl)ethenyl)-8,8,10,12-tetramethyl-4,13,17-trioxabicyclo[14.1.0]heptadecane-5,9-dione

(1R,3S(Z),7S,10R,1S,12S,16R)-16-Chloro-7,11-dihydroxy-3-(1-fluoro-2-(2-methyl-2-pyridyl)ethenyl)-8,8,10,12-tetramethyl-4,13,17-trioxabicyclo[14.1.0]heptadecane-5,9-dione

(4S,7R,8S,9S,13E,16S(Z))-4,8-Dihydroxy-7-ethyl-13-chloro-16-(1-chloro-2-(2-methyl-2-pyridyl)ethenyl)-1,10-dioxa-5,5,9-trimethyl-cyclohexadec-13-ene-2,6-dione

(1S,3S(Z),7S,10R,11S,12S,16S)-16-Chloro-7,11-dihydroxy-7-ethyl-3-(1-chloro-2-(2-methyl-2-pyridyl)ethenyl)-8,8,12-trimethyl-4,13,17-trioxabicyclo[14.1.0]heptadecane-5,9-dione

(1R,3S(Z),7S,10R,11S,12S,16R)-16-Chloro-7,11-dihydroxy-7-ethyl-3-(1-chloro-2-(2-methyl-2-pyridyl)ethenyl)-8,8,12-trimethyl-4,13,17-trioxabicyclo[14.1.0]heptadecane-5,9-dione

(4S,7R,8S,9S,13E,16S)-4,8-Dihydroxy-13-fluoro-16-(2-methyl-5-benzothiazolyl)-1,10-dioxa-5,5,7,9-tetramethyl-cyclohexadec-13-ene-2,6-dione

(4S,7R,8S,9S,13Z, 16S)-4,8-Dihydroxy-13-fluoro-16-(2-methyl-5-benzothiazolyl)-1,10-dioxa-5,5,7,9-tetramethyl-cyclohexadec-13-ene-2,6-dione

(1S,3S,7S,10R,11S,12S,16S)— 16-Fluoro-7,11-dihydroxy-3-(2-methyl-5-benzothiazolyl)-8,8,10,12-tetramethyl-4,13,17-trioxabicyclo[14.1.0]heptadecane-5,9-dione

(1R,3S,7S,10R,11S,12S,16R)-16-Fluoro-7,11-dihydroxy-3-(2-methyl-5-benzothiazolyl)-8,8,10,12-tetramethyl-4,13,17-trioxabicyclo[14.1.0]heptadecane-5,9-dione

(4S,7R,8S,9S,13E,16S)-4,8-Dihydroxy-13-chloro-16-(2-methyl-5-benzothiazolyl)-1,10-dioxa-5,5,7,9-tetraamethyl-cyclohexadec-13-ene-2,6-dione

(4S,7R,8S,9S,13Z,16S)-4,8-Dihydroxy-13-chloro-16-(2-methyl-5-benzothiazolyl)-1,10-dioxa-5,5,7,9-tetramethyl-cyclohexadec-13-ene-2,6-dione

(1S,3S,7S,10R,11S,12S,16S)— 16-Chloro-7,11-dihydroxy-3-(2-methyl-5-benzothiazolyl)-8,8,10,12-tetramethyl-4,13,17-trioxabicyclo[14.1.0]heptadecane-5,9-dione

(1R,3S,7S,10R,11S,12S,16R)-16-Chloro-7,11-dihydroxy-3-(2-methyl-5-benzothiazolyl)-8,8,10,12-tetramethyl-4,13,17-trioxabicyclo[14.1.0]heptadecane-5,9-dione

(4S,7R,8S,9S,13E,16S)-4,8-Dihydroxy-7-ethyl-13-chloro-16-(2-methyl-5-benzothiazolyl)-1,10-dioxa-5,5,9-trimethyl-cyclohexadec-13-ene-2,6-dione

(1S,3S,7S,10R,11S,12S,16S)-16-Chloro-7,11-dihydroxy-10-ethyl-3-(2-methyl-5-benzothiazolyl)-8,8,12-trimethyl-4,13,17-trioxabicyclo[14.1.0]heptadecane-5,9-dione

(1R,3S,7S,10R,11S,12S,16R)-16-Chloro-7,11-dihydroxy-10-ethyl-3-(2-methyl-5-benzothiazolyl)-8,8, 12-tetramethyl-4,13,17-trioxabicyclo[14.1.0]heptadecane-5,9-dione

(4S,7R,8S,9S,13E,16S)-4,8-Dihydroxy-7-allyl-13-chloro-16-(2-methyl-5-benzothiazolyl)-1,10-dioxa-5,5,9-trimethyl-cyclohexadec-13-ene-2,6-dione

(1S,3S,7S,10R,11S,12S,16S)-16-Chloro-7,11-dihydroxy-10-allyl-3-(2-methyl-5-benzothiazolyl)-8,8,12-trimethyl-4,13,17-trioxabicyclo[14.1.0]heptadecane-5,9-dione

(1R,3S,7S,10R,11S,12S,16R)-16-Chloro-7,11-dihydroxy-10-allyl-3-(2-methyl-5-benzothiazolyl)-8,8, 12-tetramethyl-4,13,17-trioxabicyclo[14.1.0]heptadecane-5,9-dione

(4S,7R,8S,9S,13E,16S)-4,8-Dihydroxy-13-fluoro-16-(2-methyl-5-benzoxazolyl)-1,10-dioxa-5,5,7,9-tetramethyl-cyclohexadec-13-ene-2,6-dione

(4S,7R,8S,9S,13Z, 16S)-4,8-Dihydroxy-13-fluoro-16-(2-methyl-5-benzoxazolyl)-1,10-dioxa-5,5,7,9-tetramethyl-cyclohexadec-13-ene-2,6-dione

(1S,3S,7S,10R,11S,2S,16S)-16-Fluoro-7,11-dihydroxy-3-(2-methyl-5-benzoxazolyl)-8,8,10,12-tetramethyl-4,13,17-trioxabicyclo[14.1.0]heptadecane-5,9-dione

(1R,3S,7S,10R,11S,12S,16R)-16-Fluoro-7,11-dihydroxy-3-(2-methyl-5-benzoxazolyl)-8,8,10,12-tetramethyl-4,13,17-trioxabicyclo[14.1.0]heptadecane-5,9-dione

(4S,7R,8S,9S,13E,16S)-4,8-Dihydroxy-13-chloro-16-(2-methyl-5-benzoxazolyl)-1,10-dioxa-5,5,7,9-tetraamethyl-cyclohexadec-13-ene-2,6-dione

(4S,7R,8S,9S,13Z, 16S)-4,8-Dihydroxy-13-chloro-16-(2-methyl-5-benzoxazolyl)-1,10-dioxa-5,5,7,9-tetramethyl-cyclohexadec-13-ene-2,6-dione

(1S,3S,7S,10R,11S, 12S,16S)— 16-Chloro-7,11-dihydroxy-3-(2-methyl-5-benzoxazolyl)-8,8,10,12-tetramethyl-4,13,17-trioxabicyclo[14.1.0]heptadecane-5,9-dione

(1R,3S,7S,10R,11S,12S,16R)-16-Chloro-7,11-dihydroxy-3-(2-methyl-5-benzothiazolyl)-8,8,10,12-tetramethyl-4,13,17-trioxabicyclo[14.1.0]heptadecane-5,9-dione

(4S,7R,8S,9S,13E,16S(E))-4,8-Dihydroxy-13-fluoro-16-(1-methyl-2-(2-methyl-4-thiazolyl)ethenyl)-1-aza-10-oxa-5,5,7,9-tetramethyl-cyclohexadec-13-ene-2,6-dione

(4S,7R,8S,9S,13Z,16S(E))-4,8-Dihydroxy-13-fluoro-16-(1-methyl-2-(2-methyl-4-thiazolyl)ethenyl)-1-aza-10-oxa-5,5,7,9-tetramethyl-cyclohexadec-13-ene-2,6-dione

(1S,3S(E),7S,10R,11S,12S,16S)-16-Fluoro-7,11-dihydroxy-3-(1-methyl-2-(2-methyl-4-thiazolyl)ethenyl)-8,8,10,12-tetramethyl-4-aza-13,17-dioxabicyclo[14.1.0]heptadecane-5,9-dione

(1R,3S(E),7S,10R,11S,12S,16R)-16-Fluoro-7,11-dihydroxy-3-(1-methyl-2-(2-methyl-4-thiazolyl)ethenyl)-8,8,10,12-tetramethyl-4-aza-13,17-dioxabicyclo[14.1.0]heptadecane-5,9-dione

(4S,7R,8S,9S,13E,16S(E))-4,8-Dihydroxy-13-chloro-16-(1-methyl-2-(2-methyl-4-thiazolyl)ethenyl)-1-aza-10-oxa-5,5,7,9-tetraamethyl-cyclohexadec-13-ene-2,6-dione

(4S,7R,8S,9S,13Z, 16S(E))-4,8-Dihydroxy-13-chloro-16-(1-methyl-2-(2-methyl-4-thiazolyl)ethenyl)-1-aza-10-oxa-5,5,7,9-tetramethyl-cyclohexadec-13-ene-2,6-dione

(1S,3S(E),7S,10R,11S,12S,16S)-16-Chloro-7,11-dihydroxy-3-(1-methyl-2-(2-methyl-4-thiazolyl)ethenyl)-8,8,10,12-tetramethyl-4-aza-13,17-dioxabicyclo[14.1.0]heptadecane-5,9-dione

(1R,3S(E),7S,10R,11S,12S,16R)-16-Chloro-7,11-dihydroxy-3-(1-methyl-2-(2-methyl-4-thiazolyl)ethenyl)-8,8, 10,12-tetramethyl-4-aza-13,17-dioxabicyclo[14.1.0]heptadecane-5,9-dione

(4S,7R,8S,9S,13E,16S(E))-4,8-Dihydroxy-7-ethyl-13-fluoro-16-(1-methyl-2-(2-methyl-4-thiazolyl)ethenyl)-1-aza-10-oxa-5,5,9-trimethyl-cyclohexadec-13-ene-2,6-dione

(1S,3S(E),7S,10R,11S,12S,16S)-16-Fluoro-10-ethyl-7,11-dihydroxy-3-(1-methyl-2-(2-methyl-4-thiazolyl)ethenyl)-8,8,12-trimethyl-4-aza-13,17-dioxabicyclo[14.1.0]heptadecane-5,9-dione

(1R,3S(E),7S,10R,11S,12S,16R)-16-Fluoro-10-ethyl-7,11-dihydroxy-3-(1-methyl-2-(2-methyl-4-thiazolyl)ethenyl)-8,8,12-trimethyl-4-aza-13,17-dioxabicyclo[14.1.0]heptadecane-5,9-dione

(4S,7R,8S,9S,13E,16S(E))-4,8-Dihydroxy-7-allyl-13-chloro-16-(1-methyl-2-(2-methyl-4-thiazolyl)ethenyl)-1-aza-101-ox a-5,5,9-trimethyl-cyclohexadec-13-ene-2,6-dione

(1S,3S(E),7S,10R,11S,12S,16S)-16-Chloro-10-allyl-7,11-dihydroxy-3-(1-methyl-2-(2-methyl-4-thiazolyl)ethenyl)-8,8,12-trimethyl-4-aza-13,17-dioxabicyclo[14.1.0]heptadecane-5,9-dione

(1R,3S(E),7S,10R,11S,12S,16R)-16-Chloro-10-allyl-7,11-dihydroxy-3-(1-methyl-2-(2-methyl-4-thiazolyl)ethenyl)-8,8,12-trimethyl-4-aza-13,17-dioxabicyclo[14.1.0]heptadecane-5,9-dione

(4S,7R,8S,9S,13E,16S(E))-4,8-Dihydroxy-13-chloro-16-(1-methyl-2-(2-methyl-2-pyridyl)ethenyl)-1-aza-10-oxa-5,5,7,9-tetraamethyl-cyclohexadec-13-ene-2,6-dione

(1S,3S(E),7S,10R,11S,12S,16S)-16-Chloro-7,11-dihydroxy-3-(1-methyl-2-(2-methyl-2-pyridyl)ethenyl)-8,8,10,12-tetramethyl-4-aza-13,17-dioxabicyclo[14.1.0]heptadecane-5,9-dione

(1R,3S(E),7S,10R,11S,12S,16R)-16-Chloro-7,11-dihydroxy-3-(1-methyl-2-(2-methyl-2-pyridyl)ethenyl)-8,8,10,12-tetramethyl-4-aza-13,17-dioxabicyclo[14.1.0]heptadecane-5,9-dione

(4S,7R,8S,9S,13E,16S(Z))-4,8-Dihydroxy-13-chloro-16-(1-fluoro-2-(2-methyl-4-thiazolyl)ethenyl)-1-aza-10-oxa-5,5,7,9-tetraamethyl-cyclohexadec-13-ene-2,6-dione

(1S,3S(Z),7S,10R,11S,12S,16S)-16-Chloro-7,11-dihydroxy-3-(1-fluoro-2-(2-methyl-4-thiazolyl)ethenyl)-8,8,10,12-tetramethyl-4-aza-13,17-dioxabicyclo[14.1.0]heptadecane-5,9-dione

(1R,3S(Z),7S,10R,11S,12S,16R)-16-Chloro-7,11-dihydroxy-3-(1-fluoro-2-(2-methyl-4-thiazolyl)ethenyl)-8,8,10,12-tetramethyl-4-aza-13,17-dioxabicyclo[14.1.0]heptadecane-5,9-dione

(4S,7R,8S,9S,13E,16S(Z))-4,8-Dihydroxy-13-chloro-16-(1-chloro-2-(2-methyl-4-thiazolyl)ethenyl)-1-aza-10-oxa-5,5,7,9-tetraamethyl-cyclohexadec-13-ene-2,6-dione

(1S,3S(Z),7S,10R,11S,12S,16S)-16-Chloro-7,11-dihydroxy-3-(1-chloro-2-(2-methyl-4-thiazolyl)ethenyl)-8,8,10,12-tetramethyl-4-aza-13,17-dioxabicyclo[14.1.0]heptadecane-5,9-dione

(1R,3S(Z),7S,10R,11S,12S,16R)-16-Chloro-7,11-dihydroxy-3-(1-chloro-2-(2-methyl-4-thiazolyl)ethenyl)-8,8,10,12-tetramethyl-4-aza-13,17-dioxabicyclo[14.1.0]heptadecane-5,9-dione

(4S,7R,8S,9S,13E,16S(Z))-4,8-Dihydroxy-13-chloro-16-(1-fluoro-2-(2-methyl-4-thiazolyl)ethenyl)-5,5-trimethylene-1-aza-10-oxa-7,9-dimethyl-cyclohexadec-13-ene-2,6-dione

(1S,3S(Z),7S,10R,11S,12S,16S)-16-Chloro-7,11-dihydroxy-3-(1-fluoro-2-(2-methyl-4-thiazolyl)ethenyl)-8,8-trimethylene-10,12-dimethyl-4-aza-13,17-dioxabicyclo[14.1.0]heptadecane-5,9-dione

(1R,3S(Z),7S,10R,11S,12S,16R)-16-Chloro-7,11-dihydroxy-3-(1-fluoro-2-(2-methyl-4-thiazolyl)ethenyl)-8,8-trimethylene-10,12-dimethyl-4-aza-13,17-dioxabicyclo[14.1.0]heptadecane-5,9-dione

(4S,7R,8S,9S,13E,16S(Z))-4,8-Dihydroxy-13-chloro-16-(1-chloro-2-(2-methyl-4-thiazolyl)ethenyl)-5,5-trimethylene-1-aza-10-oxa-7,9-dimethyl-cyclohexadec-13-ene-2,6-dione

(1S,3S(Z),7S,10R,11S,12S,16S)-16-Chloro-7,11-dihydroxy-3-(1-chloro-2-(2-methyl-4-thiazolyl)ethenyl)-8,8-trimethylene-10,12-dimethyl-4-aza-13,17-dioxabicyclo[14.1.0]heptadecane-5,9-dione

(1R,3S(Z),7S,10R,11S,12S,16R)-16-Chloro-7,11-dihydroxy-3-(1-chloro-2-(2-methyl-4-thiazolyl)ethenyl)-8,8-trimethylene-10,12-dimethyl-4-aza-13,17-dioxabicyclo[14.1.0]heptadecane-5,9-dione

(4S,7R,8S,9S,13E,16S(Z))-4,8-Dihydroxy-13-chloro-16-(1-fluoro-2-(2-methyl-4-oxazolyl)ethenyl)-1-aza-10-oxa-5,5,7,9-tetraamethyl-cyclohexadec-13-ene-2,6-dione

(1S,3S(Z),7S,10R,11S,12S,16S)-16-Chloro-7,11-dihydroxy-3-(1-fluoro-2-(2-methyl-4-oxazolyl)ethenyl)-8,8,10,12-tetramethyl-4-aza-13,17-dioxabicyclo[14.1.0]heptadecane-5,9-dione

(1R,3S(Z),7S,10R,11S,12S,16R)-16-Chloro-7,11-dihydroxy-3-(1-fluoro-2-(2-methyl-4-oxazolyl)ethenyl)-8,8,10,12-tetramethyl-4-aza-13,17-dioxabicyclo[14.1.0]heptadecane-5,9-dione

(4S,7R,8S,9S,13E,16S(Z))-4,8-Dihydroxy-13-chloro-16-(1-fluoro-2-(2-methyl-2-pyridyl)ethenyl)-1-aza-10-oxa-5,5,7,9-tetraamethyl-cyclohexadec-13-ene-2,6-dione

(1S,3S(Z),7S,10R,11S,12S,16S)-16-Chloro-7,11-dihydroxy-3-(1-fluoro-2-(2-methyl-2-pyridyl)ethenyl)-8,8,10,12-tetramethyl-4-aza-13,17-dioxabicyclo[14.1.0]heptadecane-5,9-dione

(1R,3S(Z),7S,10R,11S,12S,16R)-16-Chloro-7,11-dihydroxy-3-(1-fluoro-2-(2-methyl-2-pyridyl)ethenyl)-8,8,10,12-tetramethyl-4-aza-13,17-dioxabicyclo[14.1.0]heptadecane-5,9-dione

(4S,7R,8S,9S,13E,16S(Z))-4,8-Dihydroxy-7-ethyl-13-chloro-16-(1-chloro-2-(2-methyl-2-pyridyl)ethenyl)-1-aza-10-oxa-5,5,9-trimethyl-cyclohexadec-13-ene-2,6-dione

(1S,3S(Z),7S,10R,11S,12S,16S)-16-Chloro-7,11-dihydroxy-7-ethyl-3-(1-chloro-2-(2-methyl-2-pyridyl)ethenyl)-8,8,12-trimethyl-4-aza-13,17-dioxabicyclo[14.1.0]heptadecane-5,9-dione

(1R,3S(Z),7S,10R,11S,12S,16R)-16-Chloro-7,11-dihydroxy-7-ethyl-3-(1-chloro-2-(2-methyl-2-pyridyl)ethenyl)-8,8,12-trimethyl-4-aza-13,17-dioxabicyclo[14.1.0]heptadecane-5,9-dione

(4S,7R,8S,9S,13E,16S)-4,8-Dihydroxy-13-fluoro-16-(2-methyl-5-benzothiazolyl)-1-aza-10-oxa-5,5,7,9-tetramethyl-cyclohexadec-13-ene-2,6-dione

(4S,7R,8S,9S,13Z,16S)-4,8-Dihydroxy-13-fluoro-16-(2-methyl-5-benzothiazolyl)-1-aza-10-oxa-5,5,7,9-tetramethyl-cyclohexadec-13-ene-2,6-dione

(1S,3S,7S,10R,11S,12S,16S)-16-Fluoro-7,11-dihydroxy-3-(2-methyl-5-benzothiazolyl)-8,8,10,12-tetramethyl-4-aza-13,17-dioxabicyclo[14.1.0]heptadecane-5,9-dione

(1R,3S,7S,10R,11S,12S,16R)-16-Fluoro-7,11-dihydroxy-3-(2-methyl-5-benzothiazolyl)-8,8,10,12-tetramethyl-4-aza-13,17-dioxabicyclo[14.1.0]heptadecane-5,9-dione

(4S,7R,8S,9S,13E,16S)-4,8-Dihydroxy-13-chloro-16-(2-methyl-5-benzothiazolyl)-1-aza-10-oxa-5,5,7,9-tetraamethyl-cyclohexadec-13-ene-2,6-dione

(4S,7R,8S,9S,13Z, 16S)-4,8-Dihydroxy-13-chloro-16-(2-methyl-5-benzothiazolyl)-1-aza-10-oxa-5,5,7,9-tetramethyl-cyclohexadec-13-ene-2,6-dione

(1S,3S,7S,10R,11S,12S,16S)-16-Chloro-7,1-dihydroxy-3-(2-methyl-5-benzothiazolyl)-8,8,10,12-tetramethyl-4-aza-13,17-dioxabicyclo[14.1.0]heptadecane-5,9-dione

(1R,3S,7S,10R,11S,12S,16R)-16-Chloro-7,11-dihydroxy-3-(2-methyl-5-benzothiazolyl)-8,8,10,12-tetramethyl-4-aza-13,17-dioxabicyclo[14.1.0]heptadecane-5,9-dione

(4S,7R,8S,9S,13E,16S)-4,8-Dihydroxy-7-ethyl-13-chloro-16-(2-methyl-5-benzothiazolyl)-1-aza-10-oxa-5,5,9-trimethyl-cyclohexadec-13-ene-2,6-dione

(1S,3S,7S,10R,11S,12S,16S)-16-Chloro-7,11-dihydroxy-10-ethyl-3-(2-methyl-5-benzothiazolyl)-8,8,12-trimethyl-4-aza-13,17-dioxabicyclo[14.1.0]heptadecane-5,9-dione

(1R,3S,7S,10R,11S,12S,16R)-16-Chloro-7,11-dihydroxy-10-ethyl-3-(2-methyl-5-benzothiazolyl)-8,8,12-tetramethyl-4-aza-13,17-dioxabicyclo[14.1.0]heptadecane-5,9-dione

(4S,7R,8S,9S,13E,16S)-4,8-Dihydroxy-7-allyl-13-chloro-16-(2-methyl-5-benzothiazolyl)-1-aza-10-oxa-5,5,9-trimethyl-cyclohexadec-13-ene-2,6-dione

(1S,3S,7S,10R,11S,12S,16S)-16-Chloro-7,11-dihydroxy-10-allyl-3-(2-methyl-5-benzothiazolyl)-8,8,12-trimethyl-4-aza-13,17-dioxabicyclo[14.1.0]heptadecane-5,9-dione

(1R,3S,7S,10R,11S,12S,16R)-16-Chloro-7,11-dihydroxy-10-allyl-3-(2-methyl-5-benzothiazolyl)-8,8,12-tetramethyl-4-aza-13,17-dioxabicyclo[14.1.0]heptadecane-5,9-dione

(4S,7R,8S,9S,13E,16S)-4,8-Dihydroxy-13-fluoro-16-(2-methyl-5-benzoxazolyl)-1-aza-10-oxa-5,5,7,9-tetramethyl-cyclohexadec-13-ene-2,6-dione

(4S,7R,8S,9S,13Z, 16S)-4,8-Dihydroxy-13-fluoro-16-(2-methyl-5-benzoxazolyl)-1-aza-10-oxa-5,5,7,9-tetramethyl-cyclohexadec-13-ene-2,6-dione

(1S,3S,7S,10R,11S,12S,16S)-16-Fluoro-7,11-dihydroxy-3-(2-methyl-5-benzoxazolyl)-8,8,10,12-tetramethyl-4-aza-13,17-dioxabicyclo[14.1.0]heptadecane-5,9-dione

(1R,3S,7S,10R,11S,12S,16R)-16-Fluoro-7,11-dihydroxy-3-(2-methyl-5-benzoxazolyl)-8,8,10,12-tetramethyl-4-aza-13,17-dioxabicyclo[14.1.0]heptadecane-5,9-dione

(4S,7R,8S,9S,13E,16S)-4,8-Dihydroxy-13-chloro-16-(2-methyl-5-benzoxazolyl)-1-aza-10-oxa-5,5,7,9-tetraamethyl-cyclohexadec-13-ene-2,6-dione

(4S,7R,8S,9S,13Z, 16S)-4,8-Dihydroxy-13-chloro-16-(2-methyl-5-benzoxazolyl)-1-aza-10-oxa-5,5,7,9-tetramethyl-cyclohexadec-13-ene-2,6-dione

(1S,3S,7S,10R,11S,12S,16S)— 16-Chloro-7,11-dihydroxy-3-(2-methyl-5-benzoxazolyl)-8,8,10,12-tetramethyl-4-aza-13,17-dioxabicyclo[14.1.0]heptadecane-5,9-dione

(1R,3S,7S,10R,11S,12S,16R)-16-Chloro-7,11-dihydroxy-3-(2-methyl-5-benzothiazolyl)-8,8,10,12-tetramethyl-4-aza-13,17-dioxabicyclo[14.1.0]heptadecane-5,9-dione

(1R,3S,7S,10R,11S,12S,16R)-16-Chloro-7,11-dihydroxy-3-(2-methyl-5-benzothiazolyl)-8,8,10,12-tetramethyl-4-aza-13,17-dioxabicyclo[14.1.0]heptadecane-5,9-dione

The production of the new epothilone derivatives, in which R⁵ is not halogen or cyano, is based on the linkage of three partial fragments A, B and C. The interfaces are as indicated in general formula I′.

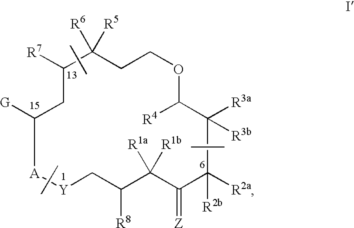

A means a C₁-C₆ fragment (epothilone numbering system) of general formula A-1 or A2

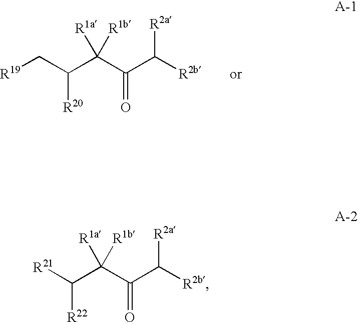

in which

R^{1a′}, R^{1b′}, R^{2a′} and R^{2b′} have the meanings already mentioned for R^{1a}, R^{1b}, R^{2a} and R^{2b}, and

R¹⁹ means CH₂OR^{19a}, CH₂-Hal, CHO, CO₂R^{19b}, or COHal,

R²⁰ means hydrogen, OR^{20a}, Hal, or OSO₂R^{20b},

R^{19a}, R^{20a} mean hydrogen, SO₂-alkyl, SO₂-aryl, SO₂-aralkyl or together a —(CH₂)ₒ group or together a CR^{23a}R^{23b} group,

R^{19b}R^{20b} mean hydrogen, C₁-C₂₀-alkyl, aryl, C₁-C₂₀-aralkyl,

R^{23a}, R^{23b} are the same or different and mean hydrogen, C₁-C₁₀-alkyl, aryl, C₇-C₂₀-aralkyl or together a —(CH₂)_{q} group,

0 means 2 to 4,

q means 3 to 6,

R²¹ means hydrogen,

R²² means hydroxyl, or

R²¹, R²² together mean an oxygen atom, or a C₂-C₁₀-alkylene-α,ω-dioxy group, which can be straight-chained or branched,

R²¹, R²² in each case mean a C₁-C₁₀-alkoxy group,

including all stereoisomers as well as their mixtures, and

free hydroxyl groups in R¹⁹, R²⁰ and R²² can be etherified or esterified, free carbonyl groups can be ketalized in A-I or A-2, converted into an enol ether or reduced, and free acid groups in A-1 or A-2 can be converted into their salts with bases.

B stands for a C₇-C₁₂ fragment (epothilone numbering system) of general formula

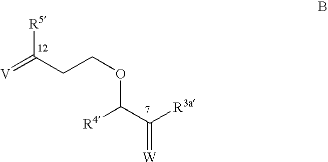

in which

R^{3′}, R^{4′}, and R^{5′} have the meanings already mentioned for R^{3a}, R⁴, and R⁵ (in addition R^{5′}=Hal, CN), and

V means an oxygen atom, two alkoxy groups OR²³, a C₂-C₁₀-alkylene-α,ω-dioxy group, which can be straight-chain or branched or H/OR²⁴,

W means an oxygen atom, two alkoxy groups OR²⁵, a C₂-C₁₀-alkylene-α,ω-dioxy group, which can be straight-chain or branched or H/OR²⁶,

R²⁴, R²⁶, independently of one another, mean hydrogen or a protective group PG²⁴,

R²³, R²⁵, independently of one another, mean C₁-C₂₀-alkyl.

C stands for a C13-C16 fragment (epothilone numbering system) of general formula

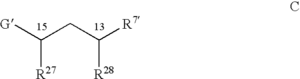

in which

G′ means a group

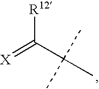

a bicyclic or tricyclic aryl radical,

R^{12′} has the meaning already mentioned in general formula I for R¹², and

R^{7′} means a hydrogen atom,

R²⁷ means halogen, N₃, NHR²⁹, a hydroxy group, a protected hydroxy group O-PG²⁷, a protected amino group NR²⁹PG²⁷, a C₁-C₁₀-alkylsulfonyloxy group, which optionally can be perfluorinated, a benzoyloxy group that is optionally substituted by C₁-C₄-alkyl, nitro, chlorine or bromine, an NR²⁹SO₂CH₃ group, an NR²⁹OC(═)CH₃ group, or a CH₂—C(═O)—CH₃ group,

R²⁸ means a hydroxy group, halogen, a protected hydroxy group OPG²⁸, a phosphonium halide radical PPh₃⁺ Hal⁻ (Ph=phenyl; Hal ═F, Cl, Br, I), a phosphonate radical P(O)(O)₂ (Q=C₁-C₁₀ alkyl or phenyl) or a phosphine oxide radical P(O)Ph₂ (Ph=phenyl),

X means an oxygen atom, two alkoxy groups OR^{13′}, a C₂-C₁₀-alkylene-α,ω-dioxy group, which can be straight-chain or branched, H/OR^{14′} or a grouping CR^{15′}R^{16′}, whereby

R^{13′} stands for a C₁-C₂₀-alkyl radical,

R^{14′} stands for hydrogen or a protective group PG¹⁴,

R^{15′}, R^{16′} are the same or different and stand for hydrogen, a C₁-C₂₀-alkyl aryl, or C₇-C₂₀-aralkyl radical.

The production of new epothilone derivatives, in which R is equal to halogen or cyano, is based on the linkage of two partial fragments A and D. The interfaces are as indicated in general formula I″.

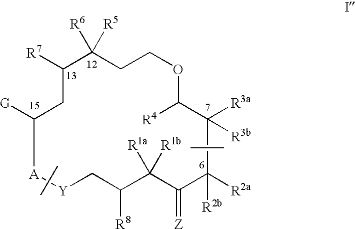

In this connection, A corresponds to the already-described C₁-C₆ fragment (epothilone numbering system) of general formula A-1 or A-2.

D stands for a C₇-C₁₅ fragment (epothilone numbering system) of general formula

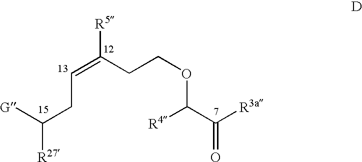

in which

R^{5″} stands for halogen or cyano, and R^{3a″}, R^{4″}, R^{27′} and G″ have the meanings already mentioned for R^{3a}, R⁴, R²⁷ and G.

As alkyl groups R^{1a}, R^{1b}, R^{2a}, R^{2b}, R^{3a}, R⁴, R⁵, R⁹, R¹², R¹³, R^{13′}, R¹⁵, R^{15′}, R¹⁶, R^{16′}, R^{19b}, R^{20b}, R²³ and R²⁵, straight-chain or branched-chain alkyl groups with 1-20 carbon atoms can be considered, such as, for example, methyl, ethyl, propyl, isopropyl, butyl, isobutyl, tert-butyl, pentyl, isopentyl, neopentyl, heptyl, hexyl, and decyl.

Alkyl groups R^{1a}, R^{1b}, R^{2a}, R^{2b}, R^{3a}, R⁴, R⁵, R⁹, R¹², R¹³, R^{13′}, R¹⁵, R^{15′}, R¹⁶R^{16′}, R^{19b}, R^{20b}, R²³, R^{23a}, R^{23b} and R²⁵ can be perfluorinated or substituted by 1-5 halogen atoms, hydroxy groups, C₁-C₄-alkoxy groups, or C₆-C₁₂-aryl groups (which can be substituted by 1-3 halogen atoms).

Asaryl radicals R^{1a}, R^{1b}, R^{2a}, R^{2b}, R^{3a}, R⁴, R⁵, R⁹, R¹², R¹⁵, R^{15′}, R¹⁶, R^{16′}, R^{19b}, R^{20b}, R^{23a}, and R^{23b}, substituted and unsubstituted carbocyclic or heterocyclic radicals with one or more heteroatoms, such as, e.g., phenyl, naphthyl, furyl, thienyl, pyridyl, pyrazolyl, pyrimidinyl, oxazolyl, pyridazinyl, pyrazinyl, quinolyl, thiazolyl, benzothiazolyl, or benzoxazolyl, which can be substituted in one or more places by halogen, OH, O-alkyl, CO₂H, CO₂-alkyl, —NH₂, —NO₂, —N₃, —CN, C₁-C₂₀-alkyl, C₁-C₂₀-acyl, or C₁-C₂₀-acyloxy groups, are suitable.

As bicyclic and tricyclic aryl radicals G, substituted and unsubstituted carbocyclic or heterocyclic radicals with one or more heteroatoms, such as, e.g., naphthyl, anthryl, benzothiazolyl, benzoxazolyl, benzimidazolyl, quinolyl, isoquinolyl, benzoxazinyl, benzofuran, indolyl, indazolyl, quinoxalinyl, tetrahydroisoquinolinyl, tetrahydroquinolinyl, thienopyridinyl, pyridopyridinyl, benzopyrazolyl, benzotriazolyl, dihydroindolyl, which can be substituted in one or more places by halogen, OH, O-alkyl, CO₂H, CO₂-alkyl, —NH2, —NO₂, —N₃, —CN, C₁-C₂₀-alkyl, C₁-C₂₀-acyl, or C₁-C₂₀-acyloxy groups, are suitable.

The aralkyl groups in R^{1a}, R^{1b}, R^{2a}, R^{2b}, R^{3a}, R⁴, R⁵, R⁹, R¹², R¹⁵, R^{15′}, R¹⁶, R^{16′}, R^{19b}, R^{20b}, R^{23a} and R^{23b} can contain in the ring up to 14 C atoms, preferably 6 to 10 C atoms, and in the alkyl chain 1 to 8 atoms, preferably 1 to 4 atoms. As aralkyl radicals, for example, benzyl, phenylethyl, naphthylmethyl, naphthylethyl, furylmethyl, thienylethyl, and pyridylpropyl are considered. The rings can be substituted in one or more places by halogen, OH, O-alkyl, CO₂H, CO₂-alkyl, —NO₂, —N₃, —CN, C₁-C₂₀-alkyl, C₁-C₂₀-acyl, or C₁-C₂₀-acyloxy groups.

The alkoxy groups that are contained in R²¹, R²² and X in general formula I are in each case to contain 1 to 20 carbon atoms, whereby methoxy, ethoxy, propoxy, isopropoxy and t-butyloxy groups are preferred.

As representatives of protective groups PG, alkyl- and/or aryl-substituted silyl, C₁-C₂₀-alkyl, C₄-C₇-cycloalkyl, which in addition in the ring can contain an oxygen atom, aryl, C₇-C₂₀-aralkyl, C₁-C₂₀-acyl as well as aroyl can be mentioned.

As alkyl, silyl and acyl radicals for protective groups PG, the radicals that are known to one skilled in the art are considered. Preferred are alkyl or silyl radicals that can be easily cleaved from the corresponding alkyl and silyl ethers, such as, for example, methoxymethyl, methoxyethyl, ethoxyethyl, tetrahydropyranyl, tetrahydrofuranyl, trimethylsilyl, triethylsilyl, tert-butyldimethylsilyl, tert-butyldiphenylsilyl, tribenzylsilyl, triisopropylsilyl, benzyl, para-nitrobenzyl, para-methoxybenzyl radicals as well as alkylsulfonyl and arylsulfonyl radicals. As acyl radicals, e.g., formyl, acetyl, propionyl, isopropionyl, pivalyl, butyryl or benzoyl, which can be substituted with amino and/or hydroxy groups, are suitable.

As amino protective groups, the radicals that are known to one skilled in the art can be considered. For example, the alloc, boc, Z, benzyl, f-moc, troc, stabase or benzostabase groups can be mentioned.

Acyl groups PG can contain 1 to 20 carbon atoms, whereby formyl, acetyl, propionyl, isopropionyl and pivalyl groups are preferred.

Index m in the alkylene group that is formed from R^{1a} and R^{1b} preferably stands for 1, 2, 3 or 4. 2122

The C₂-C₁₀-alkylene-α,ω-dioxy group that is possible for R²¹, R²², V, W and X is preferably an ethyleneketal or neopentylketal group.

Representation of Partial Fragments A:

The partial fragments (synthesis components) of general formulas A-1 and A2 can be produced as in DE 197 51200.3, DE 199 07 480.1, DE 199 21 0861.1 and WO 99/07692.

Representation of Partial Fragments B:

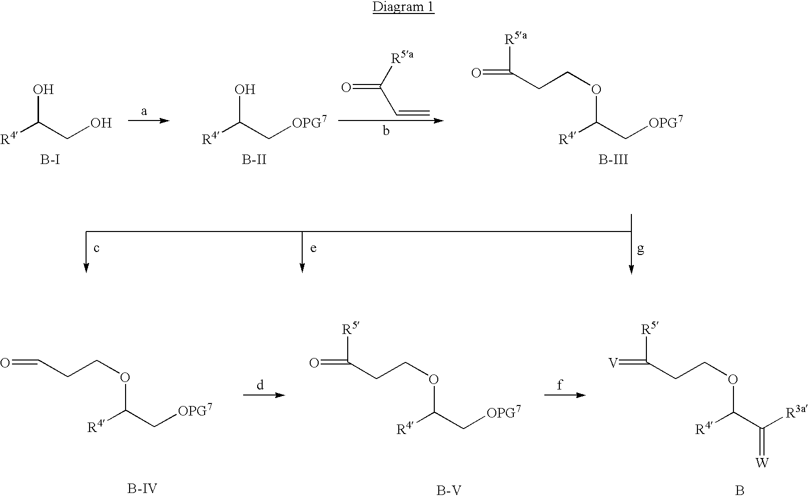

The representation of fragments of type B, in which R^{3a′}, R^{4′}, R^{5′}, V and W can have all the meanings already mentioned, is shown in Diagram 1. The synthesis can be performed starting from both enantiomer-pure compounds B-I and racemic compounds. Step a (B-I [symbol] B-11):

Compounds of type B-I are in some cases commercially available. In these cases, the primary alcohol function is selectively protected according to the process that is known to one skilled in the art. In principle, all protective groups that are mentioned, e.g., for PG⁵ are suitable. Especially preferred, is, e.g., the tetrahydropyranyl protective group. For compounds of B-1 type that are not commercially available, radical R^{4′} is produced according to the processes known to one skilled in the art, e.g., by nucleophilic substitution on corresponding aldehydes.

Step b (B-II [symbol] B-III):

By 1,4-addition of compounds of type B-II to α,β-unsaturated carbonyl compounds, compounds of type B-III are obtained. In this connection, R^{5′a} can have all meanings already mentioned for R⁵ and in addition can be equal to O-alkyl.

Step c (B-III [symbol] B-IV):

For compounds of type B-III, in which R^{5′a} has the meaning of O-alkyl, B-IV can be reduced to the aldehyde. The conversion into the aldehyde is carried out either directly, e.g., by reduction with diisobutylaluminum hydride at low temperatures (below −40° C.) or else in two stages by reduction to alcohol and subsequent oxidation. In this respect, the processes known to one skilled in the art are used. For the reduction, e.g., complex hydrides such as lithium aluminum hydride are used. Oxidation can be carried out, e.g., according to the processes that are described for the production of A-III.

Step d (B-IV [symbol] B-V):

By nucleophilic addition of organometallic compounds of theoretical formula M-R^{5′}, in which M stands for indium, an alkali metal, preferably lithium, or a divalent metal MX, in which X represents a halogen and radical R^{5″} has the above-mentioned meanings. As a divalent metal, magnesium and zinc are preferred; as halogen X, chlorine, bromine and iodine are preferred.

Step e (B-III [symbol] B-IV)

For compounds in which R^{5′a} stands for O-alkyl, compounds of type B-V can also be directly obtained by nucleophilic addition. For this purpose, methods that are known to one skilled in the art are used, such as, e.g., the use of dialkyl copper lithium compounds.

Step f (B-V [symbol] B):

The conversion of B-V into partial fragments of general formula B is carried out analogously to the processes that are described in WO 99/07692. Step g (B-III [symbol] B):

For compounds in which R^{5′a} does not mean O-alkyl, compounds B-E also can be converted into partial fragments of type B analogously to WO 99/07692.

Representation of Partial Fragments C:

The partial fragments (synthesis components) of general formula C can be produced as in DE 197 51200.3, DE 199 07 480.1 and WO 99/07692.

Representation of partial fragments ABC and their cyclization to I is also carried out analogously to how it is described for numerous epothilone derivatives in WO 99/07692.

WO 99/07692 already confirms the general applicability of the synthesis principle that is described below for the compounds according to the invention. In addition, numerous synthesis components of general formulas A, B and C, with which others of the compounds of general formula I claimed here can be obtained, stem from WO 99/07692. Synthesis components of general formula C, in which a halogen atom, especially a fluorine, chlorine or bromine atom, is present as R¹², are subjects of DE 199 07 480.1 and PCT/EP00/01333.

Representation of Partial Fragments D:

The synthesis of partial fragments D is described in Diagram 2 below starting from the aldehydes of general formula D-I.

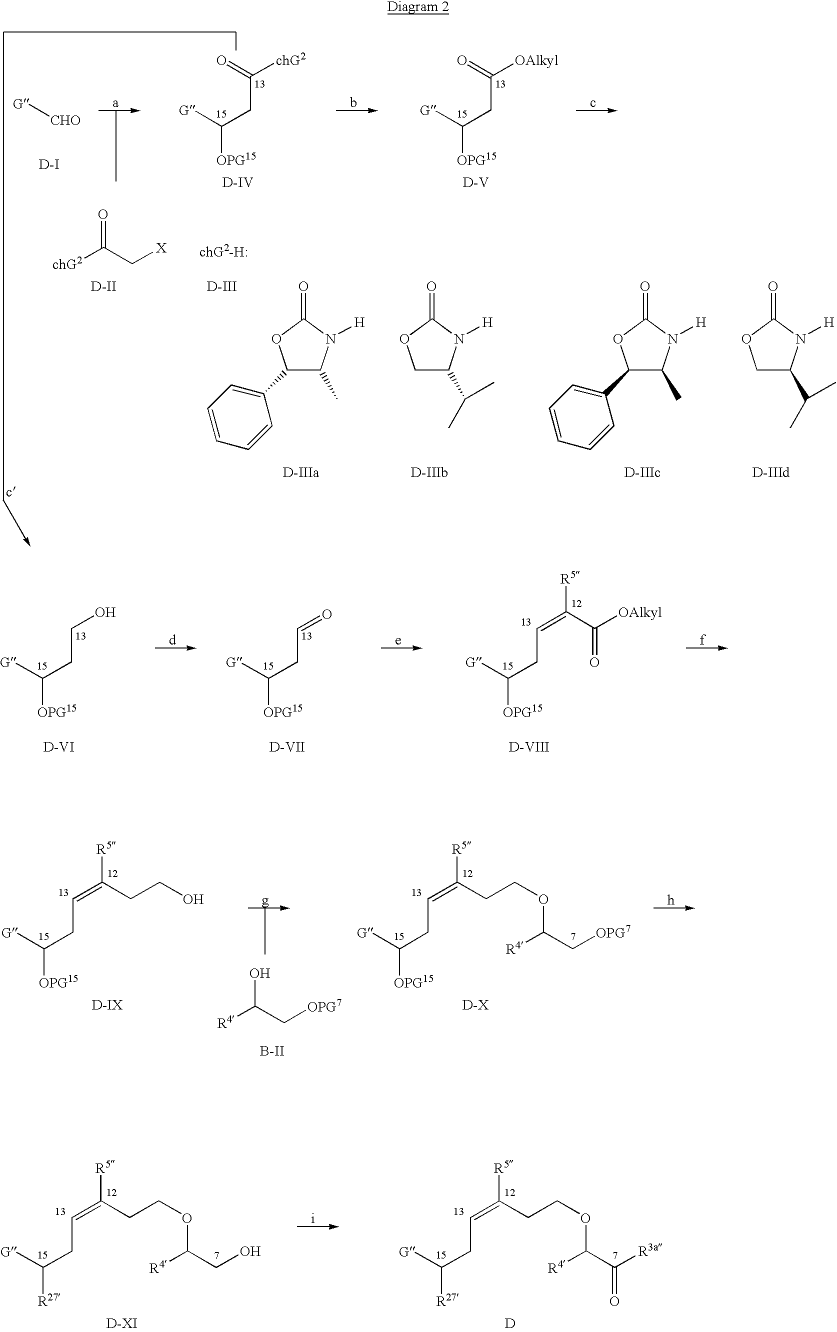

Step a (D-I→D-IV):

Compound D-I is alkylated with the enolate of a carbonyl compound of general formula D-II, in which X can be a hydrogen, and chG² can be a chiral auxiliary group according to the methods that are known to one skilled in the art. The enolate is produced by action of strong bases, such as, e.g., lithium diisopropylamide or lithium hexamethyldisilazane at low temperatures. Another possibility lies in a kind of Reformatzsky reaction in which the compound of general formula D-II with X halogen, preferably chlorine or bromine, is converted with CrCl₂ into an organometallic reagent, which then is reacted with aldehyde D-I to form D-IV. As chiral auxiliary group chG²-H (DIII), chiral alcohols that can be produced in an optically pure and inexpensive manner, such as, e.g., pulegol, 2-phenylcyclohexanol, 2-hydroxy-1,2,2-triphenylethanol, 8-phenylmenthol or compounds that contain reactive NH-groups that can be produced in an optically pure and inexpensive manner, such as, e.g., amines, amino acids, lactams or oxazolidinones, are suitable. Preferred are oxazolidinones; especially preferred are the compounds of formulas D-IIIa to D-IIId. The absolute stereochemistry on the α-carbonylcarbon of the compound of general formula D-IV is set by the selection of the respective antipode. In this way, the compounds of general formulas D-IV to D-XV or their respective enantiomers ent-D-IV to ent-D-XV can be obtained in an enantiomer-pure manner. If an achiral alcohol, such as, e.g., ethanol, is used as chG²-H (D-III), the racemic compounds rac-D-IV to rac-D-XV are obtained.

The free hydroxyl group in B-IV is then protected according to the methods that are known to one skilled in the art. As protective groups PG15, the protective groups that are known to one skilled in the art, as were already mentioned above for PG5 (A-I→A-II), are suitable.

Preferred are silicon-containing protective groups, which can be cleaved under acidic reaction conditions or with use of fluoride, such as, e.g., trimethylsilyl, triethylsilyl, tert-butyldimethylsilyl, tert-butyldiphenylsilyl, tribenzylsilyl and triisopropylsilyl radicals.

Especially preferred are the tert-butyldiphenylsilyl radical and the tert-butyldimethylsilyl radicals.

Step b (D-IV→D-V):

If group chG² represents one of the chiral auxiliary groups that are mentioned under step a, the latter is recovered by reesterification of D-IV in an alkyl ester of general formula D-V. The reesterification is carried out according to the methods that are known to one skilled in the art. Preferred is reesterification with simple alcohols, such as, e.g., methanol or ethanol in the presence of corresponding titanium(IV) alcoholates.

Step c (D-V→D-VI):

The ester in D-V is reduced to alcohol D-VI. As a reducing agent, the reducing agents that are known to one skilled in the art, such as, e.g., aluminum hydrides, such as, e.g., lithium aluminum hydride or diisobutylaluminum hydride, are suitable. The reaction is carried out in an inert solvent, such as, e.g., diethyl ether, tetrahydrofuran, or toluene.

Step c′ (D-IV→D-VI):

As an alternative to steps b) and c), the carbonyl group in D-IV can be reduced directly to the alcohols of general formula D-VI according to the conditions that are mentioned under step c). Here, chiral auxiliary component chG²-H can also be recovered.

Step d (D-VI→D-VII):

The oxidation of the primary alcohol in D-VI to the aldehyde of general formula D-VII is carried out according to the processes that are known to one skilled in the art. For example, oxidation with pyridinium chlorochromate, pyridinium dichromate, the chromium trioxide-pyridine complex, oxidation according to Swem or related methods, e.g., with use of the SO₃-pyridine complex or oxalyl chloride in dimethyl sulfoxide, the use of Dess-Martin periodinane, and the use of nitrogen oxides, such as, e.g., N-methyl-morpholino-N-oxide in the presence of suitable catalysts, such as, e.g., tetrapropylammonium perruthenate in inert solvents, can be mentioned. Preferred is the oxidation according to Swern, the SO₃-pyridine complex as well as with N-methyl-morpholino-N-oxide using tetrapropylammonium perruthenate.

Step e (D-VII→D-VIII):

The unsaturated esters of general formula D-VIII are produced by the processes that are known to one skilled in the art. In this respect, methods such as, e.g., the Wittig reaction or the Wittig/Horner reaction, or else the Peterson olefination are suitable. Preferred is the Wittig/Horner reaction with use of phosphonates of type alkylOOC—CHR^{5″}-P(O)(Oalkyl′)₂, whereby alkyl and alkyl′ can be the same or different and preferably mean methyl, ethyl, i-propyl, or trifluoroethyl, and R5′ has the already mentioned meaning, with bases such as, e.g., potassium carbonate, sodium hydride, n-butyllithium, potassium-tert-butanolate, sodium ethanolate, lithium hexamethyldisilazane, sodium hexamethyldisilazane, potassium hexamethyldisilazane and optionally with the additions of, for example, crown ethers, DMPU or HMPA, in solvents such as methanol, tetrahydrofuran, dimethylformamide, or diethyl ether; the combination of potassium carbonate in methanol, sodium hydride in dimethylformamide or tetrahydrofuran and potassium hexamethyldisilazane with 18-crown-6 in tetrahydrofuran is preferred.

The E/Z diastereomers that are obtained can be separated, for example, in this step or in the next step, and can be converted individually per se into the corresponding E- or Z-olefin end products. In the formula diagram, only the E-form is shown for the sake of clarity. All of the following steps also hold true, however, for the corresponding Z-isomer.

Step f (D-VIII→D-IX):

Compounds of type D-VIII are converted by C₁-extension into compounds of type D-IX. This C₁-extension is carried out according to multistage processes. For example, the ester group in D-VIII can be reduced to a primary alcohol. As a reducing agent, the reducing agents that are known to one skilled in the art, such as, e.g., aluminum hydrides, such as, e.g., lithium aluminum hydride or diisobutylaluminum hydride, are suitable. The reaction is carried out in an inert solvent, such as, e.g., diethyl ether, tetrahydrofuran, or toluene. The primary alcohol can then be converted into a leaving group, such as, e.g., a halide or an OSO₂-alkyl, O—SO₂-aryl or OSO₂-aralkyl group. The introduction of the later C-14 can then be carried out by, e.g., substitution by cyanide with use of NaCN or KCN. The nitrile that is formed is then converted by reduction with, e.g., diisobutylaluminum hydride and acid cleavage of the primary formed imine into an aldehyde, which then is converted with, e.g., lithium aluminum hydride, sodium borohydride or diisobutylaluminum hydride in the primary alcohol of type D-IX.

Step g (D-IX+B-II→D-X):

The production of compounds of type D-X is then carried out by linkage of D-IX with compounds of type B-II that were already described. The latter can be performed, e.g., with use of triphenylphosphine and azodiesters, such as, for example, azodicarboxylic acid diethyl ester. As an alternative to the above, one of the two hydroxy groups (in component D-IX or B-II) can also be converted into a halide or an OSO₂alkyl, OSO₂aryl or OSO2aralkyl group. The leaving group is preferably formed on the primary alcohol function in component D-IX. To link both components, the free hydroxyl group is then deprotonated in the respective other components, preferably B-II, with suitable bases, such as, for example, sodium hydride, n-butyllithium, 4-dimethylamino-pyridine, Hünig base, alkylihexamethyldisilazanes and by nucleophilic substitution in compounds of type D-X.

Step h (D-X→D-XI):

If R^{27′}=OPG²⁷, protective group PG⁷ is now cleaved according to the process that is known to one skilled in the art. If this is a protective group that can be cleaved acidically, then cleavage can be accomplished with dilute mineral acids in aqueous-alcoholic solutions and with the use of catalytic quantities of acids, such as, e.g., para-toluenesulfonic acid, para-toluenesulfonic acid-pyridinium salt, or camphorsulfonic acid in alcoholic solutions, preferably in ethanol or isopropanol.

If A is to be an NR¹⁷ group in the compounds of formula I, first protective group PG²⁷ is cleaved selectively before the cleavage of protective group PG⁷ according to the methods that are known to one skilled in the art (also see above in this respect). The thus obtained secondary alcohol is converted with a sulfonyl chloride or a sulfonic acid anhydride into a sulfonate and optionally then in a Finkelstein reaction with an alkali bromide or alkali chloride, or by reaction of the secondary alcohol with CBr4 in the presence of triphenylphosphine or bis(diphenylphosphinoethane) into a secondary halide. The thus obtained halides or sulfonates can then be converted into a corresponding azide (L′=N₃) by a nucleophilic substitution with, e.g., sodium azide in a neutral polar solvent, such as, for example, dimethylformamide or dimethyl sulfoxide. The above-described cleavage of protective group PG⁷ would then follow.

Step i (D-XI→D):

The oxidation of the primary alcohol in D-XI to the corresponding aldehyde is carried out according to the processes that are known to one skilled in the art. For example, oxidation with pyridinium chlorochromate, pyridinium dichromate, the chromium trioxide-pyridine complex, oxidation according to Swem or related methods, e.g., with use of the SO₃-pyridine complex or oxalyl chloride in dimethyl sulfoxide, the use of Dess-Martin periodinane, or the use of nitrogen oxides, such as, e.g., N-methyl-morpholino-N-oxide in the presence of suitable catalysts, such as, e.g., tetrapropylammonium perruthenate in inert solvents, can be mentioned. Preferred is the oxidation according to Swern, as well as with N-methyl-morpholino-N-oxide using tetrapropylammonium perruthenate.

If R^{3a″}≠H, the corresponding secondary alcohol can now be produced according to the methods that are known to one skilled in the art with organometallic compounds of general formula M-R^{3a″}, in which M stands for an alkali metal, preferably lithium, or a divalent metal MX, in which X represents a halogen, and radical R^{3a″} has the above-mentioned meaning. As a divalent metal, magnesium and zinc are preferred; as halogen X, chlorine, bromine and iodine are preferred.

The thus obtained secondary alcohol is converted by oxidation into the ketone of general formula D with R3′≠H according to the process initially mentioned under i). The oxidation with N-methyl-morpholino-N-oxide with use of tetrapropylammonium perruthenate is preferred.

Partial Fragments of General Formula AB

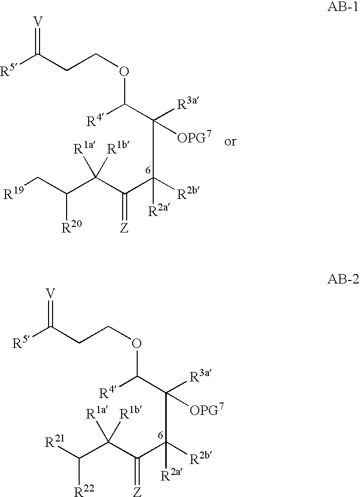

in which R^{1a′}, R^{1b′}, R^{2a′}, R^{2b′}, R^{3a′}, R^{4′}, R^{5′}, R¹⁹, R²⁰, R²¹, R²², V and Z have the meanings already mentioned, and PG⁷ represents a hydrogen atom or a protective group PG, are obtained from previously mentioned fragments A and B according to the process that is shown in Diagram 3.

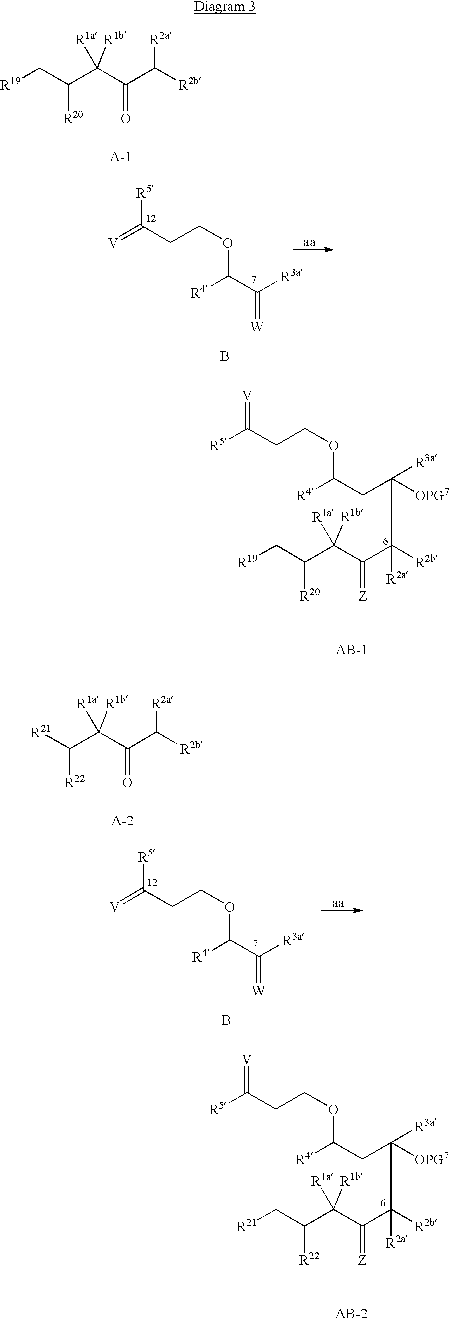

Step aa (A+B [symbol] AB):

Compound B, in which W has the meaning of an oxygen atom and optionally present additional carbonyl groups are protected, is alkylated with the enolate of a carbonyl compound of general formula A. The enolate is produced by action of strong bases, such as, e.g., lithium diisopropylamide, or lithium hexamethyldisilazane, at low temperatures.

Partial Fragments of General Formula BC

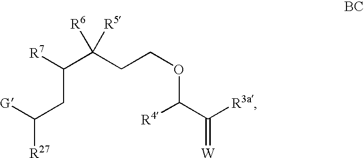

in which R^{3a}′, R^{4′}, R^{5′}, R⁶, R⁷, R²⁷, G and W have the already mentioned meanings, are obtained from the previously described fragments B and C according to the process that is shown in Diagram 4.

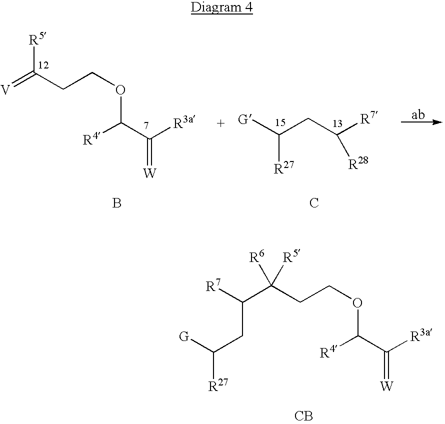

Step ab(B+C [symbol] BC):

Compound C, in which R²⁸ has the meaning of a Wittig salt, and optionally present additional carbonyl groups are protected, is deprotonated by a suitable base, such as, e.g., n-butyllithium, lithium diisopropyl amide, potassium tert-butanolate, sodium or lithium-hexamethyldisilazide and reacted with a compound B, in which V has the meaning of oxygen, and W has the meaning of two alkoxy groups OR²⁵, a C₂-C₁₀-alkylene-α,ω-dioxy group, which can be straight-chain or branched or has H/OR²⁶.

Partial Fragments of General Formula ABC (AB+C)

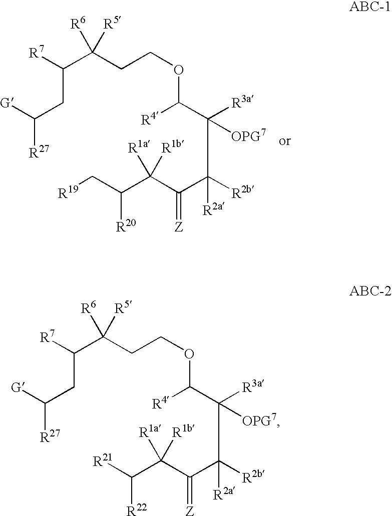

in which R^{1a′}, R^{1b′}, R^{2a′}, R^{2b′}, R^{3a′}, R^{4′}, R^{5′}, R⁶, R⁷, R¹⁹, R²⁰, R²¹ R²², G′ and Z have the meanings already mentioned, and PG⁷ represents a hydrogen atom or a protective group PG, are obtained from the previously described fragments AB and C according to the process shown in Diagram 5 and Diagram 6.

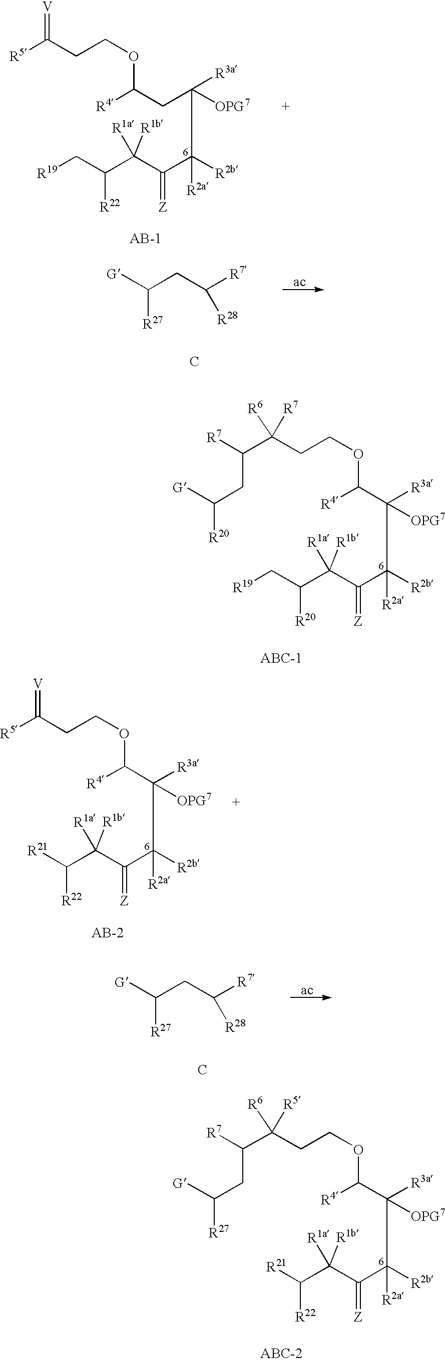

Step ac (AB+C [symbol] ABC):

Compound C, in which R²⁸ has the meaning of a Wittig salt, and optionally present additional carbonyl groups are protected, is deprotonated by a suitable base, such as, e.g., n-butyllithium, lithium diisopropyl amide, potassium tert-butanolate, sodium or lithium-hexamethyldisilazide and reacted with a compound AB, in which V has the meaning of an oxygen atom.

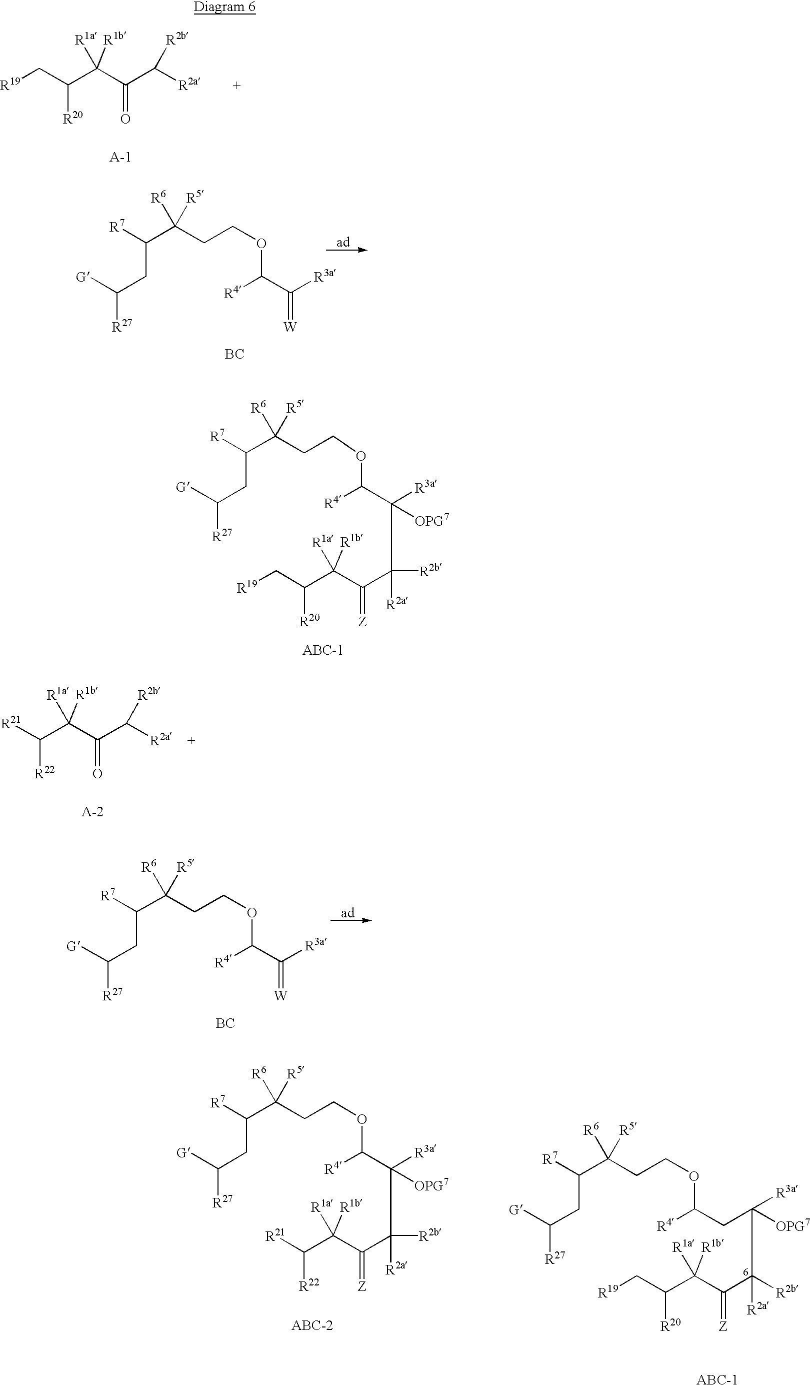

Step ad (A+BC [symbol] ABC):

Compound BC, in which W has the meaning of an oxygen atom and optionally present additional carbonyl groups are protected, is alkylated with the enolate of a carbonyl compound of general formula A. The enolate is produced by action of strong bases, such as, e.g., lithium diisopropylamide, or lithium hexamethyldisilazane, at low temperatures.

Representation of Partial Fragments AD:

Partial Fragments of General Formula AD

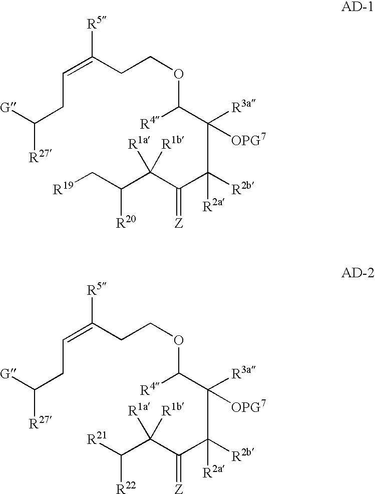

in which R^{1a′}, R^{1b′}, R^{2a′}, R^{2b′}, R^{3a}′, R^{4′}, R^{5″}, R¹⁹, R²⁰, R^{27′}, G″ and Z have the already mentioned meanings, are obtained from the previously described fragments A and D according to the process shown in Diagram 7.

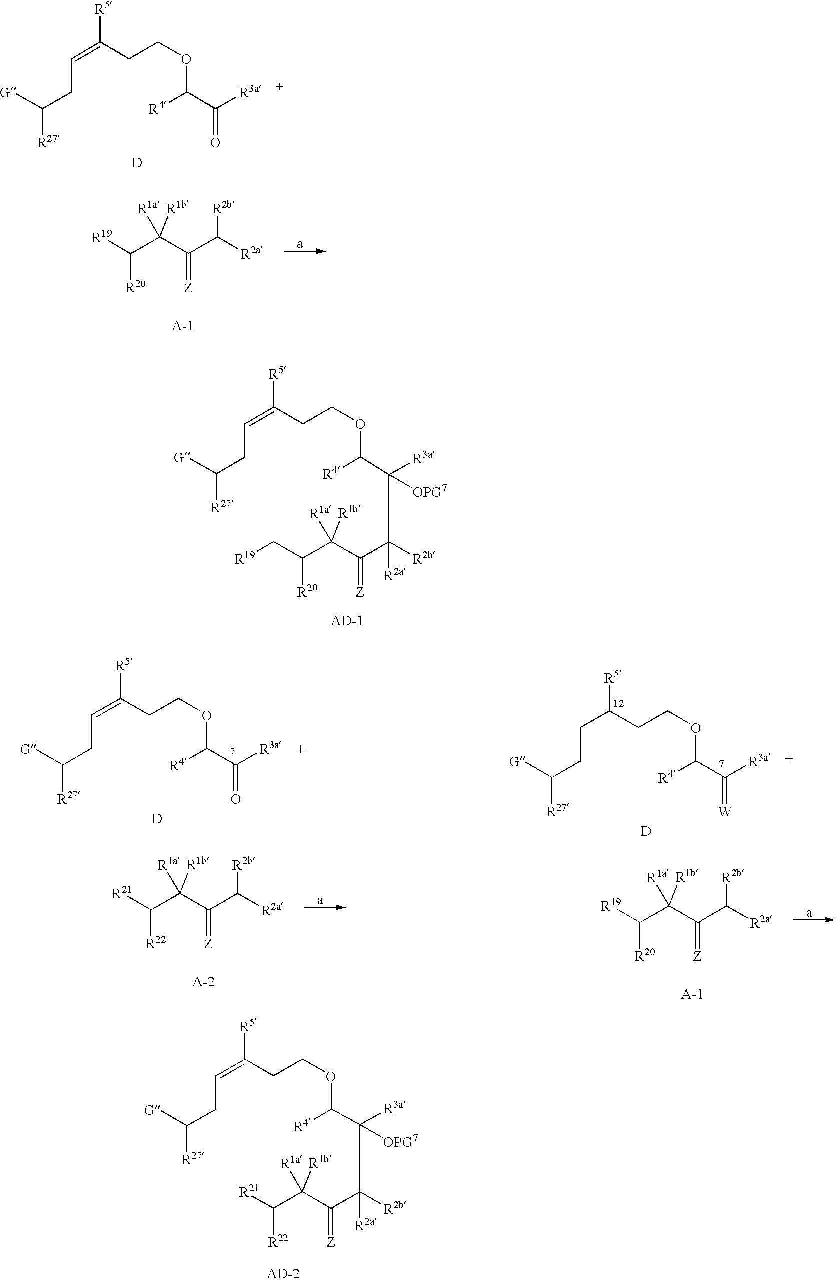

Step a (A+D [symbol] AD):

Compound D is alkylated with the enolate of a carbonyl compound of general formula A. The enolate is produced by action of strong bases, such as, e.g., lithium diisopropylamide, or lithium hexamethyldisilazane, at low temperatures.

The conversion of fragments ABC or AD in compounds of general formula I is carried out according to the processes that are described below. The one difference between fragments ABC and AD consists in that in fragments ABC, radical R^{5′} can have all meanings of R⁵ except halogen and cyano, while in fragments AD, R^{5″} means halogen or cyano.

Step ae (ABC-1 or AD-1 [symbol] I):

Compounds ABC-I or AD-I, in which R¹⁹ represents a carboxylic acid CO₂H and R²⁷ represents a hydroxyl group or an amino group, are reacted according to the methods known to one skilled in the art for forming large macrolides or macrolactams to form compounds of formula I, in which A-Y has the meaning of an O—(C═O) group or NR²⁹—C(C═O) group. For example, the method, described in “Reagents for Organic Synthesis, Vol. 16, p. 353,” with use of 2,4,6-trichlorobenzoic acid chloride and suitable bases, such as, e.g., triethylamine, 4-dimethylaminopyridine, and sodium hydride, is preferred for lactone formation. For example, the reaction of amino acid (R¹⁹ a carboxylic acid CO₂H and R²⁷ an NHR²⁹ group) with diphenylphosphorylazide in the presence of a base is preferred for the lactam formation.

Step af (ABC-1 or AD-1 [symbol] I):

Compounds ABC-1 or AD-1, in which R¹⁹ represents a group CH₂OH and R²⁷ represents a hydroxyl group, can be reacted preferably with use of triphenylphosphine and azodiesters, such as, for example, azodicarboxylic acid diethyl ester, to form compounds of formula I, in which A-Y has the meaning of an O—CH₂ group.

Compounds ABC or AD, in which R¹⁹ represents a group CH₂-Hal or CH₂OSO₂alkyl or CH₂OSO₂aryl or CH₂OSO₂aralkyl, and R²⁷ represents a hydroxyl group, can be cyclized after deprotonation with suitable bases, such as, for example, sodium hydride, n-butyllithium, 4-dimethylaminopyridine, Hunig base, or alkylihexamethyldisilazanes, to form compounds of formula I, in which A-Y has the meaning of an O—CH₂ group.

Step ag (ABC-2 or AD-2 [symbol] I)

Compounds ABC-2 or AB-2, in which R²¹ and R22 and together represent an oxygen atom R²⁷ represents an NR²⁹SO₂CH₃ group, can be cyclized by the action of strong bases, such as, e.g. lithium diisopropylamide, or lithium hexamethyldisilazane at low temperatures to form sulfonamide I, in which A-Y has the meaning of an NR²⁹SO₂ group.

Step ah (ABC-2 or AD-2 [symbol] I):

Compounds ABC-2 or AD-2, in which R²¹ and R²² together represent an oxygen atom and R²⁷ represents O—C(═)CH₃ group, can be cyclized by action of strong bases, such as, e.g., lithium diisopropylamide, or alkali hexamethyldisilazane at low temperatures to form lactone I, in which A-Y has the meaning of an O—C(═O) group.

Step ai (ABC-2 or AD-2 [symbol] I):

Compounds ABC-2 or AD-2, in which R²¹ and R²²¹ together represent an oxygen atom and R²⁷ represents a CH₂C(═O)CH₃ group, can be cyclized by action of strong bases, such as, e.g., lithium diisopropylamide, or alkali hemamethyldisilazane at low temperatures to form lactone I, in which A-Y has the meaning of a CH₂C(═O) group.

Introduction of the Nitrogen Group for R²⁷:

Amino group NHR²⁹ can be introduced in the step of the C fragment, the BC fragment or the ABC fragment according to the methods that are known to one skilled in the art. The production from the azide (R²⁷=N₃), which is converted according to the methods known to one skilled in the art, preferably with use of a phosphine, such as, for example, triphenylphosphine in the presence of water, into the amine (R²⁷=NHR²⁹) that is optionally to be protected for the further course of the reaction, is preferred. The introduction of the azide can be carried out with use of the Mitsunobu reaction in the presence of metal azides, preferably sodium or zinc azide, or by substitution of a suitable leaving group, such as, for example, a chlorine, bromine, or iodine atom, an alkylsulfonyloxy group, a perfluorinated alkylsulfonyloxy group, an arylsulfonyloxy group or an aralkylsulfonyloxy group by azides.

The flexible functionalization of described components A, B, and C also ensures a linkage sequence that deviates from the above-described process and that leads to components ABC. These processes are listed in the following table:

| Possible Linkages | Linkage Methods a to e | Prerequisites |
|---|---|---|
| A + B [symbol] A − B | a: Aldol (see Diagram 3) | Z = W = oxygen |
| B + C [symbol] B − C | b: Wittig (analogously to | U = oxygen and R²⁸ = |
| | Diagram 4) | Wittig salt, phosphine oxide |
| | | or phosphonate |
| | e: McMurry | U = V = oxygen |
| A + C [symbol] A − C | c: Esterification (e.g., | R¹⁹ = CO₂R^{19b} or COHal |
| | 2,4,6-trichlorobenzoyl | and R²⁷ = hydroxyl |
| | chloride and 4-dimethyl- | |
| | amino-pyridine) | |
| | d: Etherification (e.g., | R¹⁹ = CH₂OH and R²⁷ = |
| | according to Mitsunobu) | hydroxyl or OSO₂-alkyl or |
| | | OSO₂-aryl or OSO₂-aralkyl |
| | f. Amide formation (e.g., | R¹⁹ = CO₂R^{19b} or COHal |
| | with (PhO)₂P(O)N₃) | and R²⁷ = NHR²⁹ |
| | in the presence of a base | |
| | in an inert solvent. | |
| | g: Ketone formation by | R²⁷ = CH₂C(═O)CH₃ and |
| | aldol reaction with a | R²¹, R²² = oxygen |
| | strong base. | |
| | h. Sulonamide formation | R²⁷ = NR²⁹SO₂CH₃ and |
| | in the presence of a | R²¹, R²² = oxygen |
| | strong base. | |
| | i: Amide formation in the | R²⁷ = NR²⁹ C(═O)CH₃ and |
| | presence of a strong base | R²¹, R²² = oxygen |

According to these processes, components A, B and C, as indicated in Diagram 10, can be linked:

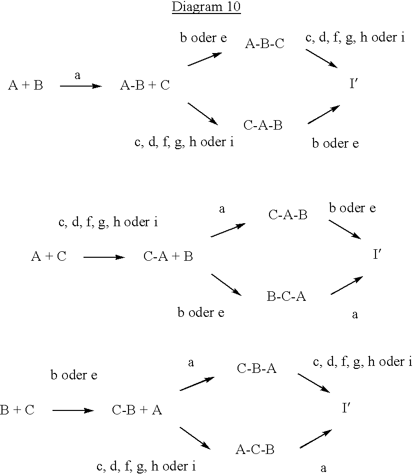

[Key: oder=or]

Free hydroxyl groups in I, A, B, C, AB, BC and ABC can be further functionally modified by etherification or esterification, free carbonyl groups by ketalization, enol ether formation or reduction.

The invention relates to all stereoisomers of these compounds and also their mixtures.

In addition, the invention relates to all prodrug formulations of these compounds, i.e., all compounds that release in vivo a bioactive active ingredient component of general formula I.

Biological Actions and Applications of the New Derivatives:

The new compounds of formula I are valuable pharmaceutical agents. They interact with tubulin by stabilizing microtubuli that are formed and are thus able to influence the cell-splitting in a phase-specific manner. This relates mainly to quick-growing, neoplastic cells, whose growth is largely unaffected by intercellular regulating mechanisms. Active ingredients of this type are in principle suitable for treating malignant tumors. As applications, there can be mentioned, for example, the treatment of ovarian, stomach, colon, adeno-, breast, lung, head and neck carcinomas, malignant melanoma, acute lymphocytic and myelocytic leukemia. The compounds according to the invention are suitable owing to their properties basically for anti-angiogenesis therapy as well as for treatment of chronic inflammatory diseases, such as, for example, psoriasis, multiple sclerosis, or arthritis. To avoid uncontrolled proliferation of cells and for better compatibility of medical implants, they can basically be applied or introduced into the polymer materials that are used for this purpose. The compounds according to the invention can be used alone or to achieve additive or synergistic actions in combination with other principles and classes of substances that can be used in tumor therapy.

As examples, there can be mentioned the combination with

Platinum complexes, such as, e.g., cis-platinum, carboplatinum,

intercalating substances, e.g., from the class of anthracyclines, such as, e.g., doxorubicin, or from the class of anthrapyrazoles, such as, e.g., CI-941,

substances that interact with tubulin, e.g., from the class of vinca-alkaloids, such as, e.g., vincristine, or vinblastine, or from the class of taxanes, such as, e.g., taxol, or taxotere, or from the class of macrolides, such as, e.g., rhizoxin or other compounds, such as, e.g., colchicine, combretastatin A-4, discodermolide and its analogs,

DNA topoisomerase inhibitors, such as, e.g., camptothecin, etoposide, topotecan, teniposide, folate- or pyrimidine-antimetabolites, such as, e.g., lometrexol, gemcitubin,

DNA-alkylating compounds, such as, e.g., adozelesin, dystamycin A,

inhibitors of growth factors (e.g., of PDGF, EGF, TGFb, EGF), such as, e.g., somatostatin, suramin, or bombesin antagonists,

inhibitors of protein tyrosine kinases or protein kinases A or C, such as, e.g., erbstatin, genistein, staurosporine, ilmofosine, 8-CI-cAMP,

antihormones from the class of antigestagens, such as, e.g., mifepristone, onapristone or from the class of antiestrogens, such as, e.g., tamoxifen or from the class of antiandrogens, such as, e.g., cyproterone acetate,

metastases-inhibiting compounds, e.g., from the class of eicosanoids, such as, e.g., PGl₂, PGE₁, 6-oxo-PGEI as well as their more stable derivatives (e.g., iloprost, cicaprost, or misoprostol),

inhibitory, oncogenic RAS proteins, which influence the mitotic signal transduction, such as, for example, inhibitors of the farnesyl-protein-transferase,

natural or synthetically produced antibodies, which are directed against factors or their receptors, which promote tumor growth, such as, for example, the erbB2 antibodies.

The invention also relates to pharmaceutical agents that are based on pharmaceutically compatible compounds, i.e., compounds of general formula I that are nontoxic in the doses used, optionally together with commonly used adjuvants and vehicles.

The compounds according to the invention can be encapsulated with liposomes or enclosed in an α-, β-, or γ-cyclodextrin clathrate.

According to methods of galenicals that are known in the art, the compounds according to the invention can be processed into pharmaceutical preparations for enteral, percutaneous, parenteral or local administration. They can be administered in the form of tablets, coated tablets, gel capsules, granulates, suppositories, implants, injectable, sterile, aqueous or oily solutions, suspensions or emulsions, ointments, creams and gels.

In this case, the active ingredient or ingredients can be mixed with the adjuvants that are commonly used in galenicals, such as, e.g., gum arabic, talc, starch, mannitol, methyl cellulose, lactose, surfactants such as Tweens or Myij, magnesium stearate, aqueous or non-aqueous vehicles, paraffin derivatives, cleaning agents, dispersing agents, emulsifiers, preservatives and flavoring substances for taste correction (e.g., ethereal oils).

The invention thus also relates to pharmaceutical compositions, which as active ingredients contain at least one compound according to the invention. A dosage unit contains about 0.1-100 mg of active ingredient(s). In humans, the dosage of the compounds according to the invention is approximately 0.1-1000 mg per day.

The examples below are used for a more detailed explanation of the invention, without intending that it be limited to these examples:

### EXAMPLE 1

4S,7R,8S,9R,13(Z),16S(Z))-4,8-Dihydroxy-1,10-dioxa-16-(fluoro-2-(2-methyl-4-thiazolyl)ethenyl)-5,5,7,9,13-pentamethylcyclohexadec-13-ene-2,6-dione

### Example 1a

(R)-1-(Tetrahydro-2H-pyran-2-yl(oxy))-propan-2-ol

A solution of 5 g (65.70 mmol) of R-1,2-propanediol, 6.15 ml (68 mmol) of 3,4-dihydro-2H-pyran and 0.2 g of p-toluenesulfonic acid-pyridinum salt in 100 ml of dichloromethane is stirred for 20 hours at 25° C. Then, it is neutralized by adding triethylamine, and then the reaction solution is concentrated by evaporation in a vacuum. After column chromatography on silica gel with a mixture that consists of ethyl acetate/hexane, 7.08 g (44.18 mmol; 67%) of 1a is obtained.

¹H-NMR (CDCl₃): δ 1.13 (3H), 1.48-1.59 (4H), 1.70-1.90 (2H), 3.40-4.00 (5H), 4.55 (1H) ppm.

### Example 1b

(5R)-5-Methyl-4-oxa-6-(tetrahydro-2H-pyran-2-yl(oxy))hexanoic Acid Ethyl Ester

A solution that consists of 7.08 g (44.18 mmol) of the compound that is described under la, 95 ml (877 mmol) of acrylic acid ethyl ester, 3.5 ml of aqueous tetrabutylammonium hydroxide solution (10%), and 180 ml of 50% aqueous sodium hydroxide solution in 300 ml of toluene is stirred for 2 hours at 25° C. Then, the solution is poured onto ice water. It is extracted with ethyl acetate, the organic phase is washed with saturated aqueous sodium chloride solution, dried on sodium sulfate and concentrated by evaporation in a vacuum. Column chromatography of the crude product that is obtained on silica gel with a mixture that consists of ethyl acetate/hexane yields 7.704 g (29.60 mmol; 67%) of lb.

¹H-NMR (CDCl₃): δ=1.12-1.20 (3H), 1.27 (3H), 1.48-1.90 (6H), 2.58 (2H), 3.30-4.00 (7H), 4.15 (2H), 4.60+4.70 (1H) ppm.

### Example 1c

(5R)-5-Methyl-4-oxa-6-(tetrahydro-2H-pyran-2-yl(oxy))hexan-1-ol

A solution of 7.704 g (29.60 mmol) of 1b in 70 ml of tetrahydrofuran is added in drops at 0° C. to a suspension of 1.7 g (44.80 mmol) of lithium aluminum hydride in 100 ml of tetrahydrofuran. It is allowed to stir for one more hour at 0° C., and then 10 ml of saturated aqueous ammonium chloride solution is added. Then, it is filtered on Celite and concentrated by evaporation in a vacuum. Column chromatography of the crude product that is obtained on silica gel with a mixture that consists of ethyl acetate/hexane yields 6.204 g (28.41 mmol; 96%) of 1c.

¹H-NMR (CDCl₃): δ=1.10-1.22 (3H), 1.45-1.90 (8H), 2.95 (1H), 3.30-4.05 (8H), 4.58-4.70 (1H) ppm.

### Example 1d

(5R)-5-Methyl-4-oxa-6-(tetrahydro-2H-pyran-2-yl(oxy))hexan-1-al

A solution of 5.99 ml (85.25 mmol) of dimethyl sulfoxide in 10 ml of dichloromethane is added to a solution of 3.67 ml (42.62 mmol) of oxalyl chloride in 100 ml of anhydrous dichloromethane at −70° C. It is stirred for 3 more minutes at −70° C., and then a solution of 6.204 g (28.41 mmol) of 1c in 100 ml of dichloromethane is added. It is allowed to stir for another 30 minutes at −70° C. Then, it is mixed with 31.5 ml (227.36 mmol) of triethylamine and allowed to react for 30 minutes at −50° C. Then, the reaction mixture is poured onto saturated aqueous sodium bicarbonate solution. It is extracted with dichloromethane. The organic phase is washed with saturated aqueous sodium chloride solution and dried on sodium sulfate. The crude product that is obtained (6.15 g, 100%) is used without purification in the next step.

### Example 1e

(6R)-6-Methyl-5-oxa-7-(tetrahydro-2H-pyran-2-yl(oxy))heptan-2-ol

19 ml (57 mmol) of a 3 molar solution of methylmagnesium chloride in tetrahydrofuran is diluted with 80 ml of tetrahydrofuran. Then, it is cooled to 0° C., and a solution of 6.15 g (28.41 mmol) of the compound, described under 1d, in 70 ml of tetrahydrofuran is added. It is stirred for 30 more minutes at 0° C., and then the reaction mixture is poured onto saturated aqueous ammonium chloride solution. Then, it is extracted with ethyl acetate. The organic phase is washed with saturated aqueous sodium chloride solution and dried on magnesium sulfate. Column chromatography of the crude product that is obtained on silica gel with a mixture that consists of ethyl acetate/hexane yields 6.008 g (25.86 mmol; 91%) of 1e.

¹H-NMR (CDCl₃): δ=1.12-1.22 (6H), 1.50-1.90 (8H), 3.32-4.07 (8H), 4.58-4.69 (1H) ppm.

### Example 1f

(6R)-6-Methyl-5-oxa-7-(tetrahydro-2H-pyran-2-yl(oxy))heptan-2-one

A solution of 6.008 g (25.86 mmol) of the compound that is described under le, 5.38 g (46.02 mmol) of N-methylmorpholino-N-oxide and 407 mg (1.16 mmol) of tetrapropylammonium perruthenate in 200 ml of dichloromethane is mixed with a molecular sieve (4A, about 600 spheres). It is allowed to stir for 20 more hours at 25° C. Then, it is concentrated by evaporation in a vacuum. The crude product that is obtained is purified by column chromatography on silica gel with a mixture that consists of ethyl acetate/hexane. 5.892 g (25.60 mmol; 99%) of If is obtained.

¹H-NMR(CDCl₃): δ=1.11-1.18 (3H), 1.45-1.88 (6H), 2.18 (3H), 2.67 (2H), 3.30-3.98 .(7H), 4.59+4.70 (1H) ppm.

### Example 1g

(2R,6Z,9S,10Z)-2,6-Dimethyl-9-[[dimethyl(1, 1-dimethylethyl)silyl]oxy]-10-fluoro-11-(2-methyl-4-thiazolyl)-3-oxa-undeca-6,10-dien-1-ol-tetrahydropyan-2-yl-ether (A) (2R,6E,9S,10Z)-2,6-dimethyl-9-[[dimethyl(1,1-dimethylethyl)silyl]oxy]-10-fluoro-11-(2-methyl-4-thiazolyl)-3-oxa-undeca-6,10-dien-1-ol-tetrahydropyran-2-yl-ether (B)

A solution of butyllithium in hexane is added in drops (3.73 ml; 9.33 mmol; 2.5 M) to a suspension of 4.498 g (7.81 mmol) of (3S,4Z)-5-(2-methylthiazol-4-yl)-3-(tert-butyl-dimethylsilyloxy)-4-fluoro-4-pentene-triphenylphosphonium iodide in 35 ml of tetrahydrofuran at 0° C. It is allowed to stir for 30 more minutes, and then a solution of 1.5 g (6.51 mmol) of the compound, described under if, in 35 ml of tetrahydrofuran is added. Then, it is stirred for 3 more hours at 0° C. Then, the reaction mixture is poured onto saturated aqueous ammonium chloride solution. It is extracted with ethyl acetate, the organic phase is washed with saturated aqueous sodium chloride solution and dried on sodium sulfate. The crude product that is obtained is purified by column chromatography on silica gel with a mixture that consists of ethyl acetate/hexane. 1.043 g (1.98 mmol; 30%) of title compound A and 870 mg (1.65 mmol; 25%) of title compound B are obtained.

Compound A: ¹H-NMR (CDCl₃): δ=0.07 (6H), 0.90 (9H), 1.12-1.20 (3H), 1.45-1.67 (5H), 1.71 (3H), 1.81 (1H), 2.24-2.51 (4H), 2.70 (3H), 3.30-3.77 (5H), 3.81-4.00 (1H), 4.17-4.27 (1H), 4.61 (1H), 5.23 (1H), 5.99-6.12 (1H), 7.34 (1H) ppm.

Compound B: ¹H-NMR (CDCl₃): δ=0.09 (6H), 0.90 (9H), 1.11-1.20 (3H), 1.48-1.61 (4H), 1.62 (3H), 1.68-1.90 (2H), 1.22-1.32 (2H), 1.39-1.47 (2H), 2.70 (3H), 3.28-3.65 (5H), 3.80-3.99 (1H), 1.16-4.27 (1H), 4.61 (1H), 5.21 (1H), 5.98-6.12 (1H), 7.33 (1H) ppm.

### Example 1h

(2R,6Z,9S,10Z)-2,6-Dimethyl-9-[[dimethyl(1,1-dimethylethyl)silyl]oxy]-10-fluoro-11-(2-methyl-4-thiazolyl)-3-oxa-undeca-6,10-dien-1-ol

A solution of 1.043 g (1.98 mmol) of compound A that is described under 1g, and 990 mg (3.94 mmol) of p-toluenesulfonic acid-pyridinium salt in 50 ml of ethanol is stirred for 2 hours at 50° C. Then, it is diluted with dichloromethane. The organic phase is washed with saturated aqueous sodium bicarbonate solution and with saturated aqueous sodium chloride solution. It is dried on sodium sulfate. The crude product that is obtained is purified by column chromatography on silica gel with a mixture that consists of ethyl acetate/hexane. 702 mg (1.58 mmol; 80%) of 1 h is obtained.

¹H-NMR (CDCl₃): δ=0.09 (6H), 0.91 (9H), 1.10 (3H), 1.72 (3H), 2.28-2.37 (2H), 2.40-2.51 (2H), 2.70 (3H), 3.35-3.65 (5H), 4.17-4.28 (1H), 5.28 (1H), 6.00-6.13 (1H), 7.34 (1H) ppm.

### Example 1i

(2R,6Z,9S,10Z)-2,6-Dimethyl-9-[[dimethyl(1,1-dimethlethyl)silyl]oxy]-10-fluoro-11-(2-methyl-4-thiazolyl)-3-oxa-undeca-6,10-dien-1-a1

Analogously to Example 1d, 698 mg (1.58 mmol; 100%) of crude Ii is obtained from 702 mg (1.58 mmol) of 1 h, which is used without purification in the next step.

¹H-NMR (CDCl₃): δ=0.07 (6H), 0.88 (9H), 1.25 (3H), 1.70 (3H), 2.23-2.48 (4H), 2.69 (3H), 3.44-3.59 (2H), 3.72 (1H), 4.14-4.26 (1H), 5.25 (1H), 5.98-6.11 (1H), 7.31 (1H), 9.61 (1H) ppm.

### Example 1k

(3S,6R,7S,8R,12Z,15S,16Z)-16-Fluoro-17-(2-methyl-4-thiazolyl)-9-oxa-5-oxo-4,4,6,8,12-pentamethyl-1,3,15-tris[[dimethyl(1,1-dimethylethy)silyl]oxy]heptadeca-12,16-dien-7-ol (A) (3S,6S,7R,8R,12Z,15S,16Z)-16-fluoro-17-(2-methyl-4-thiazolyl)-9-oxa-5-oxo-4,4,6,8,12-pentamethyl-1,3,7,15-tris[[dimethyl(1,1-dimethylethyl)silyl]oxy]heptadeca-12,16-dien-7-ol (B)

Lithium diisopropylamide is produced in 20 ml of absolute tetrahydrofuran from 473 mg (3.37 mmol) of diisopropylamine and 1.37 ml (3.41 mmol) of a 2.5 molar solution of butyllithium in hexane. A solution of 1.273 g (3.16 mmol) of(3S)-1,3-bis[[dimethyl(1,1-dimethylethyl)silyl]oxy]-4,4-dimethylheptan-5-one in 15 ml of tetrahydrofuran is then added at −70° C., and it is allowed to stir for one more hour at −40 to −30° C. Then, it is cooled again to −70° C., and a solution of 698 mg (1.58 mmol) of 1i in 15 ml of tetrahydrofuran is slowly added in drops. It is allowed to stir for one more hour at −70° C., and then the reaction mixture is poured onto saturated aqueous ammonium chloride solution. It is extracted with ethyl acetate, the organic phase is washed with saturated aqueous sodium chloride solution and dried on sodium sulfate. The crude product that is obtained is purified by column chromatography on silica gel with a mixture that consists of ethyl acetate/hexane. 494 mg (0.59 mmol; 37%) of title compound A and 464 mg (0.55 mmol; 35%) of title compound B are obtained.

Compound A: ¹H-NMR (CDCl₃): δ=0.00-0.18 (18H), 0.84-0.99 (27H), 1.05 (3H), 1.08-1.18 (6H), 1.21 (3H), 1.71 (3H), 2.20-2.47 (4H), 2.69 (3H), 3.18-3.36 (3H), 3.50-3.70 (4H), 3.90 (1H), 4.15-4.28 (1H), 5.24 (1H), 5.98-6.12 (1H), 7.32 (1H) ppm.

Compound B: ¹H-NMR (CDCl₃): δ=0.02-0.15 (18H), 0.85-0.94 (27H), 1.05 (3H), 1.08 (3H), 1.15 (3H), 1.20 (3H), 1.75 (3H), 2.30 (2H), 2.37-2.52 (2H), 2.70 (3H), 3.20-3.74 (7H), 4.09 (1H), 4.17-4.26 (1H), 5.25 (1H), 6.00-6.14 (1H), 7.34 (1H) ppm.

### Example 11

(3S,6R,7S,8R,12Z,15S,16Z)-16-Fluoro-17-(2-methyl-4-thiazolyl)-9-oxa-5-oxo-4,4,6,8,12-pentamethyl-1,3,7,15-tetrakis[[dimethyl(1,1-dimethylethyl)silyl]oxy]heptadeca-12,16-diene

135 μl (1.17 mmol) of 2,6-lutidine and 161 μl (0.70 mmol) of trifluoromethanesulfonic acid-tert-butyldimethylsilyl ester are added to a solution of 494 mg (0.59 mmol) of compound A, described under 1k, in 30 ml of dichloromethane at −10° C. It is allowed to stir for 2 more hours at 0° C. Then, the reaction mixture is poured onto saturated aqueous sodium bicarbonate solution. It is extracted with dichloromethane, the organic phase is washed with saturated aqueous sodium chloride solution, dried on sodium sulfate and concentrated by evaporation in a vacuum. The crude product that is obtained is purified by column chromatography on silica gel with a mixture that consists of ethyl acetate/hexane. 527 g (0.55 mmol; 93%) of 11 is obtained.

¹H-NMR (CDCl₃): δ=0.00-0.15 (24H), 0.82-0.97 (36H), 1.03 (3H), 1.06 (3H), 1.11 (3H), 1.28 (3H), 1.69 (3H), 2.22-2.46 (4H), 2.70 (3H), 3.18-3.40 (4H), 3.52-3.72 (2H), 3.80 (1H), 3.99 (1H), 4.13-4.27 (1H), 5.22 (1H), 5.99-6.12 (1H), 7.33 (1H) ppm.

### Example 1m

(3S,6R,7S,8R,12Z,15S,16Z)-16-Fluoro-17-(2-methyl-4-thiazolyl)-9-oxa-5-oxo-4,4,6,8,12-pentamethyl-3,7,15-tris[[dimethyl(1,1-dimethylethyl)silyl]oxy]heptadeca-12,16-dien-1-ol

A solution of 527 mg (0.55 mmol) of 11 and 128 mg (0.55 mmol) of camphor-10-sulfonic acid in 20 ml of a 1:1 mixture that consists of dichloromethane and methanol is stirred for 2 hours at 25° C. Then, excess triethylamine is added, and it is concentrated by evaporation in a vacuum. The crude product that is obtained is purified by column chromatography on silica gel with a mixture that consists of ethyl acetate/hexane. 404 mg (0.48 mmol; 87%) of lm is obtained.

¹H-NMR (CDCl₃): δ=0.03-0.14 (18H), 0.85-0.95 (27H), 1.06 (3H), 1.08-1.15 (6H), 1.24 (3H), 1.71 (3H), 2.11-2.35 (3H), 2.42 (2H), 2.69 (3H), 3.15-3.40 (4H), 3.59-3.69 (2H), 3.99-4.06 (2H), 4.14-4.25 (1H), 5.21 (1H), 5.99-6.12 (1H), 7.34 (1H) ppm.

### Example 1n

(3S,6R,7S,8R,12Z,15S,16Z)-16-Fluoro-17-(2-methyl-4-thiazolyl)-9-oxa-5-oxo-4,4,6,8,12-pentamethyl-3,7,15-tris[[dimethyl(1,1-dimethylethyl)silyl]oxy]heptadeca-12,16-dien-1-al Analogously to Example 1d, 403 mg (0.48 mmol, 100%) of In is obtained from 404 mg (0.48 mmol) of the substance that is described under Example 1m. The substance is used without purification in the next step.

¹H-NMR (CDCl₃): δ=0.02-0.13 (18H), 0.83-0.92 (27H), 1.02 (3H), 1.09 (3H), 1.11 (3H), 1.39 (3H), 1.71 (3H), 2.10-2.31 (2H), 2.34-2.45 (3H), 2.58-2.63 (1H), 2.71 (3H), 3.14-3.40 (4H), 4.01 (1H), 4.14-4.26 (1H), 4.49 (1H), 5.22 (1H), 5.99-6.12 (1H), 7.34 (1H) ppm.

### Example 1o

(3S,6R,7S,8R,12Z,15S,16Z)-16-Fluoro-17-(2-methyl-4-thiazolyl)-9-oxa-5-oxo-4,4,6,8,12-pentamethyl-3,7,15-tris[[dimethyl(1,1-dimethylethyl)silyl]oxy]heptadeca-12,16-dienoic acid A solution of 403 mg (0.48 mmol) of the substance, described under In, in 15 ml of tert-butanol is mixed with 13.7 ml of 2-methyl-2-butene (27.4 mmol). It is then cooled to 2° C., and 3.7 ml of water, 198 mg (1.44 mmol) of sodium dihydrogen phosphate-monohydrate, and 336 mg of sodium chlorite (2.97 mmol) are added and allowed to stir for 1 more hour at 2° C. Then, it is poured into saturated sodium thiosulfate solution, diluted with water and extracted several times with ethyl acetate. The combined organic extracts are dried on sodium sulfate, and the residue that is obtained after filtration and removal of the solvent is purified by chromatography on fine silica gel with a gradient system that consists of n-hexane and ethyl acetate. 345 mg (0.40 mmol, 84%) of 1o is obtained.

¹H-NMR (CDCl₃): δ=0.04-0.15 (18H), 0.86-0.94 (27H), 1.05 (3H), 1.14 (3H), 1.18 (3H), 1.28 (3H), 1.71 (3H), 2.05 (3H), 2.26-2.48 (4H), 2.63-2.71 (1H), 2.72 (3H), 3.10-3.42 (4H), 4.08 (1H), 4.13-4.26 (1H), 4.37 (1H), 5.23 (1H), 6.20-6.33 (1H), 7.33 (1H) ppm.

### Example 1p

(3S,6R,7S,8R,12Z,15S,16Z)-3,7-Bis[[dimethyl(1,1-dimethylethyl)silyl]oxy]-16-fluoro-15-hydroxy-17-(2-methyl-4-thiazolyl)-9-oxa-5-oxo-4,4,6,8,12-pentamethyl-12,16-dienoic acid

A solution of 345 mg (0.40 mmol) of 1o in 15 ml of tetrahydrofuran is mixed with 6 ml of a 1 molar solution of tetrabutylammonium fluoride in tetrahydrofuran. It is allowed to stir for one more hour at 25° C., and then the reaction mixture is poured onto ice-cold, saturated, aqueous ammonium chloride solution. It is extracted with ethyl acetate, and the organic phase is washed with 1N hydrochloric acid and saturated aqueous sodium bicarbonate solution. Then, it is dried on sodium sulfate. The crude product that is obtained (299 mg; 0.40 mmol; 100%) is used without purification in the next step.

¹H-NMR (CDCl₃): δ=0.03-0.13 (12H), 0.86-0.92 (18H), 1.06 (3H), 1.11 (3H), 1.16 (3H), 1.28 (3H), 1.73 (3H), 2.27-2.59 (6H), 2.71 (3H), 3.08-3.17 (1H), 3.30-3.49 (3H), 4.08 (1H), 4.21-4.30 (1H), 4.37 (1H), 5.28 (1H), 6.28-6.42 (1H), 7.33 (1H) ppm.

### Example 1q

4S,7R,8S,9R,13(Z),16S(Z))-4,8-Dihydroxy-1,10-dioxa-16-(1-fluoro-2-(2-methyl-4-thiazolyl)ethenyl)-5,5,7,9,13-pentamethylcyclohexadec-13-ene-2,6-dione

334 μl (2.40 mmol) of triethylamine and 315 μl (2.01 mmol) of 2,4,6-trichlorobenzoyl chloride are added to a solution of 299 mg (0.40 mmol) of the compound, described under 1p, in 4 ml of tetrahydrofuran. It is allowed to stir for 15 more minutes at 25° C. and then diluted with 35 ml of toluene. This solution is added in drops over 3 hours to a solution of 510 mg (4.18 mmol) of N,N-dimethylaminopyridine in 100 ml of toluene. After the addition is completed, it is stirred for another hour at 25° C. Then, the reaction mixture is concentrated by evaporation in a vacuum. After column chromatography, 169 mg (0.23 mmol, 58%) of the title compound is obtained.

¹H-NMR (CDCl₃): δ=0.02-0.14 (12H), 0.85-0.93 (18H), 1.07 (3H), 1.12 (3H), 1.19-1.24 (6H), 1.67 (3H), 2.00-2.10 (1H), 2.41-2.65 (3H), 2.70 (3H), 2.76-2.88 (1H), 3.14-3.23 (1H), 3.39-3.53 (3H), 4.02 (1H), 4.34 (1H), 5.23 (1H), 5.46-5.56 (1H), 6.09-6.12 (1H), 7.38 (1H) ppm.

### EXAMPLE 1

4S,7R,8S,9R,13(Z),16S(Z))-4,8-Bis[[dimethyl(1,1-dimethylethyl)silyl]oxy]-1,10-dioxa-16-(1-fluoro-2-(2-methyl-4-thiazolyl)ethenyl)-5,5,7,9,13-pentamethylcyclohexadec-13-ene-2,6-dione 530 μl of HF-pyridine complex is added to a solution of 169 mg (0.23 mmol) of the compound, described under 1q, in 10 ml of tetrahydrofuran at 0° C. It is stirred for one hour at 25° C., and then 530 μl of HF-pyridine complex is added again. Then, it is allowed to stir for 10 more hours at 25° C. Then, the reaction mixture is poured onto saturated aqueous sodium bicarbonate solution. It is extracted with dichloromethane, the organic phase is washed with saturated aqueous sodium chloride solution and dried on sodium sulfate. Column chromatography on silica gel with a mixture that consists of ethyl acetate/hexane yields 80 mg (0.16 mmol; 69%) of the title compound.

¹H-NMR (CDCl₃): δ=1.11 (3H), 1.19 (3H), 1.23 (3H), 1.31 (3H), 1.71 (3H), 2.06-2.17 (1H), 2.38-2.68 (4H), 2.70 (3H), 2.73-2.87 (1H), 3.00 (1H), 3.19-3.31 (2H), 3.48 (1H), 3.74-3.84 (2H), 4.12-4.22 (1H), 5.38-5.49 (1H), 6.10-6.13 (1H), 7.38 (1H) ppm.

### EXAMPLE 2

(1S,3S(Z),7S,10R,11S,12R,16R)-7,11-Dihydroxy-3-(1-fluoro-2-(2-methyl-4-thiazolyl)ethenyl)-8,8,10,12,16-pentamethyl-4,13,17-trioxabicyclo[14.1.0]heptadecane-5,9-dione (A) (1R,3S(Z),7S,10R,11S,12R,16S)-7,11-Dihydroxy-3-(1-fluoro-2-(2-methyl-4-thiazolyl)ethenyl)-8,8,10,12,16-pentamethyl-4,13,17-trioxabicyclo[14.1.0]heptadecane-5,9-dione (B) A solution of 20 mg (0.04 mmol) of the compound, described under Example 1, in 2 ml of acetonitrile is mixed with 237 μl of a 1 M solution of sodium-ethylenediamine-tetraacetate. It is cooled to 0° C., and then 440 μl (4.91 mmol) of 1,1,1-trifluoroacetone as well as a mixture that consists of 121 mg (0.20 mmol) of oxone and 28 mg (0.33 mmol) of sodium bicarbonate are added. It is allowed to stir for 2 more hours at 2° C., and then it is poured onto sodium thiosulfate solution. It is extracted with ethyl acetate, the organic phase is washed with saturated aqueous sodium chloride solution and dried on sodium sulfate. After column chromatography on silica gel with a mixture that consists of ethyl acetate/hexane, 10 mg (0.019 mmol; 49%) of title compound A as well as 5 mg (0.01 mmol; 24%) of title compound B are obtained.

Compound A: ¹H-NMR (CDCl₃): δ=1.02 (3H), 1.11 (3H), 1.24 (3H), 1.30 (3H), 1.40 (3H), 1.63-1.74 (1H), 1.78-1.86 (1H), 1.99-2.08 (1H), 2.23-2.31 (1H), 2.50-2.56 (1H), 2.61-2.68 (1H), 2.72 (3H), 2.93 (1H), 3.43-3.59 (4H), 3.60=3.66 (1H), 3.72-3.78 (1H), 4.20 (1H), 4.56 (1H), 5.70-5.77 (1H), 6.21-6.32 (1H), 7.38 (1H) ppm.

Compound B: ¹H-NMR (CDCl₃): δ=1.07 (3H), 1.14 (3H), 1.21 (3H), 1.27 (3H), 1.31 (3H), 1.72-1.81 (1H), 1.83-1.91 (1H), 2.08-2.17 (1H), 2.23-2.31 (1H), 2.57-2.65 (2H), 2.71 (3H), 2.89 (1H), 3.00 (1H), 3.46-3.58 (1H), 3.65 (1H), 3.83-3.90 (1H), 4.18 (1H), 5.78-5.86 (1H), 6.18-6.28 (1H), 7.40 (1H) ppm.

### EXAMPLE 3

4S,7R,8S,9R,13(E),16S(Z))-4,8-Dihydroxy-1,10-dioxa-16-(1-fluoro-2-(2-methyl-4-thiazolyl)ethenyl)-5,5,7,9,13-pentamethylcyclohexadec-13-ene-2,6-dione

### Example 3a

(2R,6E,9S,10Z)-2,6-Dimethyl-9-[[dimethyl(1,1-dimethylethyl)silyl]oxy]-10-fluoro-11-(2-methyl-4-thiazolyl)-3-oxa-undeca-6,10-dien-1-ol

Analogously to Example 1h, 600 mg (1.35 mmol; 82%) of the title compound is obtained from 870 mg (1.65 mmol) of compound B that is described under Example 1g.

¹H-NMR (CDCl₃): δ 0.08-0.12 (6H), 0.91 (9H), 1.09 (3H), 1.63 (3H), 2.27 (2H), 2.44 (2H), 2.70 (3H), 3.37-3.68 (5H), 4.17-4.29 (1H), 5.23 (1H), 5.98-6.12 (1H), 7.33 (1H) ppm.

### Example 3b

(2R,6E,9S,10Z)-2,6-Dimethyl-9-[[dimethyl(1,1-dimethylethyl)silyl]oxy]-10-fluoro-11-(2-methyl-4-thiazolyl)-3-oxa-undeca-6,10-dien-1-al

Analogously to Example 1d, 596 mg (1.35 mmol, 100% crude) of the title compound is obtained from 600 mg (1.35 mmol) of the compound that is described under 3a.

### Example 3c

(3S,6R,7S,8R,12E,15 S,16Z)-16-Fluoro-17-(2-methyl-4-thiazolyl)-9-oxa-5-oxo-4,4,6,8,12-pentamethyl-1,3,15-tris[[dimethyl(1,1-dimethylethyl)silyl]oxy]heptadeca-12,16-dien-7-ol (A) (3S,6S,7R,8R,12E,15S,16Z)-16-Fluoro-17-(2-methyl-4-thiazolyl)-9-oxa-5-oxo-4,4,6,8,12-pentamethyl-1,3,7,15-tris[[dimethyl(1,1-dimethylethyl)silyl]oxy]heptadeca-12,16-dien-7-ol (B)

Analogously to Example 1k, 464 mg (0.55 mmol; 41%) of title compound A and 388 mg (0.46 mmol; 34%) of title compound B are obtained from 596 mg (1.35 mmol) of the compound that is described under 3b.

Compound A: ¹H-NMR (CDCl₃): δ 0.00-0.16 (18H), 0.86-0.99 (27H), 1.05 (3H), 1.11 (3H), 1.15 (3H), 1.22 (3H), 1.62 (3H), 2.25 (2H), 2.41 (2H), 2.53 (1H), 2.69 (3H), 3.18-3.37 (3H), 3.48-3.73 (4H), 3.90 (1H), 4.15-4.28 (1H), 5.21 (1H), 5.98-6.10 (1H), 7.33 (1H) ppm.

Compound B: ¹H-NMR (CDCl₃): δ=0.00-0.18 (18H), 0.84-0.97 (27H), 1.03 (3H), 1.08 (3H), 1.17 (3H), 1.19 (3H), 2.24 (2H), 2.43 (2H), 2.70 (3H), 3.18-3.28 (2H), 3.42-3.52 (2H), 3.57-3.73 (3H), 4.07 (1H), 4.16-4.28 (1H), 5.22 (1H), 5.99-6.12 (1H), 7.32 (1H) ppm.

### Example 3d

(3S,6R,7S,8R,12E,15S,16Z)-16-Fluoro-17-(2-methyl-4-thiazolyl)-9-oxa-5-oxo-4,4,6,8,12-pentamethyl-1,3,7,15-tetrakis[[dimethyl(1,1-dimethylethyl)silyl]oxy]heptadeca-12,16-diene

Analogously to Example 11, 485 mg (0.51 mmol, 92%) of the title compound is obtained from 464 mg (0.55 mmol) of compound A that is described under 3c.

¹H-NMR (CDCl₃): δ=−0.02-0.13 (24H), 0.82-0.96 (36H), 0.98-1.04 (6H), 1.10 (3H), 1.28 (3H), 1.62 (3H), 2.17 (2H), 2.40 (2H), 2.69 (3H), 3.20 (1H), 3.28-3.39 (3H), 3.52-3.72 (2H), 3.80 (1H), 3.98 (1H), 4.01-4.26 (1H), 5.18 (1H), 5.98-6.11 (1H), 7.31 (1H) ppm.

### Example 3e

(3S,6R,7S,8R,12E,15S,16Z)-16-Fluoro-17-(2-methyl-4-thiazolyl)-9-oxa-5-oxo-4,4,6,8,12-pentamethyl)-3,7,15-tris[[dimethyl(1,1-dimethylethyl)silyl]oxy]heptadeca-12,16-dien-1-ol

Analogously to Example 1m, 370 mg (0.44 mmol, 86%) of the title compound is obtained from 485 mg (0.51 mmol) of the compound that is described under 3d.

¹H-NMR (CDCl₃): δ=0.02-0.17 (18H), 0.84-0.97 (27H), 1.04 (3H), 1.07-1.14 (6H), 1.22 (3H), 1.61 (3H), 2.17 (2H), 2.41 (2H), 2.70 (3H), 3.20 (1H), 3.30-3.42 (3H), 3.59-3.70 (2H), 4.02 (2H), 4.13-4.29 (1H), 5.18 (1H), 5.98-6.10 (1H), 7.32 (1H) ppm.

### Example 3f

(3S,6R,7S,8R,12E,15S,16Z)-16-Fluoro-17-(2-methyl-4-thiazolyl)-9-oxa-5-oxo-4,4,6,8,12-pentamethyl-3,7,15-tris[[dimethyl(1,1-dimethylethyl)silyl]oxy]heptadeca-12,16-dien-1-al

Analogously to Example 1d, 370 mg (0.44 mmol, 100% crude) of the title compound is obtained from 370 mg (0.44 mol) of the compound that is described under 3e.

¹H-NMR (CDCl₃): δ=0.01-0.15 (18H), 0.82-0.95 (27H), 1.01 (3H), 1.05-1.12 (6H), 1.27 (3H), 1.61 (3H), 2.15 (2H), 2.42 (2H), 2.56-2.67 (1H), 2.70 (3H), 3.17 (1H), 3.28-3.41 (3H), 4.00 (1H), 4.13-4.28 (1H), 4.40 (1H), 5.18 (1H), 5.98-6.11 (1H), 7.32 (1H) ppm.

### Example 3g

(3S,6R,7S,8R,12E,15S,16Z)-16-Fluoro-17-(2-methyl-4-thiazolyl)-9-oxa-5-oxo-4,4,6,8,12-pentamethyl-3,7,15-tris[[dimethyl(1,1-dimethlethyl)silyl]oxy]heptadeca-12,16-dienoic Acid

Analogously to Example 1o, 302 mg (0.35 mmol, 80%) of the title compound is obtained from 370 mg (0.44 mmol) of the compound that is described under 3f.

¹H-NMR (CDCl₃): δ=0.00-0.16 (18H), 0.82-0.98 (27H), 1.05 (3H), 1.10 (3H), 1.15 (3H), 1.21 (3H), 1.61 (3H), 2.15 (2H), 2.25-2.53 (3H), 2.63-2.76 (1H), 2.72 (3H), 3.17 (1H), 3.28-3.44 (3H), 4.07 (1H), 4.16-4.28 (1H), 4.34 (1H), 5.21 (1H), 6.07-6.20 (1H), 7.35 (1H) ppm.

### Example 3h

(3S,6R,7S,8R,12E,15S,16Z)-3,7-Bis[[dimethyl(1,1-dimethylethyl)silyl]oxy]-16-fluoro-15-hydroxy-17-(2-methyl-4-thiazolyl)-9-oxa-5-oxo-4,4,6,8,12-pentamethyl-12,16-dienoic Acid

Analogously to Example 1p, 260 mg (0.35 mmol, 100% crude) of the title compound is obtained from 302 mg (0.35 mmol) of the compound that is described under 3 g.

¹H-NMR (CDCl₃): δ=0.02-0.13 (12H), 0.83-0.98 (18H), 1.04 (3H), 1.08-1.17 (6H), 1.24 (3H), 1.63 (3H), 2.16 (1H), 2.22-2.35 (1H), 2.42-2.69 (3H), 2.69 (3H), 3.11 (1H), 3.30-3.47 (3H), 3.99-4.14 (1H), 3.27-3.47 (2H), 5.22 (1H), 6.18-6.32 (1H), 7.33 (1H) ppm.

### Example 3i

4S,7R,8S,9R,13(E),16S(Z))-4,8-Dihydroxy-1,10-dioxa-16-(1-fluoro-2-(2-methyl-4-thiazolyl)ethenyl)-5,5,7,9,13-pentamethylcyclohexadec-13-ene-2,6-dione

Analogously to Example 1q, 175 mg (0.24 mmol, 69%) of the title compound is obtained from 260 mg (0.35 mmol) of the compound that is described under 3 h.

¹H-NMR (CDCl₃): δ=0.03-0.16 (12H), 0.85-0.94 (18H), 1.06 (3H), 1.09-1.16 (6H), 1.18 (3H), 1.63 (3H), 2.02-2.29 (2H), 2.38-2.46 (1H), 2.53-2.63 (2H), 2.67-2.82 (1H), 2.68 (3H), 3.08 (1H), 3.33-3.48 (2H), 3.55-3.62 (1H), 4.00 (1H), 4.43 (1H), 5.29 (1H), 5.46-5.57 (1H), 6.12-6.24 (1H), 7.38 (1H) ppm.

### EXAMPLE 3

4S,7R,8S,9R,13(E),16S(Z))-4,8-Dihydroxy-1,10-dioxa-16-(1-fluoro-2-(2-methyl-4-thiazolyl)ethenyl)-5,5,7,9,13-pentamethylcyclohexadec-13-ene-2,6-dione

Analogously to Example 1, 85 mg (0.17 mmol, 71%) of the title compound is obtained from 175 mg (0.24 mmol) of the compound that is described under 3i.

¹H-NMR (CDCl₃): δ=1.06 (3H), 1.17 (3H), 1.24 (3H), 1.30 (3H), 1.69 (3H), 2.23 (2H), 2.48-2.61 (2H), 2.61-2.77 (2H), 2.70 (3H), 3.28-3.45 (3H), 3.52 (1H), 3.67-3.79 (2H), 4.21 (1H), 5.23 (1H), 5.53-5.63 (1H), 6.12-6.26 (1H), 7.39 (1H) ppm.

### EXAMPLE 4

(1S,3S(Z),7S,10R,11S,12R,16S)-7,11-Dihydroxy-3-(1-fluoro-2-(2-methyl-4-thiazolyl)ethenyl)-8,8,10,12,16-pentamethyl-4,13,17-trioxabicyclo[14.1.0]heptadecane-5,9-dione (A) (1R,3S(Z),7S,10R,11S,12R,16R)-7,11-Dihydroxy-3-(1-fluoro-2-(2-methyl-4-thiazolyl)ethenyl)-8,8,10,12,16-pentamethyl-4,13,17-trioxabicyclo[14.1.0]heptadecane-5,9-dione (B) Analogously to Example 2, 19 mg (0.037 mmol, 37%) of title compound A and 14 mg (0.027 mmol, 27%) of title compound B are obtained from 50 mg (0.10 mmol) of the compound that is described under Example 3.

Compound A: ¹H-NMR(CDCl₃): δ=1.11 (3H), 1.17-1.25 (6H), 1.28 (3H), 1.36 (3H), 1.52-1.61 (1H), 2.08-2.22 (3H), 2.45 (1H), 2.69 (3H), 2.76-2.85 (1H), 2.98-3.08 (2H), 3.17-3.37 (2H), 3.46-3.60 (2H), 3.69 (1H), 4.31 (1H), 5.61-5.73 (1H), 6.16-6.28 (1H), 7.39 (1H) ppm.

Compound B: ¹H-NMR (CDCl₃): δ=1.02 (3H), 1.17 (3H), 1.22 (3H), 1.29 (3H), 1.36 (3H), 1.55-1.79 (3H), 1.95-2.35 (3H), 2.47-2.63 (2H), 2.69 (3H), 2.94 (1H), 3.09 (1H), 3.16-3.27 (1H), 3.38-3.48 (1H), 3.54-3.69 (3H), 4.16 (1H), 4.32 (1H), 5.62-5.73 (1H), 6.19-6.32 (1H), 7.39 (1H) ppm.

1. Epothilone derivatives of general formula I, 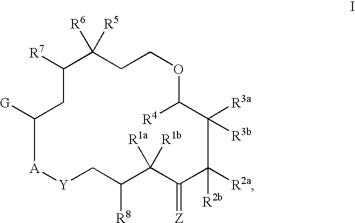
in which
R^{1a}, R^{1b} are the same or different and mean hydrogen, C₁-C₁₀-alkyl, aryl, C₇-C₂₀-aralkyl, or together a —(CH₂)ₘ group with m=1, 2, 3, 4 or 5, or a —CH₂—O—CH₂ group,
R^{2a}, R^{2b} are the same or different and mean hydrogen, C₁-C₁₀-alkyl, aryl, C₇-C₂₀-aralkyl, (CH₂)ᵣ—CδC—(CH₂)ₚ—R⁹, —(CH₂)CH═CH—(CH₂)ₚ—R⁹,
r is equal to 0 to 4,
p is equal to 0 to 3,
R⁹ is hydrogen, C₁-C₁₀-alkyl, aryl, C₇-C₂₀-aralkyl, C₁-C₁₀-acyl, or if p>0, also a group OR¹⁰,
R¹⁰ means hydrogen, or a protective group PG¹⁰,
R^{3a} means hydrogen, C₁-C₁₀-alkyl, aryl, C₇-C₂₀-aralkyl,
R^{3b} means OH, OPG³
R⁴ means hydrogen, C₁-C₁₀-alkyl, aryl, C₇-C₂₀-aralkyl,
R⁵ means hydrogen, C₁-C₁₀-alkyl, aryl, C₇-C₂₀-aralkyl, halogen, cyano, (CH₂)ₛ-T,
whereby s stands for 1, 2, 3, or 4,
T stands for OR¹¹ or Hal,
R¹¹ stands for hydrogen or PG¹¹,
R⁶, R⁷ each mean a hydrogen atom, together an additional bond or an oxygen atom,
G means a group 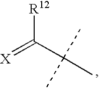
a bicyclic or tricyclic aryl radical,
R¹² means hydrogen, halogen, CN, C₁-C₂₀-alkyl, aryl, C₇-C₂₀-aralkyl, which can all be substituted,
X means an oxygen atom, two alkoxy groups OR¹³, a C₂-C₁₀-alkylene-α, ω-dioxy group, which can be straight-chain or branched, H/OR¹⁴ or a grouping CR¹⁵R¹⁶,
whereby
R¹³ stands for a C₁-C₂₀-alkyl radical,
R¹⁴ stands for hydrogen or a protective group PG¹⁴,
R¹⁵, R¹⁶ are the same or different and stand for hydrogen, a C₁-C₂₀-alkyl, aryl, or C₇-C₂₀-aralkyl radical,
A-Y means a group O—C(═O), O—CH₂, CH₂C(═O), NR¹⁷—C(═O), NR¹⁷—SO₂,
R¹⁷ means hydrogen, or C₁-C₁₀-alkyl,
Z means an oxygen atom or H/OR¹⁸,
whereby
R¹⁸ means hydrogen or a protective group PG¹⁸,
R⁸ means OH or OPG⁸,
Hal means halogen, preferably fluorine, chlorine or bromine, excluding those compounds in which R^{2a} is hydrogen, and R^{2b} means hydrogen, alkyl or aryl, and simultaneously
R⁵ means hydrogen, alkyl or aryl, and simultaneously
A-Y means a grouping O—C(═O), O—CH₂, or NR¹⁷—C(═O), and simultaneously
G means a bicyclic or tricyclic aryl radical or a grouping X═(CR¹²)—,
whereby all other radicals can have the indicated meanings.

2. Epothilone derivatives of general formula I according to claim 1, namely
(4S,7R,8S,9R,13(Z),16S(Z))-4,8-Dihydroxy-1,10-dioxa-16-(1-fluoro-2-(2-methyl-4-thiazolyl)ethenyl)-5,5,7,9,13-pentamethyl-cyclohexadec-13-ene-2,6-dione
(1S,3S(Z),7S,10R,11S,12R,16R)-7,11-Dihydroxy-3-(1-fluoro-2-(2-methyl-4-thiazolyl)ethenyl)-8,8,10,12,16-pentamethyl-4,13,17-trioxabicyclo[14.1.0]heptadecane-5,9-dione
(4S,7R,8S,9R,13(Z),16S(Z))-4,8-Dihydroxy-1,10-dioxa-7-ethyl-16-(1-fluoro-2-(2-methyl-4-thiazolyl)ethenyl)-5,5,9,13-tetramethyl-cyclohexadec-13-ene-2,6-dione
(1S,3S(Z),7S,10R,11S,12R,16R)-7,11-Dihydroxy-10-ethyl-3-(1-fluoro-2-(2-methyl-4-thiazolyl)ethenyl)-8,8,12,16-tetramethyl-4,13,17-trioxabicyclo[14.1.0]heptadecane-5,9-dione
(4S,7R,8S,9R,13(Z),16S(Z))-4,8-Dihydroxy-1,10-dioxa-16-(1-fluoro-2-(2-methyl-4-thiazolyl)ethenyl)-7-(prop-2-en-1-yl)-5,5,9,13-tetramethyl-cyclohexadec-13-ene-2,6-dione
(1S,3S(Z),7S,10R,11S,12R,16R)-7,11-Dihydroxy-3-(1-fluoro-2-(2-methyl-4-thiazolyl)ethenyl)-10-(prop-2-en-1-yl)-8,8,12,16-tetramethyl-4,13,17-trioxabicyclo[14.1.0]-heptadecane-5,9-dione
(4S,7R,8S,9R,13(Z),16S(Z))-4,8-Dihydroxy-1,10-dioxa-16-(1-fluoro-2-(2-methyloxazol-4-yl)ethenyl)-5,5,7,9,13-pentamethyl-cyclohexadec-13-ene-2,6-dione
(1S,3S(Z),7S,10R,11S,12R,16R)-7,11-Dihydroxy-3-(1-fluoro-2-(2-methyloxazol-4-yl)ethenyl)-8,8,10,12,16-pentamethyl-4,13,17-trioxabicyclo[14.1.0]heptadecane-5,9-dione
(4S,7R,8S,9R,13(Z),16S(Z))-4,8-Dihydroxy-1,10-dioxa-7-ethyl-16-(1-fluoro-2-(2-methyloxazol-4-yl)ethenyl)-5,5,9,13-tetramethyl-cyclohexadec-13-ene-2,6-dione
(1S,3S(Z),7S,10R,11S,12R,16R)-7,11-Dihydroxy-10-ethyl-3-(1-fluoro-2-(2-methyloxazol-4-yl)ethenyl)-8,8,12,16-tetramethyl-4,13,17-trioxabicyclo[14.1.0]heptadecane-5,9-dione
(4S,7R,8S,9R,13(Z),16S(Z))-4,8-Dihydroxy-1,10-dioxa-16-(1-fluoro-2-(2-methyloxazol-4-yl)ethenyl)-7-(prop-2-en-1-yl)-5,5,9,13-tetramethyl-cyclohexadec-13-ene-2,6-dione
(1S,3S(Z),7S,10R,11S,12R,16R)-7,11-Dihydroxy-3-(1-fluoro-2-(2-methyloxazol-4-yl)ethenyl)-10-(prop-2-en-1-yl)-8,8,12,16-tetramethyl-4,13,17-trioxabicyclo[14.1.0]heptadecane-5,9-dione
(4S,7R,8S,9R,13(Z),16S(Z))-4,8-Dihydroxy-1,10-dioxa-16-(1-fluoro-2-(2-pyridyl)ethenyl)-5,5,7,9,13-pentamethyl-cyclohexadec-13-ene-2,6-dione
(1S,3S(Z),7S,10R,11S,12R,16R)-7,11-Dihydroxy-3-(1-fluoro-2-(2-pyridyl)ethenyl)-8,8,10,12,16-pentamethyl-4,13,17-trioxabicyclo[14.1.0]heptadecane-5,9-dione
(4S,7R,8S,9R,13(Z),16S(Z))-4,8-Dihydroxy-1,10-dioxa-7-ethyl-16-(1-fluoro-2-(2-pyridyl)ethenyl)-5,5,9,13-tetramethyl-cyclohexadec-13-ene-2,6-dione
(1S,3S(Z),7S,10R,11S,12R,16R)-7,11-Dihydroxy-10-ethyl-3-(1-fluoro-2-(2-pyridyl)ethenyl)-8,8,12,16-tetramethyl-4,13,17-trioxabicyclo[14.1.0]heptadecane-5,9-dione
(4S,7R,8S,9R,13(Z),16S(Z))-4,8-Dihydroxy-1,10-dioxa-16-(1-fluoro-2-(2-pyridyl)ethenyl)-7-(prop-2-en-1-yl)-5,5,9,13-tetramethyl-cyclohexadec-13-ene-2,6-dione
(1S,3S(Z),7S,10R,11S,12R,16R)-7,11-Dihydroxy-3-(1-fluoro-2-(2-pyridyl)ethenyl)-10-(prop-2-en-1-yl)-8,8,12,16-tetramethyl-4,13,17-trioxabicyclo[14.1.0]heptadecane-5,9-dione
(4S,7R,8S,9R,13(Z),16S(Z))-4,8-Dihydroxy-16-(1-fluoro-2-(2-methyl-4-thiazolyl)ethenyl)-1-aza-10-oxa-5,5,7,9,13-pentamethyl-cyclohexadec-13-ene-2,6-dione
(1S,3S(Z),7S,10R,11S,12R,16R)-7,11-Dihydroxy-3-(1-fluoro-2-(2-methyl-4-thiazolyl)ethenyl)-8,8,10,12,16-pentamethyl-4-aza-13,17-dioxabicyclo[14.1.0]heptadecane-5,9-dione
(4S,7R,8S,9R,13(Z),16S(Z))-4,8-Dihydroxy-7-ethyl-16-(1-fluoro-2-(2-methyl-4-thiazolyl)ethenyl)-1-aza-10-oxa-5,5,9,13-tetramethyl-cyclohexadec-13-ene-2,6-dione
(1S,3S(Z),7S,10R,11S,12R,16R)-7,11-Dihydroxy-10-ethyl-3-(1-fluoro-2-(2-methyl-4-thiazolyl)ethenyl)-8,8,12,16-tetramethyl-4-aza-13,17-dioxabicyclo[14.1.0]heptadecane-5,9-dione
(4S,7R,8S,9R,13(Z),16S(Z))-4,8-Dihydroxy-16-(1-fluoro-2-(2-methyl-4-thiazolyl)ethenyl)-7-(prop-2-en-1-yl)-1-aza-10-oxa-5,5,9, 13-tetramethyl-cyclohexadec-13-ene-2,6-dione
(1S,3S(Z),7S,10R,11S,12R,16R)-7,11-Dihydroxy-3-(1-fluoro-2-(2-methyl-4-thiazolyl)ethenyl)-10-(prop-2-en-1-yl)-8,8,12,16-tetramethyl-4-aza-13,17-dioxabicyclo[14.1.0]-heptadecane-5,9-dione
(4S,7R,8S,9R,13(Z),16S(Z))-4,8-Dihydroxy-16-(1-fluoro-2-(2-methyloxazol-4-yl)ethenyl)-1-aza-10-oxa-5,5,7,9,13-pentamethyl-cyclohexadec-13-ene-2,6-dione
(1S,3S(Z),7S,10R,11S,12R,16R)-7,11-Dihydroxy-3-(1-fluoro-2-(2-methyloxazol-4-yl)ethenyl)-8,8,10,12,16-pentamethyl-4-aza-13,17-dioxabicyclo[14.1.0]heptadecane-5,9-dione
(4S,7R,8S,9R,13(Z),16S(Z))-4,8-Dihydroxy-7-ethyl-16-(1-fluoro-2-(2-methyloxazol-4-yl)ethenyl)-1-aza-10-oxa-5,5,9,13-tetramethyl-cyclohexadec-13-ene-2,6-dione
(1S,3S(Z),7S,10R,11S,12R,16R)-7,11-Dihydroxy-110-ethyl-3-(1-fluoro-2-(2-methyloxazol-4-yl)ethenyl)-8,8,12,16-tetramethyl-4-aza-13,17-dioxabicyclo[14.1.0]heptadecane-5,9-dione
(4S,7R,8S,9R,13(Z),16S(Z))-4,8-Dihydroxy-16-(1-fluoro-2-(2-methyloxazol-4-yl)ethenyl)-7-(prop-2-en-1-yl)-1-aza-10-oxa-5,5,9,13-tetramethyl-cyclohexadec-13-ene-2,6-dione
(1S,3S(Z),7S,10R,11S,12R,16R)-7,11-Dihydroxy-3-(1-fluoro-2-(2-methyloxazol-4-yl)ethenyl)-10-(prop-2-en-1-yl)-8,8,12,16-tetramethyl-4-aza-13,17-dioxabicyclo[14.1.0]-heptadecane-5,9-dione
(4S,7R,8S,9R,13(Z),16S(Z))-4,8-Dihydroxy-16-(1-fluoro-2-(2-pyridyl)ethenyl)-1-aza-10-oxa-5,5,7,9,13-pentamethyl-cyclohexadec-13-ene-2,6-dione
(1S,3S(Z),7S,10R,11S,12R,16R)-7,11-Dihydroxy-3-(1-fluoro-2-(2-pyridyl)ethenyl)-8,8,10,12,16-pentamethyl-4-aza-13,17-dioxabicyclo[14.1.0]heptadecane-5,9-dione
(4S,7R,8S,9R,13(Z),16S(Z))-4,8-Dihydroxy-7-ethyl-16-(1-fluoro-2-(2-pyridyl)ethenyl)-1-aza-10-oxa-5,5,9,13-tetramethyl-cyclohexadec-13-ene-2,6-dione
(1S,3S(Z),7S,10R,11S,12R,16R)-7,11-Dihydroxy-10-ethyl-3-(1-fluoro-2-(2-pyridyl)ethenyl)-8,8,12,16-tetramethyl-4-aza-13,17-dioxabicyclo[14.1.0]heptadecane-5,9-dione
(4S,7R,8S,9R,13(Z),16S(Z))-4,8-Dihydroxy-16-(1-fluoro-2-(2-pyridyl)ethenyl)-7-(prop-2-en-1-yl)-1-aza-10-oxa-5,5,9,13-tetramethyl-cyclohexadec-13-ene-2,6-dione
(1S,3S(Z),7S,10R,11S,12R,16R)-7,11-Dihydroxy-3-(1-fluoro-2-(2-pyridyl)ethenyl)-10-(prop-2-en-1-yl)-8,8,12,16-tetramethyl-4-aza-13,17-dioxabicyclo[14.1.0]heptadecane-5,9-dione
(4S,7R,8S,9R,13(Z),16S(Z))-4,8-Dihydroxy-1,10-dioxa-16-(1-chloro-2-(2-methyl-4-thiazolyl)ethenyl)-5,5,7,9,13-pentamethyl-cyclohexadec-13-ene-2,6-dione
(1S,3S(Z),7S,10R,11S,12R,16R)-7,11-Dihydroxy-3-(1-chloro-2-(2-methyl-4-thiazolyl)ethenyl)-8,8,10,12,16-pentamethyl-4,13,17-trioxabicyclo[14.1.0]heptadecane-5,9-dione
(4S,7R,8S,9R,13(Z),16S(Z))-4,8-Dihydroxy-1,10-dioxa-7-ethyl-16-(1-chloro-2-(2-methyl-4-thiazolyl)ethenyl)-5,5,9,13-tetramethyl-cyclohexadec-13-ene-2,6-dione
(1S,3S(Z),7S,10R,11S,12R,16R)-7,11-Dihydroxy-10-ethyl-3-(1-chloro-2-(2-methyl-4-thiazolyl)ethenyl)-8,8,12,16-tetramethyl-4,13,17-trioxabicyclo[14.1.0]heptadecane-5,9-dione
(4S,7R,8S,9R,13(Z),16S(Z))-4,8-Dihydroxy-1,10-dioxa-16-(1-chloro-2-(2-methyl-4-thiazolyl)ethenyl)-7-(prop-2-en-1-yl)-5,5,9,13-tetramethyl-cyclohexadec-13-ene-2,6-dione
(1S,3S(Z),7S,10R,11S,12R,16R)-7,11-Dihydroxy-3-(1-chloro-2-(2-methyl-4-thiazolyl)ethenyl)-10-(prop-2-en-1-yl)-8,8,12,16-tetramethyl-4,13,17-trioxabicyclo[14.1.0]-heptadecane-5,9-dione
(4S,7R,8S,9R,13(Z),16S(Z))-4,8-Dihydroxy-1,10-dioxa-16-(1-chloro-2-(2-methyloxazol-4-yl)ethenyl)-5,5,7,9,13-pentamethyl-cyclohexadec-13-ene-2,6-dione
(1S,3S(Z),7S,10R,11S,12R,16R)-7,11-Dihydroxy-3-(1-chloro-2-(2-methyloxazol-4-yl)ethenyl)-8,8,10,12,16-pentamethyl-4,13,17-trioxabicyclo[14.1.0]heptadecane-5,9-dione
(4S,7R,8S,9R,13(Z),16S(Z))-4,8-Dihydroxy-1,10-dioxa-7-ethyl-16-(1-chloro-2-(2-methyloxazol-4-yl)ethenyl)-5,5,9,13-tetramethyl-cyclohexadec-13-ene-2,6-dione
(1S,3S(Z),7S,10R,11S,12R,16R)-7,11-Dihydroxy-10-ethyl-3-(1-chloro-2-(2-methyloxazol-4-yl)ethenyl)-8,8,12,16-tetramethyl-4,13,17-trioxabicyclo[14.1.0]heptadecane-5,9-dione
(4S,7R,8S,9R,13(Z),16S(Z))-4,8-Dihydroxy-1,10-dioxa-16-(1-chloro-2-(2-methyloxazol-4-yl)ethenyl)-7-(prop-2-en-1-yl)-5,5,9,13-tetramethyl-cyclohexadec-13-ene-2,6-dione
(1S,3S(Z),7S,10R,11S,12R,16R)-7,11-Dihydroxy-3-(1-chloro-2-(2-methyloxazol-4-yl)ethenyl)-10-(prop-2-en-1-yl)-8,8,12,16-tetramethyl-4,13,17-trioxabicyclo[14.1.0]heptadecane-5,9-dione
(4S,7R,8S,9R,13(Z),16S(Z))-4,8-Dihydroxy-1,10-dioxa-16-(1-chloro-2-(2-pyridyl)ethenyl)-5,5,7,9,13-pentamethyl-cyclohexadec-13-ene-2,6-dione
(1S,3S(Z),7S,10R,11S,12R,16R)-7,11-Dihydroxy-3-(1-chloro-2-(2-pyridyl)ethenyl)-8,8,10,12,16-pentamethyl-4,13,17-trioxabicyclo[14.1.0]heptadecane-5,9-dione
(4S,7R,8S,9R,13(Z),16S(Z))-4,8-Dihydroxy-1,10-dioxa-7-ethyl-16-(1-chloro-2-(2-pyridyl)ethenyl)-5,5,9,13-tetramethyl-cyclohexadec-13-ene-2,6-dione
(1S,3S(Z),7S,10R,11S,12R,16R)-7,11-Dihydroxy-10-ethyl-3-(1-chloro-2-(2-pyridyl)ethenyl)-8,8,12,16-tetramethyl-4,13,17-trioxabicyclo[14.1.0]heptadecane-5,9-dione
(4S,7R,8S,9R,13(Z),16S(Z))-4,8-Dihydroxy-1,10-dioxa-16-(1-chloro-2-(2-pyridyl)ethenyl)-7-(prop-2-en-1-yl)-5,5,9,13-tetramethyl-cyclohexadec-13-ene-2,6-dione
(1S,3S(Z),7S,10R,11S,12R,16R)-7,11-Dihydroxy-3-(1-chloro-2-(2-pyridyl)ethenyl)-10-(prop-2-en-1-yl)-8,8,12,16-tetramethyl-4,13,17-trioxabicyclo[14.1.0]heptadecane-5,9-dione
(4S,7R,8S,9R,13(Z),16S(Z))-4,8-Dihydroxy-16-(1-chloro-2-(2-methyl-4-thiazolyl)ethenyl)-1-aza-10-oxa-5,5,7,9,13-pentamethyl-cyclohexadec-13-ene-2,6-dione
(1S,3S(Z),7S,10R,11S,12R,16R)-7,11-Dihydroxy-3-(1-chloro-2-(2-methyl-4-thiazolyl)ethenyl)-8,8,10,12,16-pentamethyl-4-aza-13,17-dioxabicyclo[14.1.0]heptadecane-5,9-dione
(4S,7R,8S,9R,13(Z),16S(Z))-4,8-Dihydroxy-7-ethyl-16-(1-chloro-2-(2-methyl-4-thiazolyl)ethenyl)-1-aza-10-oxa-5,5,9,13-tetramethyl-cyclohexadec-13-ene-2,6-dione
(1S,3S(Z),7S,10R,11S,12R,16R)-7,11-Dihydroxy-10-ethyl-3-(1-chloro-2-(2-methyl-4-thiazolyl)ethenyl)-8,8,12,16-tetramethyl-4-aza-13,17-dioxabicyclo[14.1.0]heptadecane-5,9-dione
(4S,7R,8S,9R,13(Z),16S(Z))-4,8-Dihydroxy-16-(1-chloro-2-(2-methyl-4-thiazolyl)ethenyl)-7-(prop-2-en-1-yl)-1-aza-10-oxa-5,5,9,13-tetramethyl-cyclohexadec-13-ene-2,6-dione
(1S,3S(Z),7S,10R,11S,12R,16R)-7,11-Dihydroxy-3-(1-chloro-2-(2-methyl-4-thiazolyl)ethenyl)-10-(prop-2-en-1-yl)-8,8,12,16-tetramethyl-4-aza-13,17-dioxabicyclo[14.1.0]-heptadecane-5,9-dione
(4S,7R,8S,9R,13(Z),16S(Z))-4,8-Dihydroxy-16-(1-chloro-2-(2-methyloxazol-4-yl)ethenyl)-1-aza-10-oxa-5,5,7,9,13-pentamethyl-cyclohexadec-13-ene-2,6-dione
(1S,3S(Z),7S,10R,11S,12R,16R)-7,11-Dihydroxy-3-(1-chloro-2-(2-methyloxazol-4-yl)ethenyl)-8,8,10,12,16-pentamethyl-4-aza-13,17-dioxabicyclo[14.1.0]heptadecane-5,9-dione
(4S,7R,8S,9R,13(Z),16S(Z))-4,8-Dihydroxy-7-ethyl-16-(1-chloro-2-(2-methyloxazol-4-yl)ethenyl)-1-aza-10-oxa-5,5,9,13-tetramethyl-cyclohexadec-13-ene-2,6-dione
(1S,3S(Z),7S,10R,11S, 12R,16R)-7,11-Dihydroxy-10-ethyl-3-(1-chloro-2-(2-methyloxazol-4-yl)ethenyl)-8,8,12,16-tetramethyl-4-aza-13,17-dioxabicyclo[14.1.0]heptadecane-5,9-dione
(4S,7R,8S,9R,13(Z),16S(Z))-4,8-Dihydroxy-16-(1-chloro-2-(2-methyloxazol-4-yl)ethenyl)-7-(prop-2-en-1-yl)-1-aza-10-oxa-5,5,9,13-tetramethyl-cyclohexadec-13-ene-2,6-dione
(1S,3S(Z),7S,10R,11S,12R,16R)-7,11-Dihydroxy-3-(1-chloro-2-(2-methyloxazol-4-yl)ethenyl)-10-(prop-2-en-1-yl)-8,8,12,16-tetramethyl-4-aza-13,17-dioxabicyclo[14.1.0]-heptadecane-5,9-dione
(4S,7R,8S,9R,13(Z),16S(Z))-4,8-Dihydroxy-16-(1-chloro-2-(2-pyridyl)ethenyl)-1-aza-10-oxa-5,5,7,9,13-pentamethyl-cyclohexadec-13-ene-2,6-dione
(1S,3S(Z),7S,10R,11S,12R,16R)-7,11-Dihydroxy-3-(1-chloro-2-(2-pyridyl)ethenyl)-8,8,10,12,16-pentamethyl-4-aza-13,17-dioxabicyclo[14.1.0]heptadecane-5,9-dione
(4S,7R,8 S,9R,13(Z),16S(Z))-4,8-Dihydroxy-7-ethyl-16-(1-chloro-2-(2-pyridyl)ethenyl)-1-aza-10-oxa-5,5,9,13-tetramethyl-cyclohexadec-13-ene-2,6-dione
(1S,3S(Z),7S,10R,11S,12R,16R)-7,11-Dihydroxy-10-ethyl-3-(1-chloro-2-(2-pyridyl)ethenyl)-8,8,12,16-tetramethyl-4-aza-13,17-dioxabicyclo[14.1.0]heptadecane-5,9-dione
(4S,7R,8S,9R,13(Z),16S(Z))-4,8-Dihydroxy-16-(1-chloro-2-(2-pyridyl)ethenyl)-7-(prop-2-en-1-yl)-1-aza-10-oxa-5,5,9, 13-tetramethyl-cyclohexadec-13-ene-2,6-dione
(1S,3S(Z),7S,10R,11S,12R,16R)-7,11-Dihydroxy-3-(1-chloro-2-(2-pyridyl)ethenyl)-10-(prop-2-en-1-yl)-8,8,12,16-tetramethyl-4-aza-13,17-dioxabicyclo[14.1.0]heptadecane-5,9-dione
(4S,7R,8S,9R,13(Z),16S (Z))-4,8-Dihydroxy-1,10-dioxa-16-(1-fluoro-2-(2-methyl-4-thiazolyl)ethenyl)-7,9,13-trimethyl-5,5-(1,3-trimethylene)cyclohexadec-13-ene-2,6-dione
(1S,3S(Z),7S,10R,11S,12R,16R)-7,11-Dihydroxy-3-(1-fluoro-2-(2-methyl-4-thiazolyl)ethenyl)-10,12,16-trimethyl-8,8-(1,3-trimethylene)-4,13,17-trioxabicyclo[14.1.0]hepta-deca-5,9-dione
(4S,7R,8S,9R,13(Z),16S(Z))-4,8-Dihydroxy-1,10-dioxa-9,13-dimethyl-7-ethyl-16-(1-fluoro-2-(2-methyl-4-thiazolyl)ethenyl)-5,5-(1,3-trimethylene)cyclohexadec-13-ene-2,6-dione
(1S,3S(Z),7S,10R,11S,12R,16R)-7,11-Dihydroxy-12,16-dimethyl-10-ethyl-3-(1-fluoro-2-(2-methyl-4-thiazolyl)ethenyl)-8,8-(1,3-trimethylene)-4,13,17-trioxabicyclo[14.1.0]hepta-decane-5,9-dione
(4S,7R,8S,9R,13(Z),16S(Z))-4,8-Dihydroxy-1,10-dioxa-9,13-dimethyl-16-(1-fluoro-2-(2-methyl-4-thiazolyl)ethenyl)-7-(prop-2-en-1-yl)-5,5-(1,3-trimethylene)cyclohexadec-13-ene-2,6-dione
(1S,3S(Z),7S,10R,11S,12R,16R)-7,11-Dihydroxy-12,16-dimethyl-3-(1-fluoro-2-(2-methyl-4-thiazolyl)ethenyl)-10-(prop-2-en-1-yl)-8,8-(1,3-trimethylene)-4,13,17-trioxabicyclo[14.1.0]hepta-decane-5,9-dione
4S,7R,8S,9R,13(Z),16S(Z))-4,8-Dihydroxy-16-(1-fluoro-2-(2-methyl-4-thiazolyl)ethenyl)-1-aza-10-oxa-7,9,13-trimethyl-5,5-(1,3-trimethylene)cyclohexadec-13-ene-2,6-dione
(1S,3S(Z),7S,10R,11S,12R,16R)-7,11-Dihydroxy-3-(1-fluoro-2-(2-methyl-4-thiazolyl)ethenyl)-10,12,16-trimethyl-8,8-(1,3-trimethylene)-4-aza-13,17-dioxabicyclo-[14.1.0]hepta-deca-5,9-dione
(4S,7R,8S,9R,13(Z),16S(Z))-4,8-Dihydroxy-9,13-dimethyl-7-ethyl-16-(1-fluoro-2-(2-methyl-4-thiazolyl)ethenyl)-1-aza-10-oxa-5,5-(1,3-trimethylene)cyclohexadec-13-ene-2,6-dione
(1S,3S(Z),7S,10R,11S,12R,16R)-7,11-Dihydroxy-12,16-dimethyl-10-ethyl-3-(1-fluoro-2-(2-methyl-4-thiazolyl)ethenyl)-8,8-(1,3-trimethylene)-4-aza-13,17-dioxabicyclo[14.1.0]hepta-decane-5,9-dione
(4S,7R,8S,9R,13(Z),16S(Z))-4,8-Dihydroxy-9,13-dimethyl-16-(1-fluoro-2-(2-methyl-4-thiazolyl)ethenyl)-7-(prop-2-en-1-yl)-1-aza-10-oxa-5,5-(1,3-trimethylene)cyclohexadec-13-ene-2,6-dione
(1S,3S(Z),7S,10R,11S,12R,16R)-7,11-Dihydroxy-12,16-dimethyl-3-(1-fluoro-2-(2-methyl-4-thiazolyl)ethenyl)-10-(prop-2-en-1-yl)-8,8-(1,3-trimethylene)-4-aza-13,17-dioxabicyclo[14.1.0]hepta-decane-5,9-dione
(4S,7R,8S,9R,13(Z),16S(Z))-4,8-Dihydroxy-1,10-dioxa-16-(1-chloro-2-(2-methyl-4-thiazolyl)ethenyl)-7,9,13-trimethyl-5,5-(1,3-trimethylene)cyclohexadec-13-ene-2,6-dione
(1S,3S(Z),7S,10R,11S,12R,16R)-7,11-Dihydroxy-3-(1-chloro-2-(2-methyl-4-thiazolyl)ethenyl)-10,12,16-trimethyl-8,8-(1,3-trimethylene)-4,13,17-trioxabicyclo[14.1.0]hepta-deca-5,9-dione
(4S,7R,8S,9R,13(Z),16S(Z))-4,8-Dihydroxy-1,10-dioxa-9,13-dimethyl-7-ethyl-16-(1-chloro-2-(2-methyl-4-thiazolyl)ethenyl)-5,5-(1,3-trimethylene)cyclohexadec-13-ene-2,6-dione
(1S,3S(Z),7S,10R,11S,12R,16R)-7,11-Dihydroxy-12,16-dimethyl-10-ethyl-3-(1-chloro-2-(2-methyl-4-thiazolyl)ethenyl)-8,8-(1,3-trimethylene)-4,13,17-trioxabicyclo[14.1.0]hepta-decane-5,9-dione
(4S,7R,8S,9R,13(Z),16S(Z))-4,8-Dihydroxy-1,10-dioxa-9,13-dimethyl-16-(1-chloro-2-(2-methyl-4-thiazolyl)ethenyl)-7-(prop-2-en-1-yl)-5,5-(1,3-trimethylene)cyclohexadec-13-ene-2,6-dione
(1S,3S(Z),7S,10R,11S,12R,16R)-7,11-Dihydroxy-12,16-dimethyl-3-(1-chloro-2-(2-methyl-4-thiazolyl)ethenyl)-10-(prop-2-en-1-yl)-8,8-(1,3-trimethylene)-4,13,17-trioxabicyclo[14.1.0]hepta-decane-5,9-dione
4S,7R,8S,9R,13(Z),16S(Z))-4,8-Dihydroxy-16-(1-chloro-2-(2-methyl-4-thiazolyl)ethenyl)-1-aza-10-oxa-7,9,13-trimethyl-5,5-(1,3-trimethylene)cyclohexadec-13-ene-2,6-dione
(1S,3S(Z),7S,10R,11S,12R,16R)-7,11-Dihydroxy-3-(1-chloro-2-(2-methyl-4-thiazolyl)ethenyl)-10,12,16-trimethyl-8,8-(1,3-trimethylene)-4-aza-13,17-dioxabicyclo[14.1.0]-hepta-deca-5,9-dione
(4S,7R,8S,9R,13(Z),16S(Z))-4,8-Dihydroxy-9,13-dimethyl-7-ethyl-16-(1-chloro-2-(2-methyl-4-thiazolyl)ethenyl)-1-aza-10-oxa-5,5-(1,3-trimethylene)cyclohexadec-13-ene-2,6-dione
(1S,3S(Z),7S,10R,11S,12R,16R)-7,11-Dihydroxy-12,16-dimethyl-10-ethyl-3-(1-chloro-2-(2-methyl-4-thiazolyl)ethenyl)-8,8-(1,3-trimethylene)-4-aza-13,17-dioxabicyclo[14.1.0]hepta-decane-5,9-dione
(4S,7R,8S,9R,13(Z),16S(Z))-4,8-Dihydroxy-9,13-dimethyl-16-(1-chloro-2-(2-methyl-4-thiazolyl)ethenyl)-7-(prop-2-en-1-yl)-1-aza-10-oxa-5,5-(1,3-trimethylene)cyclohexadec-13-ene-2,6-dione
(1S,3S(Z),7S,10R,11S,12R,16R)-7,11-Dihydroxy-12,16-dimethyl-3-(1-chloro-2-(2-methyl-4-thiazolyl)ethenyl)-10-(prop-2-en-1-yl)-8,8-(1,3-trimethylene)-4-aza-13,17-dioxabicyclo[14.1.0]hepta-decane-5,9-dione
(4S,7R,8S,9R,13(Z),16S(Z))-4,8-Dihydroxy-1,10-dioxa-16-(1-methyl-2-(2-methyl-4-thiazolyl)ethenyl)-7-(prop-2-en-1-yl)-5,5,9,13-tetramethyl-cyclohexadec-13-ene-2,6-dione
(1S,3S(Z),7S,10R,11S,12R,16R)-7,11-Dihydroxy-3-(1-methyl-2-(2-methyl-4-thiazolyl)ethenyl)-10-(prop-2-en-1-yl)-8,8,12,16-tetramethyl-4,13,17-trioxabicyclo-[14.1.0]heptadecane-5,9-dione
(4S,7R,8S,9R,13(Z),16S(Z))-4,8-Dihydroxy-1,10-dioxa-16-(1-methyl-2-(2-methyloxazol-4-yl)ethenyl)-7-(prop-2-en-1-yl)-5,5,9,13-tetramethyl-cyclohexadec-13-ene-2,6-dione
(1S,3S(Z),7S,10R,11S,12R,16R)-7,11-Dihydroxy-3-(1-methyl-2-(2-methyloxazol-4-yl)ethenyl)-10-(prop-2-en-1-yl)-8,8,12,16-tetramethyl-4,13,17-trioxabicyclo[14.1.0]heptadecane-5,9-dione
(4S,7R,8S,9R,13(Z),16S(Z))-4,8-Dihydroxy-1,10-dioxa-16-(1-methyl-2-(2-pyridyl)ethenyl)-7-(prop-2-en-1-yl)-5,5,9,13-tetramethyl-cyclohexadec-13-ene-2,6-dione
(1S,3S(Z),7S,10R,11S,12R,16R)-7,11-Dihydroxy-3-(1-methyl-2-(2-pyridyl)ethenyl)-10-(prop-2-en-1-yl)-8,8,12,16-tetramethyl-4,13,17-trioxabicyclo[14.1.0]heptadecane-5,9-dione
(4S,7R,8S,9R,13(Z),16S(Z))-4,8-Dihydroxy-16-(1-methyl-2-(2-methyl-4-thiazolyl)ethenyl)-7-(prop-2-en-1-yl)-1-aza-10-oxa-5,5,9,13-tetramethyl-cyclohexadec-13-ene-2,6-dione
(1S,3S(Z),7S,10R,11S,12R,16R)-7,11-Dihydroxy-3-(1-methyl-2-(2-methyl-4-thiazolyl)ethenyl)-10-(prop-2-en-1-yl)-8,8,12,16-tetramethyl-4-aza-13,17-dioxabicyclo[14.1.0]heptadecane-5,9-dione
(4S,7R,8S,9R,13(Z),16S(Z))-4,8-Dihydroxy-16-(1-methyl-2-(2-methyloxazol-4-yl)ethenyl)-7-(prop-2-en-1-yl)-1-aza-10-oxa-5,5,9,13-tetramethyl-cyclohexadec-13-ene-2,6-dione
(1S,3S(Z),7S,10R,11S,12R,16R)-7,11-Dihydroxy-3-(1-methyl-2-(2-methyloxazol-4-yl)ethenyl)-10-(prop-2-en-1-yl)-8,8,12,16-tetramethyl-4-aza-13,17-dioxabicyclo[14.1.0]-heptadecane-5,9-dione
(4S,7R,8S,9R,13(Z),16S(Z))-4,8-Dihydroxy-16-(1-methyl-2-(2-pyridyl)ethenyl)-7-(prop-2-en-1-yl)-1-aza-10-oxa-5,5,9,13-tetramethyl-cyclohexadec-13-ene-2,6-dione
(1S,3S(Z),7S,10R,11S,12R,16R)-7,11-Dihydroxy-3-(1-methyl-2-(2-pyridyl)ethenyl)-1,10-(prop-2-en-1-yl)-8,8,12,16-tetramethyl-4-aza-13,17-dioxabicyclo[14.1.0]heptadecane-5,9-dione
(4S,7R,8S,9R,13(Z),16S(Z))-4,8-Dihydroxy-1,10-dioxa-9,13-dimethyl-16-(1-methyl-2-(2-methyl-4-thiazolyl)ethenyl)-7-(prop-2-en-1-yl)-5,5-(1,3-trimethylene)cyclohexadec-13-ene-2,6-dione
(1S,3S(Z),7S,10R,11S,12R,16R)-7,11-Dihydroxy-12,16-dimethyl-3-(1-methyl-2-(2-methyl-4-thiazolyl)ethenyl)-10-(prop-2-en-1-yl)-8,8-(1,3-trim ethylene)-4,13,17-trioxabicyclo-[14.1.0]hepta-decane-5,9-dione
(4S,7R,8S,9R,13(Z),16S(Z))-4,8-Dihydroxy-9,13-dimethyl-16-(1-methyl-2-(2-methyl-4-thiazolyl)ethenyl)-7-(prop-2-en-1-yl)-1-aza-10-oxa-5,5-(1,3-trimethylene)cyclohexadec-13-ene-2,6-dione
(1S,3S(Z),7S,10R,11S,12R,16R)-7,11-Dihydroxy-12,16-dimethyl-3-(1-methyl-2-(2-methyl-4-thiazolyl)ethenyl)-10-(prop-2-en-1-yl)-8,8-(1,3-trimethylene)-4-aza-13,17-dioxabicyclo[14.1.0]hepta-decane-5,9-dione
(4S,7R,8S,9R,13(Z),16S(Z))-4,8-Dihydroxy-1,10-dioxa-16-(1-methyl-2-(2-methyl-4-thiazolyl)ethenyl)-7-(prop-2-in-1-yl)-5,5,9,13-tetramethyl-cyclohexadec-13-ene-2,6-dione
(1S,3S(Z),7S,10R,11S,12R,16R)-7,11-Dihydroxy-3-(1-methyl-2-(2-methyl-4-thiazolyl)ethenyl)-10-(prop-2-in-1-yl)-8,8,12,16-tetramethyl-4,13,17-trioxabicyclo-[14.1.0]heptadecane-5,9-dione
(4S,7R,8S,9R,13(Z),16S(Z))-4,8-Dihydroxy-1,10-dioxa-16-(1-methyl-2-(2-methyloxazol-4-yl)ethenyl)-7-(prop-2-in-1-yl)-5,5,9,13-tetramethyl-cyclohexadec-13-ene-2,6-dione
(1S,3S(Z),7S,10R,11S,12R,16R)-7,11-Dihydroxy-3-(1-methyl-2-(2-methyloxazol-4-yl)ethenyl)-10-(prop-2-in-1-yl)-8,8,12,16-tetramethyl-4,13,17-trioxabicyclo[14.1.0]heptadecane-5,9-dione
(4S,7R,8S,9R,13(Z),16S(Z))-4,8-Dihydroxy-1,10-dioxa-16-(1-methyl-2-(2-pyridyl)ethenyl)-7-(prop-2-in-1-yl)-5,5,9,13-tetramethyl-cyclohexadec-13-ene-2,6-dione
(1S,3S(Z),7S,10R,11S,12R,16R)-7,11-Dihydroxy-3-(1-methyl-2-(2-pyridyl)ethenyl)-10-(prop-2-in-1-yl)-8,8,12,16-tetramethyl-4,13,17-trioxabicyclo[14.1.0]heptadecane-5,9-dione
(4S,7R,8S,9R,13(Z),16S(Z))-4,8-Dihydroxy-16-(1-methyl-2-(2-methyl-4-thiazolyl)ethenyl)-7-(prop-2-in-1-yl)-1-aza-10-oxa-5,5,9,13-tetramethyl-cyclohexadec-13-ene-2,6-dione
(1S,3S(Z),7S,10R,11S,12R,16R)-7,11-Dihydroxy-3-(1-methyl-2-(2-methyl-4-thiazolyl)ethenyl)-10-(prop-2-in-1-yl)-8,8,12,16-tetramethyl-4-aza-13,17-dioxabicyclo-[14.1.0]heptadecane-5,9-dione
(4S,7R,8S,9R,13(Z),16S(Z))-4,8-Dihydroxy-16-(1-methyl-2-(2-methyloxazol-4-yl)ethenyl)-7-(prop-2-in-1-yl)-1-aza-10-oxa-5,5,9,13-tetramethyl-cyclohexadec-13-ene-2,6-dione
(1S,3S(Z),7S,10R,11S,12R,16R)-7,11-Dihydroxy-3-(1-methyl-2-(2-methyloxazol-4-yl)ethenyl)-10-(prop-2-in-1-yl)-8,8,12,16-tetramethyl-4-aza-13,17-dioxabicyclo[14.1.0]-heptadecane-5,9-dione
(4S,7R,8S,9R,13(Z),16S(Z))-4,8-Dihydroxy-16-(1-methyl-2-(2-pyridyl)ethenyl)-7-(prop-2-in-1-yl)-1-aza-10-oxa-5,5,9,13-tetramethyl-cyclohexadec-13-ene-2,6-dione
(1S,3S(Z),7S,10R,11S,12R,16R)-7,11-Dihydroxy-3-(1-methyl-2-(2-pyridyl)ethenyl)-10-(prop-2-in-1-yl)-8,8,12,16-tetramethyl-4-aza-13,17-dioxabicyclo[14.1.0]heptadecane-5,9-dione
(4S,7R,8S,9R,13(Z),16S(Z))-4,8-Dihydroxy-1,10-dioxa-9,13-dimethyl-16-(1-methyl-2-(2-methyl-4-thiazolyl)ethenyl)-7-(prop-2-in-1-yl)-5,5-(1,3-trimethylene)cyclohexadec-13-ene-2,6-dione
(1S,3S(Z),7S,10R,11S,12R,16R)-7,11-Dihydroxy-12,16-dimethyl-3-(1-methyl-2-(2-methyl-4-thiazolyl)ethenyl)-10-(prop-2-in-1-yl)-8,8-(1,3-trimethylene)-4,13,17-trioxabicyclo[14.1.0]hepta-decane-5,9-dione
(4S,7R,8S,9R,13(Z),16S(Z))-4,8-Dihydroxy-9,13-dimethyl-16-(1-methyl-2-(2-methyl-4-thiazolyl)ethenyl)-7-(prop-2-in-1-yl)-1-aza-10-oxa-5,5-(1,3-trimethylene)cyclohexadec-13-ene-2,6-dione
(1S,3S(Z),7S,10R,11S,12R,16R)-7,11-Dihydroxy-12,16-dimethyl-3-(1-methyl-2-(2-methyl-4-thiazolyl)ethenyl)-10-(prop-2-in-1-yl)-8,8-(1,3-trimethylene)-4-aza-13,17-dioxabicyclo[14.1.0]hepta-decane-5,9-dione
(4S,7R,8S,9R,13(Z),16S(Z))-4,8-Dihydroxy-1,10-dioxa-16-(1-methyl-2-(2-methyl-4-thiazolyl)ethenyl)-7-(but-2-en-1-yl)-5,5,9,13-tetramethyl-cyclohexadec-13-ene-2,6-dione
(1S,3S(Z),7S,10R,11S,12R,16R)-7,11-Dihydroxy-3-(1-methyl-2-(2-methyl-4-thiazolyl)ethenyl)-10-(but-2-en-1-yl)-8,8,12,16-tetramethyl-4,13,17-trioxabicyclo[14.1.0]-heptadecane-5,9-dione
(4S,7R,8S,9R,13(Z),16S(Z))-4,8-Dihydroxy-1,10-dioxa-16-(1-methyl-2-(2-methyloxazol-4-yl)ethenyl)-7-(but-2-en-1-yl)-5,5,9,13-tetramethyl-cyclohexadec-13-ene-2,6-dione
(1S,3S(Z),7S,10R,11S,12R,16R)-7,1-Dihydroxy-3-(1-methyl-2-(2-methyloxazol-4-yl)ethenyl)-10-(but-2-en-1-yl)-8,8,12,16-tetramethyl-4,13,17-trioxabicyclo[14.1.0]heptadecane-5,9-dione
(4S,7R,8S,9R,13(Z),16S(Z))-4,8-Dihydroxy-1,10-dioxa-16-(1-methyl-2-(2-pyridyl)ethenyl)-7-(but-2-en-1-yl)-5,5,9,13-tetramethyl-cyclohexadec-13-ene-2,6-dione
(1S,3S(Z),7S,10R,11S,12R,16R)-7,11-Dihydroxy-3-(1-methyl-2-(2-pyridyl)ethenyl)-10-(but-2-en-1-yl)-8,8,12,16-tetramethyl-4,13,17-trioxabicyclo[14.1.0]heptadecane-5,9-dione
(4S,7R,8S,9R,13(Z),16S(Z))-4,8-Dihydroxy-16-(1-methyl-2-(2-methyl-4-thiazolyl)-ethenyl)-7-(but-2-en-1-yl)-1-aza-10-oxa-5,5,9,13-tetramethyl-cyclohexadec-13-ene-2,6-dione
(1S,3S(Z),7S,10R,11S,12R,16R)-7,11-Dihydroxy-3-(1-methyl-2-(2-methyl-4-thiazolyl)ethenyl)-10-(but-2-en-1-yl)-8,8,12,16-tetramethyl-4-aza-13,17-dioxabicyclo[14.1.0]-heptadecane-5,9-dione
(4S,7R,8S,9R,13(Z),16S(Z))-4,8-Dihydroxy-16-(1-methyl-2-(2-methyloxazol-4-yl)ethenyl)-7-(but-2-en-1-yl)-1-aza-10-oxa-5,5,9,13-tetramethyl-cyclohexadec-13-ene-2,6-dione
(1S,3S(Z),7S,10R,11S,12R,16R)-7,11-Dihydroxy-3-(1-methyl-2-(2-methyloxazol-4-yl)ethenyl)-10-(but-2-en-1-yl)-8,8,12,16-tetramethyl-4-aza-13,17-dioxabicyclo[14.1.0]-heptadecane-5,9-dione
(4S,7R,8S,9R,13(Z),16S(Z))-4,8-Dihydroxy-16-(1-methyl-2-(2-pyridyl)ethenyl)-7-(but-2-en-1-yl)-1-aza-10-oxa-5,5,9,13-tetramethyl-cyclohexadec-13-ene-2,6-dione
(1S,3S(Z),7S,10R,11S,12R,16R)-7,11-Dihydroxy-3-(1-methyl-2-(2-pyridyl)ethenyl)-10-(but-2-en-1-yl)-8,8,12,16-tetramethyl-4-aza-13,17-dioxabicyclo[14.1.0]heptadecane-5,9-dione
(4S,7R,8S,9R,13(Z),16S(Z))-4,8-Dihydroxy-1,10-dioxa-9,13-dimethyl-16-(1-methyl-2-(2-methyl-4-thiazolyl)ethenyl)-7-(but-2-en-1-yl)-5,5-(1,3-trimethylene)cyclohexadec-13-ene-2,6-dione
(1S,3S(Z),7S,10R,11S,12R,16R)-7,11-Dihydroxy-12,16-dimethyl-3-(1-methyl-2-(2-methyl-4-thiazolyl)ethenyl)-10-(but-2-en-1-yl)-8,8-(1,3-trimethylene)-4,13,17-trioxabicyclo[14.1.0]hepta-decane-5,9-dione
(4S,7R,8S,9R,13(Z),16S(Z))-4,8-Dihydroxy-9,13-dimethyl-16-(1-methyl-2-(2-methyl-4-thiazolyl)ethenyl)-7-(but-2-en-1-yl)-1-aza-10-oxa-5,5-(1,3-trimethylene)cyclohexadec-13-ene-2,6-dione
(1S,3S(Z),7S,10R,11S,12R,16R)-7,11-Dihydroxy-12,16-dimethyl-3-(1-methyl-2-(2-methyl-4-thiazolyl)ethenyl)-10-(but-2-en-1-yl)-8,8-(1,3-trimethylene)-4-aza-13,17-dioxabicyclo[14.1.0]hepta-decane-5,9-dione
(4S,7R,8S,9R,13(Z),16S(Z))-4,8-Dihydroxy-1,10-dioxa-16-(1-methyl-2-(2-methyl-4-thiazolyl)ethenyl)-7-(but-2-in-1-yl)-5,5,9, 13-tetramethyl-cyclohexadec-13-ene-2,6-dione
(1S,3S(Z),7S,10R,11S,12R,16R)-7,11-Dihydroxy-3-(1-methyl-2-(2-methyl-4-thiazolyl)ethenyl)-10-(but-2-in-1-yl)-8,8,12,16-tetramethyl-4,13,17-trioxabicyclo[14.1.0]-heptadecane-5,9-dione
(4S,7R,8S,9R,13(Z),16S(Z))-4,8-Dihydroxy-1,10-dioxa-16-(1-methyl-2-(2-methyloxazol-4-yl)ethenyl)-7-(but-2-in-1-yl)-5,5,9,13-tetramethyl-cyclohexadec-13-ene-2,6-dione
(1S,3S(Z),7S,10R,11S,12R,16R)-7,11-Dihydroxy-3-(1-methyl-2-(2-methyloxazol-4-yl)ethenyl)-10-(but-2-in-1-yl)-8,8,12,16-tetramethyl-4,13,17-trioxabicyclo[14.1.0]heptadecane-5,9-dione
(4S,7R,8S,9R,13(Z),16S(Z))-4,8-Dihydroxy-1,10-dioxa-16-(1-methyl-2-(2-pyridyl)ethenyl)-7-(but-2-in-1-yl)-5,5,9,13-tetramethyl-cyclohexadec-13-ene-2,6-dione
(1S,3S(Z),7S,10R,11S,12R,16R)-7,11-Dihydroxy-3-(1-methyl-2-(2-pyridyl)ethenyl)-10-(but-2-in-1-yl)-8,8,12,16-tetramethyl-4,13,17-trioxabicyclo[14.1.0]heptadecane-5,9-dione
(4S,7R,8S,9R,13(Z),16S(Z))-4,8-Dihydroxy-16-(1-methyl-2-(2-methyl-4-thiazolyl)ethenyl)-7-(but-2-in-1-yl)-1-aza-10-oxa-5,5,9,13-tetramethyl-cyclohexadec-13-ene-2,6-dione
(1S,3S(Z),7S,10R,11S,12R,16R)-7,11-Dihydroxy-3-(1-methyl-2-(2-methyl-4-thiazolyl)ethenyl)-10-(but-2-in-1-yl)-8,8,12,16-tetramethyl-4-aza-13,17-dioxabicyclo[14.1.0]-heptadecane-5,9-dione
(4S,7R,8S,9R,13(Z),16S(Z))-4,8-Dihydroxy-16-(1-methyl-2-(2-methyloxazol-4-yl)ethenyl)-7-(but-2-in-1-yl)-1-aza-10-oxa-5,5,9,13-tetramethyl-cyclohexadec-13-ene-2,6-dione
(1S,3S(Z),7S,10R,11S,12R,16R)-7,11-Dihydroxy-3-(1-methyl-2-(2-methyloxazol-4-yl)ethenyl)-10-(but-2-in-1-yl)-8,8,12,16-tetramethyl-4-aza-13,17-dioxabicyclo[14.1.0]-heptadecane-5,9-dione
(4S,7R,8S,9R,13(Z),16S(Z))-4,8-Dihydroxy-16-(1-methyl-2-(2-pyridyl)ethenyl)-7-(but-2-in-1-yl)-1-aza-10-oxa-5,5,9,13-tetramethyl-cyclohexadec-13-ene-2,6-dione
(1S,3S(Z),7S,10R,11S,12R,16R)-7,11-Dihydroxy-3-(1-methyl-2-(2-pyridyl)ethenyl)-10-(but-2-in-1-yl)-8,8,12,16-tetramethyl-4-aza-13,17-dioxabicyclo[14.1.0]heptadecane-5,9-dione
(4S,7R,8S,9R,13(Z),16S(Z))-4,8-Dihydroxy-1,10-dioxa-9,13-dimethyl-16-(1-methyl-2-(2-methyl-4-thiazolyl)ethenyl)-7-(but-2-in-1-yl)-5,5-(1,3-trimethylene)cyclohexadec-13-ene-2,6-dione
(1S,3S(Z),7S,10R,11S,12R,16R)-7,11-Dihydroxy-12,16-dimethyl-3-(1-methyl-2-(2-methyl-4-thiazolyl)ethenyl)-10-(but-2-in-1-yl)-8,8-(1,3-trimethylene)-4,13,17-trioxabicyclo-[14.1.0]hepta-decane-5,9-dione
(4S,7R,8S,9R,13(Z),16S(Z))-4,8-Dihydroxy-9,13-dimethyl-16-(1-methyl-2-(2-methyl-4-thiazolyl)ethenyl)-7-(but-2-in-1-yl)-1-aza-10-oxa-5,5-(1,3-trimethylene)cyclohexadec-13-ene-2,6-dione
(1S,3S(Z),7S,10R,11S,12R,16R)-7,11-Dihydroxy-12,16-dimethyl-3-(1-methyl-2-(2-methyl-4-thiazolyl)ethenyl)-10-(but-2-in-1-yl)-8,8-(1,3-trimethylene)-4-aza-13,17-dioxabicyclo[14.1.0]hepta-decane-5,9-dione
(4S,7R,8S,9R,13(Z),16S(Z))-4,8-Dihydroxy-1,10-dioxa-16-(2-methyl-5-benzoxazolyl)-7-(prop-2-en-1-yl)-5,5,9,13-tetramethyl-cyclohexadec-13-ene-2,6-dione
(1S,3S(Z),7S,10R,11S,12R,16R)-7,11-Dihydroxy-3-(1-methyl-2-(2-methyl-5-benzoxazolyl)-10-(prop-2-en-1-yl)-8,8,12,16-tetramethyl-4,13,17-trioxabicyclo[14.1.0]-heptadecane-5,9-dione
(4S,7R,8S,9R,13(Z),16S(Z))-4,8-Dihydroxy-16-(2-methyl-5-benzoxazolyl)-7-(prop-2-en-1-yl)-1-aza-10-oxa-5,5,9,13-tetramethyl-cyclohexadec-13-ene-2,6-dione
(1S,3S(Z),7S,10R,11S,12R,16R)-7,11-Dihydroxy-3-(2-methyl-5-benzoxazolyl)-10-(prop-2-en-1-yl)-8,8,12,16-tetramethyl-4-aza-13,17-dioxabicyclo[14.1.0]heptadecane-5,9-dione
(4S,7R,8S,9R,13(Z),16S(Z))-4,8-Dihydroxy-1,10-dioxa-9,13-dimethyl-16′-(2-methyl-5-benzoxazolyl)-7-(prop-2-en-1-yl)-5,5-(1,3-trimethylene)cyclohexadec-13-ene-2,6-dione
(1S,3S(Z),7S,10R,11S,12R,16R)-7,11-Dihydroxy-12,16-dimethyl-3-(2-methyl-5-benzoxazolyl)-10-(prop-2-en-1-yl)-8,8-(1,3-trimethylene)-4,13,17-trioxabicyclo[14.1.0]hepta-decane-5,9-dione
(4S,7R,8S,9R,13(Z),16S(Z))-4,8-Dihydroxy-9,13-dimethyl-16-(2-methyl-5-benzoxazolyl)-7-(prop-2-en-1-yl)-1-aza-10-oxa-5,5-(1,3-trimethylene)cyclohexadec-13-ene-2,6-dione
(1S,3S(Z),7S,10R,11S,12R,16R)-7,11-Dihydroxy-12,16-dimethyl-3-(2-methyl-5-benzoxazolyl)-10-(prop-2-en-1-yl)-8,8-(1,3-trimethylene)-4-aza-13,17-dioxabicyclo[14.1.0]hepta-decane-5,9-dione
(4S,7R,8S,9R,13(Z),16S(Z))-4,8-Dihydroxy-1,10-dioxa-16-(2-methyl-5-benzoxazolyl)-7-(prop-2-in-1-yl)-5,5,9,13-tetramethyl-cyclohexadec-13-ene-2,6-dione
(1S,3S(Z),7S,10R,11S,12R,16R)-7,11-Dihydroxy-3-(2-methyl-5-benzoxazolyl)-10-(prop-2-in-1-yl)-8,8,12,16-tetramethyl-4,13,17-trioxabicyclo[14.1.0]heptadecane-5,9-dione
(4S,7R,8S,9R,13(Z),16S(Z))-4,8-Dihydroxy-16-(2-methyl-5-benzoxazolyl)-7-(prop-2-in-1-yl)-1-aza-10-oxa-5,5,9,13-tetramethyl-cyclohexadec-13-ene-2,6-dione
(1S,3S(Z),7S,10R,11S,12R,16R)-7,11-Dihydroxy-3-(2-methyl-5-benzoxazolyl)-10-(prop-2-in-1-yl)-8,8,12,16-tetramethyl-4-aza-13,17-dioxabicyclo[14.1.0]heptadecane-5,9-dione
(4S,7R,8S,9R,13(Z),16S(Z))-4,8-Dihydroxy-1,10-dioxa-9,13-dimethyl-16-(2-methyl-5-benzoxazolyl)-7-(prop-2-in-1-yl)-5,5-(1,3-trimethylene)cyclohexadec-13-ene-2,6-dione
(1S,3S(Z),7S,10R,11S,12R,16R)-7,11-Dihydroxy-12,16-dimethyl-3-(2-methyl-5-benzoxazolyl)-10-(prop-2-in-1-yl)-8,8-(1,3-trimethylene)-4,13,17-trioxabicyclo[14.1.0]hepta-decane-5,9-dione
(4S,7R,8S,9R,13(Z),16S(Z))-4,8-Dihydroxy-9,13-dimethyl-16-(2-methyl-5-benzoxazolyl)-7-(prop-2-in-1-yl)-1-aza-10-oxa-5,5-(1,3-trimethylene)cyclohexadec-13-ene-2,6-dione
(1S,3S(Z),7S,10R,11S,12R,16R)-7,11-Dihydroxy-12,16-dimethyl-3-(2-methyl-5-benzoxazolyl)-10-(prop-2-in-1-yl)-8,8-(1,3-trimethylene)-4-aza-13,17-dioxabicyclo[14.1.0]hepta-decane-5,9-dione
(4S,7R,8S,9R,13(Z),16S(Z))-4,8-Dihydroxy-1,10-dioxa-16-(2-methyl-5-benzoxazolyl)-7-(but-2-en-1-yl)-5,5,9,13-tetramethyl-cyclohexadec-13-ene-2,6-dione
(1S,3S(Z),7S,10R,11S,12R,16R)-7,11-Dihydroxy-3-(2-methyl-5-benzoxazolyl)-10-(but-2-en-1-yl)-8,8,12,16-tetramethyl-4,13,17-trioxabicyclo[14.1.0]heptadecane-5,9-dione
(4S,7R,8 S,9R,13(Z),16S(Z))-4,8-Dihydroxy-16-(2-methyl-5-benzoxazolyl)-7-(but-2-en-1-yl)-1-aza-10-oxa-5,5,9,13-tetramethyl-cyclohexadec-13-ene-2,6-dione
(1S,3S(Z),7S,10R,11S,12R,16R)-7,11-Dihydroxy-3-(2-methyl-5-benzoxazolyl)-10-(but-2-en-1-yl)-8,8,12,16-tetramethyl-4-aza-13,17-dioxabicyclo[14.1.0]heptadecane-5,9-dione
(4S,7R,8S,9R,13(Z),16S(Z))-4,8-Dihydroxy-1,10-dioxa-9,13-dimethyl-16-(2-methyl-5-benzoxazolyl)-7-(but-2-en-1-yl)-5,5-(1,3-trimethylene)cyclohexadec-13-ene-2,6-dione
(1S,3S(Z),7S,10R,11S,12R,16R)-7,11-Dihydroxy-12,16-dimethyl-3-(2-methyl-5-benzoxazolyl)-10-(but-2-en-1-yl)-8,8-(1,3-trimethylene)-4,13,17-trioxabicyclo[14.1.0]hepta-decane-5,9-dione
(4S,7R,8S,9R,13(Z),16S(Z))-4,8-Dihydroxy-9,13-dimethyl-16-(2-methyl-5-benzoxazolyl)-7-(but-2-en-1-yl)-1-aza-10-oxa-5,5-(1,3-trimethylene)cyclohexadec-13-ene-2,6-dione
(1S,3S(Z),7S,10R,11S,12R,16R)-7,11-Dihydroxy-12,16-dimethyl-3-(2-methyl-5-benzoxazolyl)-10-(but-2-en-1-yl)-8,8-(1,3-trimethylene)-4-aza-13,17-dioxabicyclo[14.1.0]hepta-decane-5,9-dione
(4S,7R,8S,9R,13(Z),16S(Z))-4,8-Dihydroxy-1,10-dioxa-16-(2-methyl-5-benzoxazolyl)-7-(but-2-in-1-yl)-5,5,9,13-tetramethyl-cyclohexadec-13-ene-2,6-dione
(1S,3S(Z),7S,10R,11S,12R,16R)-7,11-Dihydroxy-3-(2-methyl-5-benzoxazolyl)-10-(but-2-in-1-yl)-8,8,12,16-tetramethyl-4,13,17-trioxabicyclo[14.1.0]heptadecane-5,9-dione
(4S,7R,8S,9R,13(Z),16S (Z))-4,8-Dihydroxy-16-(2-methyl-5-benzoxazolyl)-7-(but-2-in-1-yl)-1-aza-10-oxa-5,5,9,13-tetramethyl-cyclohexadec-13-ene-2,6-dione
(1S,3S(Z),7S,10R,11S,12R,16R)-7,11-Dihydroxy-3-(2-methyl-5-benzoxazolyl)-10-(but-2-in-1-yl)-8,8,12,16-tetramethyl-4-aza-13,17-dioxabicyclo[14.1.0]heptadecane-5,9-dione
(4S,7R,8S,9R,13(Z),16S(Z))-4,8-Dihydroxy-1,10-dioxa-9,13-dimethyl-16-(2-methyl-5-benzoxazolyl)-7-(but-2-in-1-yl)-5,5-(1,3-trimethylene)cyclohexadec-13-ene-2,6-dione
(1S,3S(Z),7S,10R,11S,12R,16R)-7,11-Dihydroxy-12,16-dimethyl-3-(2-methyl-5-benzoxazolyl)-10-(but-2-in-1-yl)-8,8-(1,3-trimethylene)-4,13,17-trioxabicyclo[14.1.0]hepta-decane-5,9-dione
(4S,7R,8S,9R,13(Z),16S(Z))-4,8-Dihydroxy-9,13-dimethyl-16-(2-methyl-5-benzoxazolyl)-7-(but-2-in-1-yl)-1-aza-10-oxa-5,5-(1,3-trimethylene)cyclohexadec-13-ene-2,6-dione
(1S,3S(Z),7S,10R,11S,12R,16R)-7,11-Dihydroxy-12,16-dimethyl-3-(2-methyl-5-benzoxazolyl)-10-(but-2-in-1-yl)-8,8-(1,3-trimethylene)-4-aza-13,17-dioxabicyclo[14.1.0]hepta-decane-5,9-dione
(4S,7R,8S,9R,13(Z),16S(Z))-4,8-Dihydroxy-1,10-dioxa-16-(2-methyl-5-benzothiazolyl)-7-(prop-2-en-1-yl)-5,5,9,13-tetramethyl-cyclohexadec-13-ene-2,6-dione
(1S,3S(Z),7S,10R,11S,12R,16R)-7,11-Dihydroxy-3-(1-methyl-2-(2-methyl-5-benzothiazolyl)-10-(prop-2-en-1-yl)-8,8,12,16-tetramethyl-4,13,17-trioxabicyclo[14.1.0]-heptadecane-5,9-dione
(4S,7R,8S,9R,13(Z),16S(Z))-4,8-Dihydroxy-16-(2-methyl-5-benzothiazolyl)-7-(prop-2-en-1-yl)-1-aza-10-oxa-5,5,9,13-tetramethyl-cyclohexadec-13-ene-2,6-dione
(1S,3S(Z),7S,10R,11S,12R,16R)-7,11-Dihydroxy-3-(2-methyl-5-benzothiazolyl)-10-(prop-2-en-1-yl)-8,8,12,16-tetramethyl-4-aza-13,17-dioxabicyclo[14.1.0]heptadecane-5,9-dione
(4S,7R,8S,9R,13(Z),16S(Z))-4,8-Dihydroxy-1,10-dioxa-9,13-dimethyl-16-(2-methyl-5-benzothiazolyl)-7-(prop-2-en-1-yl)-5,5-(1,3-trimethylene)cyclohexadec-13-ene-2,6-dione
(1S,3S(Z),7S,10R,11 S,12R,16R)-7,1-Dihydroxy-12,16-dimethyl-3-(2-methyl-5-benzothiazolyl)-10-(prop-2-en-1-yl)-8,8-(1,3-trimethylene)-4,13,17-trioxabicyclo[14.1.0]heptadecane-5,9-dione
(4S,7R,8S,9R,13(Z),16S(Z))-4,8-Dihydroxy-9,13-dimethyl-16-(2-methyl-5-benzothiazolyl)-7-(prop-2-en-1-yl)-1-aza-10-oxa-5,5-(1,3-trimethylene)cyclohexadec-13-ene-2,6-dione
(1S,3S(Z),7S,10R,11S,12R,16R)-7,11-Dihydroxy-12,16-dimethyl-3-(2-methyl-5-benzothiazolyl)-10-(prop-2-en-1-yl)-8,8-(1,3-trimethylene)-4-aza-13,17-dioxabicyclo-[14.1.0]hepta-decane-5,9-dione
(4S,7R,8 S,9R,13(Z),16S(Z))-4,8-Dihydroxy-1,10-dioxa-16-(2-methyl-5-benzothiazolyl)-7-(prop-2-in-1-yl)-5,5,9,13-tetramethyl-cyclohexadec-13-ene-2,6-dione
(1S,3S(Z),7S,10R,11S,12R,16R)-7,11-Dihydroxy-3-(2-methyl-5-benzothiazolyl)-10-(prop-2-in-1-yl)-8,8,12,16-tetramethyl-4,13,17-trioxabicyclo[14.1.0]heptadecane-5,9-dione
(4S,7R,8S,9R,13(Z),16S(Z))-4,8-Dihydroxy-16-(2-methyl-5-benzothiazolyl)-7-(prop-2-in-1-yl)-1-aza-10-oxa-5,5,9,13-tetramethyl-cyclohexadec-13-ene-2,6-dione
(1S,3S(Z),7S,10R,11S,12R,16R)-7,11-Dihydroxy-3-(2-methyl-5-benzothiazolyl)-10-(prop-2-in-1-yl)-8,8,12,16-tetramethyl-4-aza-13,17-dioxabicyclo[14.1.0]heptadecane-5,9-dione
(4S,7R,8S,9R,13(Z),16S(Z))-4,8-Dihydroxy-1,10-dioxa-9,13-dimethyl-16-(2-methyl-5-benzothiazolyl)-7-(prop-2-in-1-yl)-5,5-(1,3-trimethylene)cyclohexadec-13-ene-2,6-dione
(1S,3S(Z),7S,10R,11S,12R,16R)-7,11-Dihydroxy-12,16-dimethyl-3-(2-methyl-5-benzothiazolyl)-10-prop-2-in-1-yl)-8,8-(1,3-trimethylene)-4,13,17-trioxabicyclo[14.1.0]heptadecane-5,9-dione
(4S,7R,8S,9R,13(Z),16S(Z))-4,8-Dihydroxy-9,13-dimethyl-16-(2-methyl-5-benzothiazolyl)-7-(prop-2-in-1-yl)-1-aza-10-oxa-5,5-(1,3-trimethylene)cyclohexadec-13-ene-2,6-dione
(1S,3S(Z),7S,10R,11S,12R,16R)-7,11-Dihydroxy-12,16-dimethyl-3-(2-methyl-5-benzothiazolyl)-10-(prop-2-in-1-yl)-8,8-(1,3-trinethylene)-4-aza-13,17-dioxabicyclo[14.1.0]hepta-decane-5,9-dione
(4S,7R,8S,9R,13(Z),16S(Z))-4,8-Dihydroxy-1,10-dioxa-16-(2-methyl-5-benzothiazolyl)-7-(but-2-en-1-yl)-5,5,9,13-tetramethyl-cyclohexadec-13-ene-2,6-dione
(1S,3S(Z),7S,10R,11S,12R,16R)-7,11-Dihydroxy-3-(2-methyl-5-benzothiazolyl)-10-(but-2-en-1-yl)-8,8,12,16-tetramethyl-4,13,17-trioxabicyclo[14.1.0]heptadecane-5,9-dione
(4S,7R,8S,9R,13(Z),16S(Z))-4,8-Dihydroxy-16-(2-methyl-5-benzothiazolyl)-7-(but-2-en-1-yl)-1-aza-10-oxa-5,5,9,13-tetramethyl-cyclohexadec-13-ene-2,6-dione
(1S,3S(Z),7S,10R,11S,12R,16R)-7,11-Dihydroxy-3-(2-methyl-5-benzothiazolyl)-10-(but-2-en-1-yl)-8,8,12,16-tetramethyl-4-aza-13,17-dioxabicyclo[14.1.0]heptadecane-5,9-dione
(4S,7R,8S,9R,13(Z),16S(Z))-4,8-Dihydroxy-1,10-dioxa-9,13-dimethyl-16-(2-methyl-5-benzothiazolyl)-7-(but-2-en-1-yl)-5,5-(1,3-trimethylene)cyclohexadec-13-ene-2,6-dione
(1S,3S(Z),7S,10R,11S,12R,16R)-7,11-Dihydroxy-12,16-dimethyl-3-(2-methyl-5-benzothiazolyl)-10-(but-2-en-1-yl)-8,8-(1,3-trimethylene)-4,13,17-trioxabicyclo[14.1.0]hepta-decane-5,9-dione
(4S,7R,8S,9R,13(Z),16S(Z))-4,8-Dihydroxy-9,13-dimethyl-16-(2-methyl-5-benzothiazolyl)-7-(but-2-en-1-yl)-1-aza-10-oxa-5,5-(1,3-trimethylene)cyclohexadec-13-ene-2,6-dione
(1S,3S(Z),7S,10R,11S,12R,16R)-7,11-Dihydroxy-12,16-dimethyl-3-(2-methyl-5-benzothiazolyl)-10-(but-2-en-1-yl)-8,8-(1,3-trimethylene)-4-aza-13,17-dioxabicyclo[14.1.0]hepta-decane-5,9-dione
(4S,7R,8S,9R,13(Z),16S(Z))-4,8-Dihydroxy-1,10-dioxa-16-(2-methyl-5-benzothiazolyl)-7-(but-2-in-1-yl)-5,5,9,13-tetramethyl-cyclohexadec-13-ene-2,6-dione
(1S,3S(Z),7S,10R,11S,12R,16R)-7,11-Dihydroxy-3-(2-methyl-5-benzothiazolyl)-10-(but-2-in-1-yl)-8,8,12,16-tetramethyl-4,13,17-trioxabicyclo[14.1.0]heptadecane-5,9-dione
(4S,7R,8S,9R,13(Z),16S(Z))-4,8-Dihydroxy-16-(2-methyl-5-benzothiazolyl)-7-(but-2-in-1-yl)-1-aza-10-oxa-5,5,9,13-tetramethyl-cyclohexadec-13-ene-2,6-dione
(1S,3S(Z),7S,10R,11S,12R,16R)-7,11-Dihydroxy-3-(2-methyl-5-benzothiazolyl)-10-(but-2-in-1-yl)-8,8,12,16-tetramethyl-4-aza-13,17-dioxabicyclo[14.1.0]heptadecane-5,9-dione
(4S,7R,8S,9R,13(Z),16S(Z))-4,8-Dihydroxy-1,10-dioxa-9,13-dimethyl-16-(2-methyl-5-benzothiazolyl)-7-(but-2-in-1-yl)-5,5-(1,3-trimethylene)cyclohexadec-13-ene-2,6-dione
(1S,3S(Z),7S,10R,11S,12R,16R)-7,11-Dihydroxy-12,16-dimethyl-3-(2-methyl-5-benzothiazolyl)-10-(but-2-in-1-yl)-8,8-(1,3-trimethylene)-4,13,17-trioxabicyclo[14.1.0]hepta-decane-5,9-dione
(4S,7R,8S,9R,13(Z),16S(Z))-4,8-Dihydroxy-9,13-dimethyl-16-(2-methyl-5-benzothiazolyl)-7-(but-2-in-1-yl)-1-aza-10-oxa-5,5-(1,3-trimethylene)cyclohexadec-13-ene-2,6-dione
(1S,3S(Z),7S,10R,11S,12R,16R)-7,11-Dihydroxy-12,16-dimethyl-3-(2-methyl-5-benzothiazolyl)-10-(but-2-in-1-yl)-8,8-(1,3-trimethylene)-4-aza-13,17-dioxabicyclo[14.1.0]hepta-decane-5,9-dione
(4S,7R,8S,9S,13E,16S(E))-4,8-Dihydroxy-13-fluoro-16-(1-methyl-2-(2-methyl-4-thiazolyl)ethenyl)-1,10-dioxa-5,5,7,9-tetramethyl-cyclohexadec-13-ene-2,6-dione
(4S,7R,8S,9S,13Z, 16S(E))-4,8-Dihydroxy-13-fluoro-16-(1-methyl-2-(2-methyl-4-thiazolyl)ethenyl)-1,10-dioxa-5,5,7,9-tetramethyl-cyclohexadec-13-ene-2,6-dione
(1S,3S(E),7S,10R,11S,12S,16S)-16-Fluoro-7,11-dihydroxy-3-(1-methyl-2-(2-methyl-4-thiazolyl)ethenyl)-8,8,10,12-tetramethyl-4,13,17-trioxabicyclo[14.1.0]heptadecane-5,9-dione
(1R,3S(E),7S,10R,11S,12S,16R)-16-Fluoro-7,11-dihydroxy-3-(1-methyl-2-(2-methyl-4-thiazolyl)ethenyl)-8,8,10,12-tetramethyl-4,13,17-trioxabicyclo[14.1.0]heptadecane-5,9-dione
(4S,7R,8S,9S,13E,16S(E))-4,8-Dihydroxy-13-chloro-16-(1-methyl-2-(2-methyl-4-thiazolyl)ethenyl)-1,10-dioxa-5,5,7,9-tetraamethyl-cyclohexadec-13-ene-2,6-dione
(4S,7R,8S,9S,13Z, 16S(E))-4,8-Dihydroxy-13-chloro-16-(1-methyl-2-(2-methyl-4-thiazolyl)ethenyl)-1,10-dioxa-5,5,7,9-tetramethyl-cyclohexadec-13-ene-2,6-dione
(1S,3S(E),7S,10R,11S,12S,16S)-16-Chloro-7,11-dihydroxy-3-(1-methyl-2-(2-methyl-4-thiazolyl)ethenyl)-8,8,10,12-tetramethyl-4,13,17-trioxabicyclo[14.1.0]heptadecane-5,9-dione
(1R,3S(E),7S,10R,11S,12S,16R)-16-Chloro-7,11-dihydroxy-3-(1-methyl-2-(2-methyl-4-thiazolyl)ethenyl)-8,8,10,12-tetramethyl-4,13,17-trioxabicyclo[14.1.0]heptadecane-5,9-dione
(4S,7R,8S,9S,13E,16S(E))-4,8-Dihydroxy-7-ethyl-13-fluoro-16-(1-methyl-2-(2-methyl-4-thiazolyl)ethenyl)-1,10-dioxa-5,5,9-trimethyl-cyclohexadec-13-ene-2,6-dione
(1S,3S(E),7S,10R,11S,12S,16S)-16-Fluoro-10-ethyl-7,11-dihydroxy-3-(1-methyl-2-(2-methyl-4-thiazolyl)ethenyl)-8,8,12-trimethyl-4,13,17-trioxabicyclo[14.1.0]heptadecane-5,9-dione
(1R,3S(E),7S,10R,11S,12S,16R)-16-Fluoro-10-ethyl-7,11-dihydroxy-3-(1-methyl-2-(2-methyl-4-thiazolyl)ethenyl)-8,8,12-trimethyl-4,13,17-trioxabicyclo[14.1.0]heptadecane-5,9-dione
(4S,7R,8S,9S,13E,16S(E))-4,8-Dihydroxy-7-allyl-13-chloro-16-(1-methyl-2-(2-methyl-4-thiazolyl)ethenyl)-1,10-dioxa-5,5,9-trimethyl-cyclohexadec-13-ene-2,6-dione
(1S,3S(E),7S,10R,11S,12S,16S)-16-Chloro-10-allyl-7,11-dihydroxy-3-(1-methyl-2-(2-methyl-4-thiazolyl)ethenyl)-8,8,12-trimethyl-4,13,17-trioxabicyclo[14.1.0]heptadecane-5,9-dione
(1R,3S(E),7S,10R,11S,12S,16R)-16-Chloro-10-allyl-7,11-dihydroxy-3-(1-methyl-2-(2-methyl-4-thiazolyl)ethenyl)-8,8,12-trimethyl-4,13,17-trioxabicyclo[14.1.0]heptadecane-5,9-dione
(4S,7R,8S,9S,13E,16S(E))-4,8-Dihydroxy-13-chloro-16-(1-methyl-2-(2-methyl-2-pyridyl)ethenyl)-1,10-dioxa-5,5,7,9-tetraamethyl-cyclohexadec-13-ene-2,6-dione
(1S,3S(E),7S,10R,11S,12S,16S)-16-Chloro-7,11-dihydroxy-3-(1-methyl-2-(2-methyl-2-pyridyl)ethenyl)-8,8,10,12-tetramethyl-4,13,17-trioxabicyclo[14.1.0]heptadecane-5,9-dione
(1R,3S(E),7S,10R,11S,12S,16R)-16-Chloro-7,11-dihydroxy-3-(1-methyl-2-(2-methyl-2-pyridyl)ethenyl)-8,8,10,12-tetramethyl-4,13,17-trioxabicyclo[14.1.0]heptadecane-5,9-dione
(4S,7R,8S,9S,13E,16S(Z))-4,8-Dihydroxy-13-chloro-16-(1-fluoro-2-(2-methyl-4-thiazolyl)ethenyl)-1,10-dioxa-5,5,7,9-tetraamethyl-cyclohexadec-13-ene-2,6-dione
(1S,3S(Z),7S,10R,11S,12S,16S)-16-Chloro-7,11-dihydroxy-3-(1-fluoro-2-(2-methyl-4-thiazolyl)ethenyl)-8,8,10,12-tetramethyl-4,13,17-trioxabicyclo[14.1.0]heptadecane-5,9-dione
(1R,3S(Z),7S,10R,11S,12S,16R)-16-Chloro-7,11-dihydroxy-3-(1-fluoro-2-(2-methyl-4-thiazolyl)ethenyl)-8,8,10,12-tetramethyl-4,13,17-trioxabicyclo[14.1.0]heptadecane-5,9-dione
(4S,7R,8S,9S,13E,16S(Z))-4,8-Dihydroxy-13-chloro-16-(1-chloro-2-(2-methyl-4-thiazolyl)ethenyl)-1,10-dioxa-5,5,7,9-tetraamethyl-cyclohexadec-13-ene-2,6-dione
(1S,3S(Z),7S,10R,11S,12S,16S)-16-Chloro-7,11-dihydroxy-3-(1-chloro-2-(2-methyl-4-thiazolyl)ethenyl)-8,8,10,12-tetramethyl-4,13,17-trioxabicyclo[14.1.0]heptadecane-5,9-dione
(1R,3S(Z),7S,10R,11S,12S,16R)-16-Chloro-7,11-dihydroxy-3-(1-chloro-2-(2-methyl-4-thiazolyl)ethenyl)-8,8,10,12-tetramethyl-4,13,17-trioxabicyclo[14.1.0]heptadecane-5,9-dione
(4S,7R,8S,9S,13E,16S(Z))-4,8-Dihydroxy-13-chloro-16-(1-fluoro-2-(2-methyl-4-thiazolyl)ethenyl)-5,5-trimethylene-1,10-dioxa-7,9-dimethyl-cyclohexadec-13-ene-2,6-dione
(1S,3S(Z),7S,10R,11S,12S,16S)-16-Chloro-7,11-dihydroxy-3-(1-fluoro-2-(2-methyl-4-thiazolyl)ethenyl)-8,8-trimethylene-10,12-dimethyl-4,13,17-trioxabicyclo[14.1.0]heptadecane-5,9-dione
(1R,3S(Z),7S,10R,11S,12S,16R)-16-Chloro-7,11-dihydroxy-3-(1-fluoro-2-(2-methyl-4-thiazolyl)ethenyl)-8,8-trimethylene-10,12-dimethyl-4,13,17-trioxabicyclo[14.1.0]heptadecane-5,9-dione
(4S,7R,8S,9S,13E,16S(Z))-4,8-Dihydroxy-13-chloro-16-(1-chloro-2-(2-methyl-4-thiazolyl)ethenyl)-5,5-trimethylene-1,10-dioxa-7,9-dimethyl-cyclohexadec-13-ene-2,6-dione
(1S,3S(Z),7S,10R,11S,12S,16S)-16-Chloro-7,11-dihydroxy-3-(1-chloro-2-(2-methyl-4-thiazolyl)ethenyl)-8,8-trimethylene-10,12-dimethyl-4,13,17-trioxabicyclo[14.1.0]heptadecane-5,9-dione
(1R,3S(Z),7S,10R,11S,12S,16R)-16-Chloro-7,11-dihydroxy-3-(1-chloro-2-(2-methyl-4-thiazolyl)ethenyl)-8,8-trimethylene-10,12-dimethyl-4,13,17-trioxabicyclo[14.1.0]heptadecane-5,9-dione
(4S,7R,8S,9S,13E,16S(Z))-4,8-Dihydroxy-13-chloro-16-(1-fluoro-2-(2-methyl-4-oxazolyl)ethenyl)-1,10-dioxa-5,5,7,9-tetraamethyl-cyclohexadec-13-ene-2,6-dione
(1S,3S(Z),7S,10R,11S,12S,16S)-16-Chloro-7,11-dihydroxy-3-(1-fluoro-2-(2-methyl-4-oxazolyl)ethenyl)-8,8,10,12-tetramethyl-4,13,17-trioxabicyclo[14.1.0]heptadecane-5,9-dione
(1R,3S(Z),7S,10R,11S,12S,16R)-16-Chloro-7,11-dihydroxy-3-(1-fluoro-2-(2-methyl-4-oxazolyl)ethenyl)-8,8,10,12-tetramethyl-4,13,17-trioxabicyclo[14.1.0]heptadecane-5,9-dione
(4S,7R,8S,9S,13E,16S(Z))-4,8-Dihydroxy-13-chloro-16-(1-fluoro-2-(2-methyl-2-pyridyl)ethenyl)-1,10-dioxa-5,5,7,9-tetramethyl-cyclohexadec-13-ene-2,6-dione
(1S,3S(Z),7S,10R,11S,12S,16S)-16-Chloro-7,11-dihydroxy-3-(1-fluoro-2-(2-methyl-2-pyridyl)ethenyl)-8,8,10,12-tetramethyl-4,13,17-trioxabicyclo[14.1.0]heptadecane-5,9-dione
(1R,3S(Z),7S,10R,11S,12S,16R)-16-Chloro-7,11-dihydroxy-3-(1-fluoro-2-(2-methyl-2-pyridyl)ethenyl)-8,8,10,12-tetramethyl-4,13,17-trioxabicyclo[14.1.0]heptadecane-5,9-dione
(4S,7R,8S,9S,13E,16S(Z))-4,8-Dihydroxy-7-ethyl-13-chloro-16-(1-chloro-2-(2-methyl-2-pyridyl)ethenyl)-1,10-dioxa-5,5,9-trimethyl-cyclohexadec-13-ene-2,6-dione
(1S,3S(Z),7S,10R,11S,12S,16S)-16-Chloro-7,11-dihydroxy-7-ethyl-3-(1-chloro-2-(2-methyl-2-pyridyl)ethenyl)-8,8,12-trimethyl-4,13,17-trioxabicyclo[14.1.0]heptadecane-5,9-dione
(1R,3S(Z),7S,10R,11S,12S,16R)-16-Chloro-7,11-dihydroxy-7-ethyl-3-(1-chloro-2-(2-methyl-2-pyridyl)ethenyl)-8,8,12-trimethyl-4,13,17-trioxabicyclo[14.1.0]heptadecane-5,9-dione
(4S,7R,8S,9S,13E,16S)-4,8-Dihydroxy-13-fluoro-16-(2-methyl-5-benzothiazolyl)-1,10-dioxa-5,5,7,9-tetramethyl-cyclohexadec-13-ene-2,6-dione
(4S,7R,8S,9S,13Z,16S)-4,8-Dihydroxy-13-fluoro-16-(2-methyl-5-benzothiazolyl)-1,10-dioxa-5,5,7,9-tetramethyl-cyclohexadec-13-ene-2,6-dione
(1S,3S,7S,10R,11S,12S,16S)-16-Fluoro-7,11-dihydroxy-3-(2-methyl-5-benzothiazolyl)-8,8,10,12-tetramethyl-4,13,17-trioxabicyclo[14.1.0]heptadecane-5,9-dione
(1R,3S,7S,10R,11S,12S,16R)-16-Fluoro-7,11-dihydroxy-3-(2-methyl-5-benzothiazolyl)-8,8,10,12-tetramethyl-4,13,17-trioxabicyclo[14.1.0]heptadecane-5,9-dione
(4S,7R,8S,9S,13E,16S)-4,8-Dihydroxy-13-chloro-16-(2-methyl-5-benzothiazolyl)-1,10-dioxa-5,5,7,9-tetraamethyl-cyclohexadec-13-ene-2,6-dione
(4S,7R,8S,9S,13Z,16S)-4,8-Dihydroxy-13-chloro-16-(2-methyl-5-benzothiazolyl)-1,10-dioxa-5,5,7,9-tetramethyl-cyclohexadec-13-ene-2,6-dione
(S,3S,7S,10R,11S,12S,16S)-16-Chloro-7,11-dihydroxy-3-(2-methyl-5-benzothiazolyl)-8,8,10,12-tetramethyl-4,13,17-trioxabicyclo[14.1.0]heptadecane-5,9-dione
(1R,3S,7S,10R,11S,12S,16R)-16-Chloro-7,11-dihydroxy-3-(2-methyl-5-benzothiazolyl)-8,8,10,12-tetramethyl-4,13,17-trioxabicyclo[14.1.0]heptadecane-5,9-dione
(4S,7R,8S,9S,13E,16S)-4,8-Dihydroxy-7-ethyl-13-chloro-16-(2-methyl-5-benzothiazolyl)-1,10-dioxa-5,5,9-trimethyl-cyclohexadec-13-ene-2,6-dione
(1S,3S,7S,10R,11S,12S,16S)-16-Chloro-7,11-dihydroxy-10-ethyl-3-(2-methyl-5-benzothiazolyl)-8,8,12-trimethyl-4,13,17-trioxabicyclo[14.1.0]heptadecane-5,9-dione
(1R,3S,7S,10R,11S,12S,16R)-16-Chloro-7,11-dihydroxy-10-ethyl-3-(2-methyl-5-benzothiazolyl)-8,8,12-tetramethyl-4,13,17-trioxabicyclo[14.1.0]heptadecane-5,9-dione
(4S,7R,8S,9S,13E,16S)-4,8-Dihydroxy-7-allyl-13-chloro-16-(2-methyl-5-benzothiazolyl)-1,10-dioxa-5,5,9-trimethyl-cyclohexadec-13-ene-2,6-dione
(1S,3S,7S,10R,11S,12S,16S)-16-Chloro-7,11-dihydroxy-10-allyl-3-(2-methyl-5-benzothiazolyl)-8,8,12-trimethyl-4,13,17-trioxabicyclo[14.1.0]heptadecane-5,9-dione
(1R,3S,7S,10R,11S,12S,16R)-16-Chloro-7,11-dihydroxy-10-allyl-3-(2-methyl-5-benzothiazolyl)-8,8,12-tetramethyl-4,13,17-trioxabicyclo[14.1.0]heptadecane-5,9-dione
(4S,7R,8S,9S,13E,16S)-4,8-Dihydroxy-13-fluoro-16-(2-methyl-5-benzoxazolyl)-1,10-dioxa-5,5,7,9-tetramethyl-cyclohexadec-13-ene-2,6-dione
(4S,7R,8S,9S,13Z,16S)-4,8-Dihydroxy-13-fluoro-16-(2-methyl-5-benzoxazolyl)-1,10-dioxa-5,5,7,9-tetramethyl-cyclohexadec-13-ene-2,6-dione
(1S,3S,7S,10R,11S,12S,16S)-16-Fluoro-7,11-dihydroxy-3-(2-methyl-5-benzoxazolyl)-8,8,10,12-tetramethyl-4,13,17-trioxabicyclo[14.1.0]heptadecane-5,9-dione
(1R,3S,7S,10R,11S,12S,16R)-16-Fluoro-7,11-dihydroxy-3-(2-methyl-5-benzoxazolyl)-8,8,10,12-tetramethyl-4,13,17-trioxabicyclo[14.1.0]heptadecane-5,9-dione
(4S,7R,8S,9S,13 E,16S)-4,8-Dihydroxy-13-chloro-16-(2-methyl-5-benzoxazolyl)-1,10-dioxa-5,5,7,9-tetraamethyl-cyclohexadec-13-ene-2,6-dione
(4S,7R,8S,9S,13Z, 16S)-4,8-Dihydroxy-13-chloro-16-(2-methyl-5-benzoxazolyl)-1,10-dioxa-5,5,7,9-tetramethyl-cyclohexadec-13-ene-2,6-dione
(1S,3S,7S,10R,11S,12S,16S)-16-Chloro-7,11-dihydroxy-3-(2-methyl-5-benzoxazolyl)-8,8,10,12-tetramethyl-4,13,17-trioxabicyclo[14.1.0]heptadecane-5,9-dione
(1R,3S,7S,10R,11S,12S,16R)-16-Chloro-7,11-dihydroxy-3-(2-methyl-5-benzothiazolyl)-8,8,10,12-tetramethyl-4,13,17-trioxabicyclo[14.1.0]heptadecane-5,9-dione
(4S,7R,8S,9S,13E,16S(E))-4,8-Dihydroxy-13-fluoro-16-(1-methyl-2-(2-methyl-4-thiazolyl)ethenyl)-1-aza-10-oxa-5,5,7,9-tetramethyl-cyclohexadec-13-ene-2,6-dione
(4S,7R,8S,9S,13Z, 16S(E))-4,8-Dihydroxy-13-fluoro-16-(1-methyl-2-(2-methyl-4-thiazolyl)ethenyl)-1-aza-10-oxa-5,5,7,9-tetramethyl-cyclohexadec-13-ene-2,6-dione
(1S,3S(E),7S,10R,11S,12S,16S)-16-Fluoro-7,11-dihydroxy-3-(1-methyl-2-(2-methyl-4-thiazolyl)ethenyl)-8,8,10,12-tetramethyl-4-aza-13,17-dioxabicyclo[14.1.0]heptadecane-5,9-dione
(1R,3S(E),7S,10R,11S,12S,16R)-16-Fluoro-7,11-dihydroxy-3-(1-methyl-2-(2-methyl-4-thiazolyl)ethenyl)-8,8,10,12-tetramethyl-4-aza-13,17-dioxabicyclo[14.1.0]heptadecane-5,9-dione
(4S,7R,8S,9S,13E,16S(E))-4,8-Dihydroxy-13-chloro-16-(1-methyl-2-(2-methyl-4-thiazolyl)ethenyl)-1-aza-10-oxa-5,5,7,9-tetraamethyl-cyclohexadec-13-ene-2,6-dione
(4S,7R,8S,9S,13Z,16S(E))-4,8-Dihydroxy-13-chloro-16-(1-methyl-2-(2-methyl-4-thiazolyl)ethenyl)-1-aza-10-oxa-5,5,7,9-tetramethyl-cyclohexadec-13-ene-2,6-dione
(1S,3S(E),7S,10R,11S,12S,16S)-16-Chloro-7,11-dihydroxy-3-(1-methyl-2-(2-methyl-4-thiazolyl)ethenyl)-8,8,10,12-tetramethyl-4-aza-13,17-dioxabicyclo[14.1.0]heptadecane-5,9-dione
(1R,3S(E),7S,10R,11S,12S,16R)-16-Chloro-7,11-dihydroxy-3-(1-methyl-2-(2-methyl-4-thiazolyl)ethenyl)-8,8,10,12-tetramethyl-4-aza-13,17-dioxabicyclo[14.1.0]heptadecane-5,9-dione
(4S,7R,8S,9S,13E,16S(E))-4,8-Dihydroxy-7-ethyl-13-fluoro-16-(1-methyl-2-(2-methyl-4-thiazolyl)ethenyl)-1-aza-10-oxa-5,5,9-trimethyl-cyclohexadec-13-ene-2,6-dione
(1S,3S(E),7S,10R,11S,12S,16S)-16-Fluoro-10-ethyl-7,11-dihydroxy-3-(1-methyl-2-(2-methyl-4-thiazolyl)ethenyl)-8,8,12-trimethyl-4-aza-13,17-dioxabicyclo[14.1.0]heptadecane-5,9-dione
(1R,3S(E),7S,10R,11S,12S,16R)-16-Fluoro-10-ethyl-7,11-dihydroxy-3-(1-methyl-2-(2-methyl-4-thiazolyl)ethenyl)-8,8,12-trimethyl-4-aza-13,17-dioxabicyclo[14.1.0]heptadecane-5,9-dione
(4S,7R,8S,9S,13E,16S(E))-4,8-Dihydroxy-7-allyl-13-chloro-16-(1-methyl-2-(2-methyl-4-thiazolyl)ethenyl)-1-aza-101-oxa-5,5,9-trimethyl-cyclohexadec-13-ene-2,6-dione
(1S,3S(E),7S,10R,11S,12S,16S)-16-Chloro-10-allyl-7,11-dihydroxy-3-(1-methyl-2-(2-methyl-4-thiazolyl)ethenyl)-8,8,12-trimethyl-4-aza-13,17-dioxabicyclo[14.1.0]heptadecane-5,9-dione
(1R,3S(E),7S,10R,11S,12S,16R)-16-Chloro-10-allyl-7,11-dihydroxy-3-(1-methyl-2-(2-methyl-4-thiazolyl)ethenyl)-8,8,12-trimethyl-4-aza-13,17-dioxabicyclo[14.1.0]heptadecane-5,9-dione
(4S,7R,8S,9S,13E,16S(E))-4,8-Dihydroxy-13-chloro-16-(1-methyl-2-(2-methyl-2-pyridyl)ethenyl)-1-aza-10-oxa-5,5,7,9-tetraamethyl-cyclohexadec-13-ene-2,6-dione
(1S,3S(E),7S,10R,11S,12S,16S)-16-Chloro-7,11-dihydroxy-3-(1-methyl-2-(2-methyl-2-pyridyl)ethenyl)-8,8,10,12-tetramethyl-4-aza-13,17-dioxabicyclo[14.1.0]heptadecane-5,9-dione
(1R,3S(E),7S,10R,11S,12S,16R)-16-Chloro-7,11-dihydroxy-3-(1-methyl-2-(2-methyl-2-pyridyl)ethenyl)-8,8,10,12-tetramethyl-4-aza-13,17-dioxabicyclo[14.1.0]heptadecane-5,9-dione
(4S,7R,8S,9S,13 E,16S(Z))-4,8-Dihydroxy-13-chloro-16-(1-fluoro-2-(2-methyl-4-thiazolyl)ethenyl)-1-aza-10-oxa-5,5,7,9-tetraamethyl-cyclohexadec-13-ene-2,6-dione
(1S,3S(Z),7S,10R,11S,12S,16S)-16-Chloro-7,11-dihydroxy-3-(1-fluoro-2-(2-methyl-4-thiazolyl)ethenyl)-8,8,10,12-tetramethyl-4-aza-13,17-dioxabicyclo[14.1.0]heptadecane-5,9-dione
(1R,3S(Z),7S,10R,11S,12S,16R)-16-Chloro-7,11-dihydroxy-3-(1-fluoro-2-(2-methyl-4-thiazolyl)ethenyl)-8,8,10,12-tetramethyl-4-aza-13,17-dioxabicyclo[14.1.0]heptadecane-5,9-dione
(4S,7R,8S,9S,13E,16S(Z))-4,8-Dihydroxy-13-chloro-16-(1-chloro-2-(2-methyl-4-thiazolyl)ethenyl)-1-aza-10-oxa-5,5,7,9-tetraamethyl-cyclohexadec-13-ene-2,6-dione
(1S,3S(Z),7S,10R,11S,12S,16S)-16-Chloro-7,11-dihydroxy-3-(1-chloro-2-(2-methyl-4-thiazolyl)ethenyl)-8,8,10,12-tetramethyl-4-aza-13,17-dioxabicyclo[14.1.0]heptadecane-5,9-dione
(1R,3S(Z),7S,10R,11S,12S,16R)-16-Chloro-7,11-dihydroxy-3-(1-chloro-2-(2-methyl-4-thiazolyl)ethenyl)-8,8,10,12-tetramethyl-4-aza-13,17-dioxabicyclo[14.1.0]heptadecane-5,9-dione
(4S,7R,8S,9S,13E,16S(Z))-4,8-Dihydroxy-13-chloro-16-(1-fluoro-2-(2-methyl-4-thiazolyl)ethenyl)-5,5-trimethylene-1-aza-10-oxa-7,9-dimethyl-cyclohexadec-13-ene-2,6-dione
(1S,3S(Z),7S,10R,11S,12S,16S)-16-Chloro-7,11-dihydroxy-3-(1-fluoro-2-(2-methyl-4-thiazolyl)ethenyl)-8,8-trimethylene-10,12-dimethyl-4-aza-13,17-dioxabicyclo[14.1.0]heptadecane-5,9-dione
(1R,3S(Z),7S,10R,11S,12S,16R)-16-Chloro-7,11-dihydroxy-3-(1-fluoro-2-(2-methyl-4-thiazolyl)ethenyl)-8,8-trimethylene-10,12-dimethyl-4-aza-13,17-dioxabicyclo[14.1.0]heptadecane-5,9-dione
(4S,7R,8S,9S,13E,16S(Z))-4,8-Dihydroxy-13-chloro-16-(1-chloro-2-(2-methyl-4-thiazolyl)ethenyl)-5,5-trimethylene-1-aza-10-oxa-7,9-dimethyl-cyclohexadec-13-ene-2,6-dione
(1S,3S(Z),7S,10R,11S,12S,16S)-16-Chloro-7,11-dihydroxy-3-(1-chloro-2-(2-methyl-4-thiazolyl)ethenyl)-8,8-trimethylene-10,12-dimethyl-4-aza-13,17-dioxabicyclo[14.1.0]heptadecane-5,9-dione
(1R,3S(Z),7S,10R,11S,12S,16R)-16-Chloro-7,11-dihydroxy-3-(1-chloro-2-(2-methyl-4-thiazolyl)ethenyl)-8,8-trimethylene-10,12-dimethyl-4-aza-13,17-dioxabicyclo[14.1.0]heptadecane-5,9-dione
(4S,7R,8S,9S,13E,16S(Z))-4,8-Dihydroxy-13-chloro-16-(1-fluoro-2-(2-methyl-4-oxazolyl)ethenyl)-1-aza-10-oxa-5,5,7,9-tetraamethyl-cyclohexadec-13-ene-2,6-dione
(1S,3S(Z),7S,10R,11S,12S,16S)-16-Chloro-7,11-dihydroxy-3-(1-fluoro-2-(2-methyl-4-oxazolyl)ethenyl)-8,8,10,12-tetramethyl-4-aza-13,17-dioxabicyclo[14.1.0]heptadecane-5,9-dione
(1R,3S(Z),7S,10R,11S,12S,16R)-16-Chloro-7,11-dihydroxy-3-(1-fluoro-2-(2-methyl-4-oxazolyl)ethenyl)-8,8,10,12-tetramethyl-4-aza-13,17-dioxabicyclo[14.1.0]heptadecane-5,9-dione
(4S,7R,8S,9S,13E,16S(Z))-4,8-Dihydroxy-13-chloro-16-(1-fluoro-2-(2-methyl-2-pyridyl)ethenyl)-1-aza-10-oxa-5,5,7,9-tetraamethyl-cyclohexadec-13-ene-2,6-dione
(1S,3S(Z),7S,10R,11S,12S,16S)-16-Chloro-7,11-dihydroxy-3-(1-fluoro-2-(2-methyl-2-pyridyl)ethenyl)-8,8,10,12-tetramethyl-4-aza-13,17-dioxabicyclo[14.1.0]heptadecane-5,9-dione
(1R,3S(Z),7S,10R,11S,12S,16R)-16-Chloro-7,11-dihydroxy-3-(1-fluoro-2-(2-methyl-2-pyridyl)ethenyl)-8,8,10,12-tetramethyl-4-aza-13,17-dioxabicyclo[14.1.0]heptadecane-5,9-dione
(4S,7R,8S,9S,13E,16S(Z))-4,8-Dihydroxy-7-ethyl-13-chloro-16-(1-chloro-2-(2-methyl-2-pyridyl)ethenyl)-1-aza-10-oxa-5,5,9-trimethyl-cyclohexadec-13-ene-2,6-dione
(1S,3S(Z),7S,10R,11S,12S,16S)-16-Chloro-7,11-dihydroxy-7-ethyl-3-(1-chloro-2-(2-methyl-2-pyridyl)ethenyl)-8,8,12-trimethyl-4-aza-13,17-dioxabicyclo[14.1.0]heptadecane-5,9-dione
(1R,3S(Z),7S,10R,11S,12S,16R)-16-Chloro-7,11-dihydroxy-7-ethyl-3-(1-chloro-2-(2-methyl-2-pyridyl)ethenyl)-8,8,12-trimethyl-4-aza-13,17-dioxabicyclo[14.1.0]heptadecane-5,9-dione
(4S,7R,8S,9S,13E,16S)-4,8-Dihydroxy-13-fluoro-16-(2-methyl-5-benzothiazolyl)-1-aza-10-oxa-5,5,7,9-tetramethyl-cyclohexadec-13-ene-2,6-dione
(4S,7R,8S,9S,13Z, 16S)-4,8-Dihydroxy-13-fluoro-16-(2-methyl-5-benzothiazolyl)-1-aza-10-oxa-5,5,7,9-tetramethyl-cyclohexadec-13-ene-2,6-dione
(1S,3S,7S,10R,11S,12S,16S)-16-Fluoro-7,11-dihydroxy-3-(2-methyl-5-benzothiazolyl)-8,8,10,12-tetramethyl-4-aza-13,17-dioxabicyclo[14.1.0]heptadecane-5,9-dione
(1R,3S,7S,10R,11S,12S,16R)-16-Fluoro-7,11-dihydroxy-3-(2-methyl-5-benzothiazolyl)-8,8,10,12-tetramethyl-4-aza-13,17-dioxabicyclo[14.1.0]heptadecane-5,9-dione
(4S,7R,8S,9S,13E,16S)-4,8-Dihydroxy-13-chloro-16-(2-methyl-5-benzothiazolyl)-1-aza-10-oxa-5,5,7,9-tetraamethyl-cyclohexadec-13-ene-2,6-dione
(4S,7R,8S,9S,13Z,16S)-4,8-Dihydroxy-13-chloro-16-(2-methyl-5-benzothiazolyl)-1-aza-10-oxa-5,5,7,9-tetramethyl-cyclohexadec-13-ene-2,6-dione
(1S,3S,7S,10R,11S,12S,16S)-16-Chloro-7,11-dihydroxy-3-(2-methyl-5-benzothiazolyl)-8,8,10,12-tetramethyl-4-aza-13,17-dioxabicyclo[14.1.0]heptadecane-5,9-dione
(1R,3S,7S,10R,11S,12S,16R)-16-Chloro-7,11-dihydroxy-3-(2-methyl-5-benzothiazolyl)-8,8,10,12-tetramethyl-4-aza-13,17-dioxabicyclo[14.1.0]heptadecane-5,9-dione
(4S,7R,8S,9S,13E,16S)-4,8-Dihydroxy-7-ethyl-13-chloro-16-(2-methyl-5-benzothiazolyl)-1-aza-10-oxa-5,5,9-trimethyl-cyclohexadec-13-ene-2,6-dione
(1S,3S,7S,10R,11S,12S,16S)-16-Chloro-7,11-dihydroxy-10-ethyl-3-(2-methyl-5-benzothiazolyl)-8,8,12-trimethyl-4-aza-13,17-dioxabicyclo[14.1.0]heptadecane-5,9-dione
(1R,3S,7S,10R,11S,12S,16R)-16-Chloro-7,11-dihydroxy-10-ethyl-3-(2-methyl-5-benzothiazolyl)-8,8,12-tetramethyl-4-aza-13,17-dioxabicyclo[14.1.0]heptadecane-5,9-dione
(4S,7R,8S,9S,13E,16S)-4,8-Dihydroxy-7-allyl-13-chloro-16-(2-methyl-5-benzothiazolyl)-1-aza-10-oxa-5,5,9-trimethyl-cyclohexadec-13-ene-2,6-dione
(1S,3S,7S,10R,11S,12S,16S)-16-Chloro-7,11-dihydroxy-10-allyl-3-(2-methyl-5-benzothiazolyl)-8,8,12-trimethyl-4-aza-13,17-dioxabicyclo[14.1.0]heptadecane-5,9-dione
(1R,3S,7S,10R,11S,12S,16R)-16-Chloro-7,11-dihydroxy-10-allyl-3-(2-methyl-5-benzothiazolyl)-8,8,12-tetramethyl-4-aza-13,17-dioxabicyclo[14.1.0]heptadecane-5,9-dione
(4S,7R,8S,9S,13E,16S)-4,8-Dihydroxy-13-fluoro-16-(2-methyl-5-benzoxazolyl)-1-aza-10-oxa-5,5,7,9-tetramethyl-cyclohexadec-13-ene-2,6-dione
(4S,7R,8S,9S,13Z, 16S)-4,8-Dihydroxy-13-fluoro-16-(2-methyl-5-benzoxazolyl)-1-aza-10-oxa-5,5,7,9-tetramethyl-cyclohexadec-13-ene-2,6-dione
(1S,3S,7S,10R,11S,12S,16S)-16-Fluoro-7,11-dihydroxy-3-(2-methyl-5-benzoxazolyl)-8,8,10,12-tetramethyl-4-aza-13,17-dioxabicyclo[14.1.0]heptadecane-5,9-dione
(1R,3S,7S,10R,11S,12S,16R)-16-Fluoro-7,11-dihydroxy-3-(2-methyl-5-benzoxazolyl)-8,8,10,12-tetramethyl-4-aza-13,17-dioxabicyclo[14.1. O]heptadecane-5,9-dione
(4S,7R,8S,9S,13E,16S)-4,8-Dihydroxy-13-chloro-16-(2-methyl-5-benzoxazolyl)-1-aza-10-oxa-5,5,7,9-tetraamethyl-cyclohexadec-13-ene-2,6-dione
(4S,7R,8S,9S,13Z, 16S)-4,8-Dihydroxy-13-chloro-16-(2-methyl-5-benzoxazolyl)-1-aza-10-oxa-5,5,7,9-tetramethyl-cyclohexadec-13-ene-2,6-dione
(1S,3S,7S,10R,11S,12S,16S)-16-Chloro-7,11-dihydroxy-3-(2-methyl-5-benzoxazolyl)-8,8,10,12-tetramethyl-4-aza-13,17-dioxabicyclo[14.1.0]heptadecane-5,9-dione
(1R,3S,7S,10R,11S,12S,16R)-16-Chloro-7,11-dihydroxy-3-(2-methyl-5-benzothiazolyl)-8,8,10,12-tetramethyl-4-aza-13,17-dioxabicyclo[14.1.0]heptadecane-5,9-dione

3. Pharmaceutical preparations that contain at least one epothilone derivative of general formula I according to claim 1 as well as a pharmaceutically compatible vehicle.

4. Use of the compounds of general formula I according to claim 1 for the production of pharmaceutical agents.
